(19) United States
(12) Patent Application Publication
(10) Pub. No.: US 2024/0050451 A1
(43) Pub. Date: Feb. 15, 2024

(54) PRECISION CANNABINOID THERAPY FORMULATIONS AND METHODS OF USE

(71) Applicant: MDM Enterprise Solutions Inc., Menlo Park, CA (US)

(72) Inventors: Eleanor DUFF, Portola Valley, CA (US); Kevin SPELMAN, Ashland, OR (US); Scott EAGLE, Menlo Park, CA (US)

(73) Assignee: MDM Enterprise Solutions Inc., Menlo Park, CA (US)

(21) Appl. No.: 18/246,613

(22) PCT Filed: Sep. 24, 2021
(86) PCT No.: PCT/US2021/052021

§ 371 Date: Mar. 24, 2023

Related U.S. Application Data
(60) Provisional application No. 63/083,655, filed on Sep. 25, 2020.

(51) Int. Cl. A61K 31/00; A61K 36/725; A61K 31/4045; A61K 36/185; A61K 36/67; A61K 36/53; A61K 36/539; A61K 36/488; A61K 36/28; A61K 36/81; A61K 36/69; A61K 36/752; A61K 31/202; A61K 36/899; A61K 36/79; A61K 31/685; A61K 31/385; A61K 36/8964; A61K 36/236; A61K 31/405; A61K 36/258; A61K 31/12; A61P 25/00; A61P 25/22; A61K 9/48

(52) U.S. Cl. CPC A61K 31/658; A61K 36/725; A61K 31/4045; A61K 36/185; A61K 36/67; A61K 36/53; A61K 36/539; A61K 36/488; A61K 36/28; A61K 36/81; A61K 36/69; A61K 36/752; A61K 31/202; A61K 36/899; A61K 36/79; A61K 31/685; A61K 31/385; A61K 36/8964; A61K 36/236; A61K 31/405; A61K 36/258; A61K 31/12; A61P 25/00; A61P 25/22; A61K 9/4825

(57) ABSTRACT

Described herein are cannabinoid therapy formulations for sleep, pain, and anxiety conditions. Also provided are methods for improving sleep quality, relieving pain and/or improving anxiety symptoms.

### CROSS REFERENCE TO RELATED APPLICATIONS

This application claims the benefit of the filing date of U.S. Provisional Patent Application No. 63/083,655, filed Sep. 25, 2020, the contents of which are incorporated herein by reference in their entirety.

### FIELD OF THE DISCLOSURE

The present disclosure provides cannabinoid therapy formulations for sleep, pain, and anxiety conditions. In particular, the present disclosure relates to the compositions comprising at least one negative allosteric modulator of the cannabinoid receptor 1 (CB1) (e.g., cannabidiol (CBD)), at least one partial agonist of CB1 (e.g., cannabinol (CBN)), at least one CB1 antagonist or negative allosteric modulator (e.g., cannabigerol (CBG)), or any combinations thereof, and at least one gamma-aminobutyric acid (GABA)A receptor (GABAAR) activator (e.g., Ziziphus spinosa, Passiflora incarnata, Withania somnifera, and/or melatonin), and/or at least one transient receptor potential cation channel, subfamily A, member 1 (TRPA1) activator (e.g., Piper nigrum).

### BACKGROUND OF THE DISCLOSURE

The compositions of the present disclosure are constructed using various strategies: 1) The endocannabinoid systems is a keystone in this approach and is targeted not only by cannabinoids from Cannabis sativa, but also other medicinal plants (e.g. Piper nigrum, Sceletium tortuosum, Magnolia officinalis, etc.) 2) Using a physician's database of thousands of patients, the formulas were informed by clinical data. 3) Eastern medicine is a time-tested perspective that has provided novel active compounds that have become pharmaceuticals from their medicinal plants (see Rawolfia spp.—see reserpine, Coleus forskholli—see forskolin & Artemisia annua—see artemisinin). Effectiveness of medicinal plants over centuries of use is therefore assumed. 4) These systems have also generated a model of constructing complex phytochemical matrices that include synergist and triggering biochemical pathways that converge on a common therapeutic/pharmacological outcome. 5) Overlapping pharmacological pleiotropy has also been a strategy used by traditional medicine. In this case we define pharmacological pleiotropy as a plant compound having multiple targets and thus, multiple effects on a biochemical pathway or pathways that converge on a common beneficial outcome. 6) Precision medicine, an individualized approach to physiological patterns, is harnessed through the use of algorithms to determine which specific formula will best match the needs of the consumer. 7) Network pharmacology is precisely applied to construct each formula through deliberately targeting multiple, but select, pharmacological targets. By carefully constructing each formula, lower doses are needed for each target, any potential for adverse events is reduced, and desired pharmacological outcomes are amplified. Through a novel combination of all of these approaches the model generated is Precision Network Pharmacology (PNP). The endocannabinoid system (ECS) has become of particular interest over the last decade. The recognition of this system is relatively new and the number of targets included in this physiological network continues to expand beyond the cannabinoid receptor 1 and 2 (CB1 and CB2). Iannotti et al., Prog Lipid Res., 62:107-128 (2016). The ECS also includes the vanilloid receptors, PPARγ receptor, several enzymes, and various endogenous compounds. Kaur R. et al., Curr Clin Pharmacol., 11(2):110-117 (2016).

The constituents from Cannabis, such as cannabinoids, terpenes, and flavonoids, are known to target many different targets in the human system, including the ECS. Russo E B and Marcu J., Adv Pharmacol., 80:67-134 (2017).

There is a need to provide more effective and safer Cannabis products for various medical uses.

### BRIEF SUMMARY OF THE DISCLOSURE

One aspect of the present disclosure is directed to the compositions comprising at least one negative allosteric modulator of the cannabinoid receptor 1 (CB1), at least one partial agonist of CB1, at least one CB1 antagonist or negative allosteric modulator, at least one gamma-aminobutyric acid (GABA)A receptor (GABAAR) activator, at least one transient receptor potential cation channel, subfamily A, member 1 (TRPA1) activator, or any combinations thereof. In certain aspects, at least one negative allosteric modulator of CB1 is CBD, at least one partial agonist of CB1 is CBN, and CBD and CBN are present in a predefined ratio by weight. In some aspects, CBD:CBN ratio is about 2, about 3, about 4, about 5, about 6, about 7, about 8, about 9, about 10, about 11, about 12, about 13, about 14, about 15, about 16, about 17, about 18, about 19, or about 20.

In certain aspects, at least one CB1 antagonist or negative allosteric modulator is CBG, at least one negative allosteric modulator of CB1 is CBD, and CBG and CBD are present in a predefined ratio by weight. In some aspects, CBD:CBG ratio is about 1, about 2, about 3, about 4, about 5, about 6, about 7, about 8, about 9, or about 10.

In certain aspects, the compositions of the present disclosure comprise:
(i) about 5 mg to about 80 mg of cannabidiol (CBD), about 1 mg to about 15 mg of cannabinol (CBN), about 1 mg to about 30 mg of cannabigerol (CBG), or any combinations thereof;
(ii) one or more of the following: about 40 mg to 260 mg of Ziziphus spinosa, about 0.1 mg to 4 mg of melatonin, and about 30 mg to about 200 mg of Passiflora incarnata; and
(iii) three or more of the following: about 30 mg to about 150 mg of eicosapentaenoic acid (EPA)/docosahexaenoic acid (DHA), about 1 mg to about 80 mg of Citrus reticulata (e.g., Citrus reticulata Blanco essential oil), about 50 mg to about 200 mg of Withania somnifera, about 30 mg to about 250 mg of Matricaria recutita, about 30 mg to about 220 mg of Lycopus spp., about 50 mg to about 200 mg of Poria cocos, about 5 mg to about 40 mg of Actaea racemosa, about 10 mg to about 150 mg of Pueraria lobata, about 15 mg to about 70 mg of Anemarrhena asphodeloides, about 30 mg to about 120 mg of phosphatidylserine, about 15 mg to about 70 mg of alpha lipoic acid, about 40 mg to about 150 mg of silymarin, about 100 mg to about 280 mg of Schisandra chinensis, about 5 mg to about 50 mg of Cymbopogon flexuosus, about 10 mg to about 120 mg of Humulus lupulus, about 50 mg to about 200 mg of Grindelia spp., about 30 mg to about 150 mg of Ligusticum wallichii, about 5 mg to about 50 mg of 5-hydroxytryptophan (5-HTP), about 15 mg to about 70 mg of paeonol, about 15 mg to about 150 mg of Panax ginseng, about 20 mg to about 100 mg of Rehmannia glutinosa, about 50 mg to about 200 mg of Polygala tenuifolia, about 20 mg to about 100 mg of Lavendula officinalis, about 50 mg to about 200 mg of Scutellaria lateriflora, about 10 mg to about 150 mg of Pueraria lobata, about 5 mg to about 70 mg of Sceletium tortuosum, about 1 mg to about 15 mg of mixed Tocopherols, about 10 mg to about 200 mg of Magnolia officinalis, about 20 mg to about 100 mg of Piper methysticum, and about 1 mg to about 10 mg of Piper nigrum.

In certain aspects, the compositions of the present disclosure comprise:
(i) about 5 mg to about 80 mg of cannabidiol (CBD), about 1 mg to about 15 mg of cannabinol (CBN), about 1 mg to about 30 mg of cannabigerol (CBG), or any combinations thereof, and about 40 mg to 260 mg of Ziziphus spinosa; and
(ii) four or more of the following: about 0.1 mg to 4 mg of melatonin, about 30 mg to about 200 mg of Passiflora incarnata, about 30 mg to about 150 mg of eicosapentaenoic acid (EPA)/docosahexaenoic acid (DHA), about 1 mg to about 80 mg of Citrus reticulata (e.g., Citrus reticulata Blanco essential oil), about 50 mg to about 200 mg of Withania somnifera, about 30 mg to about 250 mg of Matricaria recutita, about 30 mg to about 220 mg of Lycopus spp., about 50 mg to about 200 mg of Poria cocos, about 5 mg to about 40 mg of Actaea racemosa, about 10 mg to about 150 mg of Pueraria lobata, about 15 mg to about 70 mg of Anemarrhena asphodeloides, about 30 mg to about 120 mg of phosphatidylserine, about 15 mg to about 70 mg of alpha lipoic acid, about 40 mg to about 150 mg of silymarin, about 100 mg to about 280 mg of Schisandra chinensis, about 5 mg to about 50 mg of Cymbopogon flexuosus essential oil, about 10 mg to about 120 mg of Humulus lupulus, about 50 mg to about 200 mg of Grindelia spp., about 30 mg to about 150 mg of Ligusticum wallichii, about 5 mg to about 50 mg of 5-hydroxytryptophan (5-HTP), about 15 mg to about 70 mg of paeonol, about 15 mg to about 150 mg of Panax ginseng, about 20 mg to about 100 mg of Rehmannia glutinosa, about 50 mg to about 200 mg of Polygala tenuifolia, about 20 mg to about 100 mg of Lavendula officinalis, about 50 mg to about 200 mg of Scutellaria lateriflora, about 10 mg to about 150 mg of Pueraria lobata, about 5 mg to about 70 mg of Sceletium tortuosum, about 1 mg to about 15 mg of mixed Tocopherols, about 10 mg to about 200 mg of Magnolia officinalis, about 20 mg to about 100 mg of Piper methysticum, and about 1 mg to about 10 mg of Piper nigrum.

In some aspects, the compositions of the present disclosure comprise:
(i) about 5 mg to about 80 mg of cannabidiol (CBD), about 1 mg to about 15 mg of cannabinol (CBN), about 1 mg to about 30 mg of cannabigerol (CBG), or any combinations thereof, and about 0.1 mg to 4 mg of melatonin; and
(ii) four or more of the following: about 40 mg to 260 mg of Ziziphus spinosa, about 30 mg to about 200 mg of Passiflora incarnata; about 30 mg to about 150 mg of eicosapentaenoic acid (EPA)/docosahexaenoic acid (DHA), about 1 mg to about 80 mg of Citrus reticulata (e.g., Citrus reticulata Blanco essential oil), about 50 mg to about 200 mg of Withania somnifera, about 30 mg to about 250 mg of Matricaria recutita, about 30 mg to about 220 mg of Lycopus spp., about 50 mg to about 200 mg of Poria cocos, about 5 mg to about 40 mg of Actaea racemosa, about 10 mg to about 150 mg of Pueraria lobata, about 15 mg to about 70 mg of Anemarrhena asphodeloides, about 30 mg to about 120 mg of phosphatidylserine, about 15 mg to about 70 mg of alpha lipoic acid, about 40 mg to about 150 mg of silymarin, about 100 mg to about 280 mg of Schisandra chinensis, about 5 mg to about 50 mg of Cymbopogon flexuosus essential oil, about 10 mg to about 120 mg of Humulus lupulus, about 50 mg to about 200 mg of Grindelia spp., about 30 mg to about 150 mg of Ligusticum wallichii, about 5 mg to about 50 mg of 5-hydroxytryptophan (5-HTP), about 15 mg to about 70 mg of paeonol, about 15 mg to about 150 mg of Panax ginseng, about 20 mg to about 100 mg of Rehmannia glutinosa, about 50 mg to about 200 mg of Polygala tenuifolia, about 20 mg to about 100 mg of Lavendula officinalis, about 50 mg to about 200 mg of Scutellaria lateriflora, about 10 mg to about 150 mg of Pueraria lobata, about 5 mg to about 70 mg of Sceletium tortuosum, about 1 mg to about 15 mg of mixed Tocopherols, about 10 mg to about 200 mg of Magnolia officinalis, about 20 mg to about 100 mg of Piper methysticum, and about 1 mg to about 10 mg of Piper nigrum.

In certain aspects, the compositions of the present disclosure comprise:
(i) about 5 mg to about 80 mg of cannabidiol (CBD), about 1 mg to about 15 mg of cannabinol (CBN), about 1 mg to about 30 mg of cannabigerol (CBG), or any combinations thereof, and about 30 mg to about 200 mg of Passiflora incarnata; and
(ii) four or more of the following: about 0.1 mg to 4 mg of melatonin; about 40 mg to 260 mg of Ziziphus spinosa, about 30 mg to about 150 mg of eicosapentaenoic acid (EPA)/docosahexaenoic acid (DHA), about 1 mg to about 80 mg of Citrus reticulata (e.g., Citrus reticulata Blanco essential oil), about 50 mg to about 200 mg of Withania somnifera, about 30 mg to about 250 mg of Matricaria recutita, about 30 mg to about 220 mg of Lycopus spp., about 50 mg to about 200 mg of Poria cocos, about 5 mg to about 40 mg of Actaea racemosa, about 10 mg to about 150 mg of Pueraria lobata, about 15 mg to about 70 mg of Anemarrhena asphodeloides, about 30 mg to about 120 mg of phosphatidylserine, about 15 mg to about 70 mg of alpha lipoic acid, about 40 mg to about 150 mg of silymarin, about 100 mg to about 280 mg of Schisandra chinensis, about 5 mg to about 50 mg of Cymbopogon flexuosus essential oil, about 10 mg to about 120 mg of Humulus lupulus, about 50 mg to about 200 mg of Grindelia spp., about 30 mg to about 150 mg of Ligusticum wallichii, about 5 mg to about 50 mg of 5-hydroxytryptophan (5-HTP), about 15 mg to about 70 mg of paeonol, about 15 mg to about 150 mg of Panax ginseng, about 20 mg to about 100 mg of Rehmannia glutinosa, about 50 mg to about 200 mg of Polygala tenuifolia, about 20 mg to about 100 mg of Lavendula officinalis, about 50 mg to about 200 mg of Scutellaria lateriflora, about 50 mg to about 200 mg of Polygala tenuifolia, about 10 mg to about 150 mg of Pueraria lobata, about 5 mg to about 70 mg of Sceletium tortuosum, about 1 mg to about 15 mg of mixed Tocopherols, about 10 mg to about 200 mg of Magnolia officinalis, about 20 mg to about 100 mg of Piper methysticum, and about 1 mg to about 10 mg of Piper nigrum.

In some aspects, the compositions of the present disclosure comprise about 5 mg to about 80 mg of cannabidiol (CBD), about 1 mg to about 10 mg of cannabinol (CBN), about 40 mg to 260 mg of Ziziphus spinosa, about 30 mg to about 220 mg of Lycopus spp., about 30 mg to about 200 mg of Passiflora incarnata, about 10 mg to about 150 mg of Pueraria lobata, about 5 mg to about 40 mg of Actaea racemosa, about 10 mg to about 120 mg of Humulus lupulus, and about 1 mg to about 10 mg of Piper nigrum.

In certain aspects, the compositions of the present disclosure comprise about 5 mg to about 80 mg of cannabidiol (CBD), about 1 mg to about 15 mg of cannabinol (CBN), about 30 mg to about 220 mg of Lycopus spp., about 30 mg to about 200 mg of Passiflora incarnata, about 20 mg to about 100 mg of Lavendula officinalis, about 30 mg to about 120 mg of Scutellaria lateriflora, about 20 mg to about 100 mg of Piper methysticum, and about 1 mg to about 10 mg of Piper nigrum.

In certain aspects, the compositions of the present disclosure comprise about 5 mg to about 80 mg of cannabidiol (CBD), about 1 mg to about 30 mg of cannabigerol (CBG), about 40 mg to 260 mg of Ziziphus spinosa, about 0.1 mg to 4 mg of melatonin, about 10 mg to about 150 mg of Pueraria lobata, about 30 mg to about 250 mg of Matricaria recutita, about 10 mg to about 200 mg of Magnolia officinalis, and about 1 mg to about 10 mg of Piper nigrum.

In some aspects, the compositions of the present disclosure comprise about 5 mg to about 80 mg of cannabidiol (CBD), about 1 mg to about 30 mg of cannabigerol (CBG), about 50 mg to about 200 mg of Withania somnifera, about 30 mg to about 250 mg of Matricaria recutita, about 40 mg to 260 mg of Ziziphus spinosa, about 50 mg to about 200 mg of Polygala tenuifolia, about 0.1 mg to 4 mg of melatonin, about 15 mg to about 80 mg of Citrus reticulata (e.g., Citrus reticulata Blanco essential oil), and about 1 mg to about 10 mg of Piper nigrum.

In some aspects, the compositions of the present disclosure comprise about 5 mg to about 80 mg of cannabidiol (CBD), about 1 mg to about 15 mg of cannabinol (CBN), about 0.1 mg to 4 mg of melatonin, about 15 mg to about 80 mg of Citrus reticulata (e.g., Citrus reticulata Blanco essential oil), about 30 mg to about 150 mg of EPA/DHA, about 50 mg to about 200 mg of Withania somnifera, about 5 mg to about 50 mg of Cymbopogon flexuosus essential oil, about 10 mg to about 120 mg of Humulus lupulus, about 50 mg to about 200 mg of Grindelia spp., about 1 mg to about 15 mg of mixed Tocopherols, and about 1 mg to about 10 mg of Piper nigrum.

In certain aspects, the compositions of the present disclosure comprise about 5 mg to about 80 mg of cannabidiol (CBD), about 1 mg to about 15 mg of cannabinol (CBN), about 0.1 mg to 4 mg of melatonin, about 15 mg to about 70 mg of Anemarrhena asphodeloides, about 30 mg to about 120 mg of phosphatidylserine, about 15 mg to about 70 mg of alpha lipoic acid, about 40 mg to about 150 mg of silymarin, about 100 mg to about 280 mg of Schisandra chinensis, about 1 mg to about 80 mg of Citrus reticulata (e.g., Citrus reticulata Blanco essential oil), and about 1 mg to about 10 mg of Piper nigrum.

In certain aspects, the compositions of the present disclosure comprise about 5 mg to about 80 mg of cannabidiol (CBD), about 1 mg to about 15 mg of cannabinol (CBN), about 5 mg to about 50 mg of 5-HTP, about 40 mg to 260 mg of Ziziphus spinosa, about 50 mg to about 200 mg of Poria cocos, about 30 mg to about 150 mg of Ligusticum wallichii, about 5 mg to about 70 mg of Sceletium tortuosum, and about 1 mg to about 10 mg of Piper nigrum.

In certain aspects, the compositions of the present disclosure comprise about 5 mg to about 80 mg of cannabidiol (CBD), about 1 mg to about 15 mg of cannabinol (CBN), about 50 mg to about 200 mg of Poria cocos, about 0.1 mg to 4 mg of melatonin, about 50 mg to about 200 mg of Polygala tenuifolia, about 15 mg to about 150 mg of Panax ginseng, about 20 mg to about 100 mg of Rehmannia glutinosa, about 15 mg to about 70 mg of paeonol, and about 1 mg to about 10 mg of Piper nigrum.

One aspect of the present disclosure is directed to the methods for improving sleep quality comprising administering a composition comprising:
(i) about 5 mg to about 80 mg of cannabidiol (CBD) and about 1 mg to about 15 mg of cannabinol (CBN);
(ii) one or more of the following: about 40 mg to 260 mg of Ziziphus spinosa, about 0.1 mg to 4 mg of melatonin, and about 30 mg to about 200 mg of Passiflora incarnata; and
(iii) three or more of the following: about 30 mg to about 150 mg of eicosapentaenoic acid (EPA)/docosahexaenoic acid (DHA), about 1 mg to about 80 mg of Citrus reticulata (e.g., Citrus reticulata Blanco essential oil), about 50 mg to about 200 mg of Withania somnifera, about 30 mg to about 250 mg of Matricaria recutita, about 30 mg to about 220 mg of Lycopus spp., about 50 mg to about 200 mg of Poria cocos, about 5 mg to about 40 mg of Actaea racemosa, about 10 mg to about 150 mg of Pueraria lobata, about 15 mg to about 70 mg of Anemarrhena asphodeloides, about 30 mg to about 120 mg of phosphatidylserine, about 15 mg to about 70 mg of alpha lipoic acid, about 40 mg to about 150 mg of silymarin, about 100 mg to about 280 mg of Schisandra chinensis, about 5 mg to about 50 mg of Cymbopogon flexuosus essential oil, about 10 mg to about 120 mg of Humulus lupulus, about 50 mg to about 200 mg of Grindelia spp., about 30 mg to about 150 mg of Ligusticum wallichii, about 5 mg to about 50 mg of 5-hydroxytryptophan (5-HTP), about 15 mg to about 70 mg of paeonol, about 15 mg to about 150 mg of Panax ginseng, about 20 mg to about 100 mg of Rehmannia glutinosa, about 50 mg to about 200 mg of Polygala tenuifolia, about 20 mg to about 100 mg of Lavendula officinalis, about 50 mg to about 200 mg of Scutellaria lateriflora, about 50 mg to about 200 mg of Polygala tenuifolia, about 10 mg to about 150 mg of Pueraria lobata, about 5 mg to about 70 mg of Sceletium tortuosum, about 10 mg to about 80 mg of Paeonia lactiflora, and about 1 mg to about 10 mg of Piper nigrum.

In certain aspects, the improved sleep quality is sleep onset latency. In some aspects, the improved sleep quality is sleep duration. In certain aspects, the sleep onset latency is shortened. In certain aspects, the sleep duration is extended.

In certain aspects, the compositions of the present disclosure comprise:
(i) about 5 mg to about 80 mg of cannabidiol (CBD), about 1 mg to about 30 mg of cannabinol (CBN), and about 1 mg to about 10 mg of Piper nigrum; and
(ii) three or more of the following: about 30 mg to about 180 mg of eicosapentaenoic acid (EPA)/docosahexaenoic acid (DHA), about 80 mg to about 300 mg of Curcuma longa, about 30 mg to about 180 mg of Nigella sativa oil, about 1 mg to about 30 mg of Zanthoxylum spp., about 10 mg to about 80 mg of Citrus reticulata (e.g., Citrus reticulata Blanco essential oil), about 1 mg to about 15 mg of mixed Tocopherols, about 10 mg to about 80 mg of luteolin, about 0.001 mg to about 1 mg of vitamin D, about 150 mg to about 450 mg of Withania somnifera, about 50 mg to about 220 mg of Corydalis yanhusuo, about 1 mg to about 30 mg of Glycyrrhiza glabra, about 1 mg to about 30 mg of Lavandula angustifolia, about 150 mg to about 400 mg of Scutellaria lateriflora, about 30 mg to about 120 mg of coenzyme Q10, about 50 mg to about 250 mg of Angelica sinensis, about 50 mg to about 200 mg of Ligusticum chuanxiong, about 100 mg to about 300 mg of Salix alba, about 1 mg to about 15 mg of zinc, about 100 mg to about 350 mg of Commiphora mukul, about 0.01 mg to about 1 mg of selenium, about 0.001 mg to about 1 mg of iodine, about 10 mg to about 100 mg of Pueraria lobata, about 100 mg to about 350 mg of Viburnum prunifolium, about 50 mg to about 200 mg of Matricaria recutita about 1 mg to about 15 mg of Lobelia inflata, about 10 mg to about 80 mg of Melissa officinalis, about 10 mg to about 80 mg of Dioscorea villosa, about 10 mg to about 80 mg of Paeonia lactiflora, and about 1 mg to about 200 mg of Zingiber officinalis.

In some aspects, the compositions of the present disclosure comprise about 5 mg to about 80 mg of cannabidiol (CBD), about 1 mg to about 30 mg of cannabinol (CBN), about 1 mg to about 10 mg of Piper nigrum, about 30 mg to about 180 mg of eicosapentaenoic acid (EPA)/docosahexaenoic acid (DHA), about 80 mg to about 300 mg of Curcuma longa, about 30 mg to about 180 mg of Nigella sativa oil, about 1 mg to about 30 mg of Zanthoxylum spp., about 10 mg to about 80 mg of Citrus reticulata (e.g., Citrus reticulata Blanco essential oil), and about 1 mg to about 15 mg of mixed Tocopherols.

In certain aspects, the compositions of the present disclosure comprise about 5 mg to about 80 mg of cannabidiol (CBD), about 1 mg to about 30 mg of cannabinol (CBN), about 1 mg to about 10 mg of Piper nigrum, about 30 mg to about 180 mg of eicosapentaenoic acid (EPA)/docosahexaenoic acid (DHA), about 80 mg to about 300 mg of Curcuma longa, about 30 mg to about 180 mg of Nigella sativa oil, about 1 mg to about 30 mg of Zanthoxylum spp., about 10 mg to about 80 mg of Citrus reticulata (e.g., Citrus reticulata Blanco essential oil), about 1 mg to about 15 mg of mixed Tocopherols, about 10 mg to about 80 mg of luteolin, and about 0.001 mg to about 1 mg of vitamin D.

In some aspects, the compositions of the present disclosure comprise about 5 mg to about 80 mg of cannabidiol (CBD), about 1 mg to about 30 mg of cannabinol (CBN), about 1 mg to about 10 mg of Piper nigrum, about 150 mg to about 450 mg of Withania somnifera, about 50 mg to about 220 mg of Corydalis yanhusuo, and about 1 mg to about 30 mg of Glycyrrhiza glabra.

In certain aspects, the compositions of the present disclosure comprise about 5 mg to about 80 mg of cannabidiol (CBD), about 1 mg to about 30 mg of cannabinol (CBN), about 1 mg to about 10 mg of Piper nigrum, about 1 mg to about 30 mg of Lavandula angustifolia, about 1 mg to about 200 mg of Zingiber officinalis, about 150 mg to about 400 mg of Scutellaria lateriflora, and about 30 mg to about 120 mg of coenzyme Q10.

In some aspects, the compositions of the present disclosure comprise about 5 mg to about 80 mg of cannabidiol (CBD), about 1 mg to about 30 mg of cannabinol (CBN), about 1 mg to about 10 mg of Piper nigrum, about 10 mg to about 80 mg of luteolin, about 50 mg to about 250 mg of Angelica sinensis, about 50 mg to about 200 mg of Ligusticum chuanxiong, and about 50 mg to about 220 mg of Corydalis yanhusuo.

In certain aspects, the compositions of the present disclosure comprise about 5 mg to about 80 mg of cannabidiol (CBD), about 1 mg to about 30 mg of cannabinol (CBN), about 1 mg to about 10 mg of Piper nigrum, about 100 mg to about 300 mg of Salix alba, about 1 mg to about 15 mg of zinc, about 0.001 mg to about 1 mg of vitamin D, about 100 mg to about 350 mg of Commiphora mukul, about 0.01 mg to about 1 mg of selenium, and about 0.001 mg to about 1 mg of iodine.

In some aspects, the compositions of the present disclosure comprise about 5 mg to about 80 mg of cannabidiol (CBD), about 1 mg to about 30 mg of cannabinol (CBN), about 1 mg to about 10 mg of Piper nigrum, about 100 mg to about 300 mg of Salix alba, about 1 mg to about 15 mg of zinc, about 0.001 mg to about 1 mg of vitamin D, about 100 mg to about 350 mg of Commiphora mukul, about 0.01 mg to about 1 mg of selenium, about 0.001 mg to about 1 mg of iodine, and about 10 mg to about 100 mg of Pueraria lobata.

In certain aspects, the compositions of the present disclosure comprise about 5 mg to about 80 mg of cannabidiol (CBD), about 1 mg to about 30 mg of cannabinol (CBN), about 1 mg to about 10 mg of Piper nigrum, about 100 mg to about 350 mg of Viburnum prunifolium, about 50 mg to about 200 mg of Matricaria recutita, about 10 mg to about 80 mg of Paeonia lactiflora, about 10 mg to about 80 mg of Melissa officinalis, about 10 mg to about 80 mg of Dioscorea villosa, and about 1 mg to about 200 mg of Zingiber officinalis.

One aspect of the present disclosure is directed to the methods for relieving pain in a subject, comprising administering a composition comprising:
(i) about 5 mg to about 80 mg of cannabidiol (CBD), about 1 mg to about 30 mg of cannabinol (CBN), about 1 mg to about 30 mg of cannabigerol (CBG), or any combinations thereof, and about 1 mg to about 10 mg of Piper nigrum; and
(ii) three or more of the following: about 30 mg to about 180 mg of eicosapentaenoic acid (EPA)/docosahexaenoic acid (DHA), about 80 mg to about 300 mg of Curcuma longa, about 30 mg to about 180 mg of Nigella sativa oil, about 1 mg to about 30 mg of Zanthoxylum spp., about 10 mg to about 80 mg of Citrus reticulata (e.g., Citrus reticulata Blanco essential oil), about 1 mg to about 15 mg of mixed Tocopherols, about 10 mg to about 80 mg of luteolin, about 0.001 mg to about 1 mg of vitamin D, about 150 mg to about 450 mg of Withania somnifera, about 50 mg to about 220 mg of Corydalis yanhusuo, about 1 mg to about 30 mg of Glycyrrhiza glabra, about 1 mg to about 30 mg of Lavandula angustifolia, about 150 mg to about 400 mg of Scutellaria lateriflora, about 30 mg to about 120 mg of coenzyme Q10, about 50 mg to about 250 mg of Angelica sinensis, about 50 mg to about 200 mg of Ligusticum chuanxiong, about 100 mg to about 300 mg of Salix alba, about 1 mg to about 15 mg of zinc, about 100 mg to about 350 mg of Commiphora mukul, about 0.01 mg to about 1 mg of selenium, about 0.001 mg to about 1 mg of iodine, about 10 mg to about 100 mg of Pueraria lobata, about 100 mg to about 350 mg of Viburnum prunifolium, about 50 mg to about 200 mg of Matricaria recutita, about 1 mg to about 15 mg of Lobelia inflata, about 10 mg to about 80 mg of Melissa officinalis, about 10 mg to about 80 mg of Dioscorea villosa, about 10 mg to about 200 mg of Magnolia officinalis, about 20 mg to about 100 mg of Piper methysticum, about 10 mg to about 250 mg of Polygala tenuifolia, and about 1 mg to about 200 mg of Zingiber officinalis.

In some aspects, pain comprises musculoskeletal pain, pain induced by metabolic syndrome, autoimmune pain, chronic pain, diabetic neuropathy pain, polycystic ovary syndrome pain, endometriosis pain, pain induced by cancer, pain induced by inflammation, general pain, pain induced by migraines, pain induced by anxiety, pelvic pain, fibromyalgia pain, pain induced by hypothyroidism, or any combination thereof.

In certain aspects, the compositions of the present disclosure comprise:
(i) about 5 mg to about 80 mg of cannabidiol (CBD), about 1 mg to about 30 mg of cannabinol (CBN), about 1 mg to about 30 mg of cannabigerol (CBG), or any combinations thereof;
(ii) one or more of the following: about 1 mg to about 10 mg of Piper nigrum and about 10 mg to about 150 mg of Citrus reticulata (e.g., Citrus reticulata Blanco essential oil); and
(iii) two or more of the following: about 10 mg to about 200 mg Lavandula officinalis, about 10 mg to about 250 mg of Polygala tenuifolia, about 100 mg to about 450 mg of Withania somnifera, about 30 mg to about 150 mg of rutaecarpine, about 30 mg to about 150 mg of Passiflora incarnata, about 100 mg to about 400 mg of Ganoderma lucidum, about 50 mg to about 250 mg of Matricaria recutita, about 10 mg to about 150 mg of Pueraria lobata, about 40 mg to 300 mg of Ziziphus spinosa, about 5 mg to about 100 mg of Sceletium tortuosum, about 5 mg to about 50 mg of Crocus sativus, about 100 mg to about 350 mg of Schisandra chinensis, about 80 mg to about 300 mg of dihydromyricetin, about 5 mg to about 80 mg of 5-hydroxytryptophan (5-HTP), about 50 mg to about 300 mg of Poria cocos, about 20 mg to about 200 mg of Panax ginseng, about 100 mg to about 400 mg of Centella asiatica, about 10 mg to about 200 mg of Magnolia officinalis, about 20 mg to about 100 mg of Piper methysticum, and about 30 mg to about 150 mg of paeonol.

In some aspects, the compositions of the present disclosure comprise:
(i) about 5 mg to about 80 mg of cannabidiol (CBD) and about 1 mg to about 30 mg of cannabinol (CBN), and/or about 1 mg to about 30 mg of cannabigerol (CBG), or any combinations thereof, and
(ii) three or more of the following: about 1 mg to about 10 mg of Piper nigrum about 10 mg to about 150 mg of Citrus reticulata (e.g., Citrus reticulata Blanco essential oil), about 10 mg to about 200 mg Lavandula officinalis, about 10 mg to about 250 mg of Polygala tenuifolia, about 100 mg to about 450 mg of Withania somnifera, about 30 mg to about 150 mg of rutaecarpine, about 30 mg to about 150 mg of Passiflora incarnata, about 100 mg to about 400 mg of Ganoderma lucidum about 50 mg to about 250 mg of Matricaria recutita, about 10 mg to about 150 mg of Pueraria lobata, about 40 mg to 300 mg of Ziziphus spinosa, about 5 mg to about 100 mg of Sceletium tortuosum, about 5 mg to about 50 mg of Crocus sativus, about 100 mg to about 350 mg of Schisandra chinensis, about 80 mg to about 300 mg of dihydromyricetin, about 5 mg to about 80 mg of 5-hydroxytryptophan (5-HTP), about 50 mg to about 300 mg of Poria cocos, about 20 mg to about 200 mg of Panax ginseng, about 100 mg to about 400 mg of Centella asiatica, about 10 mg to about 200 mg of Magnolia officinalis, about 20 mg to about 100 mg of Piper methysticum, and about 30 mg to about 150 mg of paeonol.

In certain aspects, the compositions of the present disclosure comprise about 5 mg to about 80 mg of cannabidiol (CBD), about 1 mg to about 30 mg of cannabinol (CBN), about 1 mg to about 10 mg of Piper nigrum, about 10 mg to about 200 mg Lavandula officinalis, about 20 mg to about 100 mg of Piper methysticum, and about 100 mg to about 450 mg of Withania somnifera.

In some aspects, the compositions of the present disclosure comprise about 5 mg to about 80 mg of cannabidiol (CBD), about 1 mg to about 30 mg of cannabinol (CBN), about 1 mg to about 10 mg of Piper nigrum, about 30 mg to about 150 mg of rutaecarpine, about 30 mg to about 150 mg of Passiflora incarnata, about 20 mg to about 100 mg of Piper methysticum, and about 100 mg to about 400 mg of Ganoderma lucidum.

In certain aspects, the compositions of the present disclosure comprise about 5 mg to about 80 mg of cannabidiol (CBD), about 1 mg to about 30 mg of cannabinol (CBN), about 1 mg to about 30 mg of cannabigerol (CBG), about 50 mg to about 250 mg of Matricaria recutita, about 10 mg to about 150 mg of Pueraria lobata, about 40 mg to 300 mg of Ziziphus spinosa, about 5 mg to about 100 mg of Sceletium tortuosum, and about 10 mg to about 150 mg of Citrus reticulata (e.g., Citrus reticulata Blanco essential oil).

In some aspects, the compositions of the present disclosure comprise about 5 mg to about 80 mg of cannabidiol (CBD), about 1 mg to about 30 mg of cannabigerol (CBG), about 100 mg to about 450 mg of Withania somnifera, about 5 mg to about 50 mg of Crocus sativus, about 10 mg to about 250 mg of Polygala tenuifolia, and about 10 mg to about 150 mg of Citrus reticulata (e.g., Citrus reticulata Blanco essential oil).

In certain aspects, the compositions of the present disclosure comprise about 5 mg to about 80 mg of cannabidiol (CBD), about 1 mg to about 30 mg of cannabigerol (CBG), about 1 mg to about 10 mg of Piper nigrum, about 100 mg to about 350 mg of Schisandra chinensis, and about 80 mg to about 300 mg of dihydromyricetin.

In some aspects, the compositions of the present disclosure comprise about 5 mg to about 80 mg of cannabidiol (CBD), about 1 mg to about 30 mg of cannabinol (CBN), about 1 mg to about 10 mg of Piper nigrum, about 5 mg to about 80 mg of 5-hydroxytryptophan (5-HTP), about 40 mg to 300 mg of Ziziphus spinosa, and about 50 mg to about 300 mg of Poria cocos.

In certain aspects, the compositions of the present disclosure comprise about 5 mg to about 80 mg of cannabidiol (CBD), about 1 mg to about 30 mg of cannabinol (CBN), about 1 mg to about 30 mg of cannabigerol (CBG), about 1 mg to about 10 mg of Piper nigrum, about 20 mg to about 200 mg of Panax ginseng, about 10 mg to about 150 mg of Pueraria lobata, and about 100 mg to about 400 mg of Centella asiatica.

In some aspects, the compositions of the present disclosure comprise about 5 mg to about 80 mg of cannabidiol (CBD), about 1 mg to about 30 mg of cannabinol (CBN), about 1 mg to about 30 mg of cannabigerol (CBG), about 1 mg to about 10 mg of Piper nigrum, about 20 mg to about 200 mg of Panax ginseng, about 100 mg to about 400 mg of Ganoderma lucidum, and about 30 mg to about 150 mg of paeonol.

One aspect of the present disclosure is directed to the methods for improving anxiety symptoms in a subject, comprising administering a composition comprising:
(i) about 5 mg to about 80 mg of cannabidiol (CBD), about 1 mg to about 30 mg of cannabinol (CBN), about 1 mg to about 30 mg of cannabigerol (CBG), or any combinations thereof;
(ii) one or more of the following: about 1 mg to about 10 mg of Piper nigrum and about 10 mg to about 150 mg of Citrus reticulata (e.g., Citrus reticulata Blanco essential oil); and
(iii) two or more of the following: about 10 mg to about 200 mg Lavandula officinalis, about 10 mg to about 250 mg of Polygala tenuifolia, about 100 mg to about 450 mg of Withania somnifera, about 30 mg to about 150 mg of rutaecarpine, about 30 mg to about 150 mg of Passiflora incarnata, about 100 mg to about 400 mg of Ganoderma lucidum, about 50 mg to about 250 mg of Matricaria recutita, about 10 mg to about 150 mg of Pueraria lobata, about 40 mg to 300 mg of Ziziphus spinosa, about 5 mg to about 100 mg of Sceletium tortuosum, about 5 mg to about 50 mg of Crocus sativus, about 100 mg to about 350 mg of Schisandra chinensis, about 80 mg to about 300 mg of dihydromyricetin, about 5 mg to about 80 mg of 5-hydroxytryptophan (5-HTP), about 50 mg to about 300 mg of Poria cocos, about 20 mg to about 200 mg of Panax ginseng, about 100 mg to about 400 mg of Centella asiatica, about 10 mg to about 200 mg of Magnolia officinalis, about 20 mg to about 100 mg of Piper methysticum, and about 30 mg to about 150 mg of paeonol.

In some aspects, the compositions of the present disclosure further comprise about 5 mg to about 175 mg of medium-chain triglyceride (MCT) oil, about 5 mg to about 100 mg of beeswax (e.g., yellow USP), about 1 mg to about 30 mg sunflower lecithin, about 0 mg to about 30 mg glycerin (e.g., 99.6%, USP, vegetable-based), or any combination thereof.

In certain aspects, anxiety comprises social anxiety disorder, anxiety due to caffeine excess, generalized anxiety disorder, anxiety in alcoholism, post-traumatic stress disorder (PTSD), anxiety in the elderly, anxiety induced depression, a specific phobia, obsessive-compulsive disorder, or any combination thereof.

In certain aspects, anxiety symptoms comprise excessive worrying, feeling agitated, restlessness, difficulty concentrating, irritability, tense muscles, trouble falling or staying asleep, panic attacks, irrational fears, avoiding social situations, or any combination thereof.

In certain aspects, the compositions of the present disclosure are formulated for oral delivery. In certain aspects, the compositions of the present disclosure are formulated as a solid dosage form. In some aspects, the solid dosage form is a capsular dosage form. In certain aspects, the capsular dosage form comprises gelatin material.

One aspect of the present disclosure is directed to the methods for improving sleep quality, relieving pain and/or improving anxiety symptoms in a subject, comprising administering one or more of the compositions of the present disclosure.

### BRIEF DESCRIPTION OF THE FIGURES

FIG. 1 shows the ingredients and targets of sleep formulation S10 designed to address the sleep challenges of aging consumers (e.g., older women suffering symptoms of menopause such as hot flashes and night sweats).

FIG. 2 shows the ingredients and targets of sleep formulation S12 designed to address the sleep challenges for people who have difficulty falling asleep due to stress, restlessness, or low levels of anxiety.

FIG. 3 shows the ingredients and targets of sleep formulation S14 designed to address the sleep challenges for people who have difficulty staying asleep due to anxiety or mild depression.

FIG. 4 shows the ingredients and targets of sleep formulation S16 designed to address the sleep challenges for people who have difficulty staying asleep due to anxiety or mild depression.

FIG. 5 shows the ingredients and targets of sleep formulation S18 designed to address the sleep challenges for people who have poor concentration, forgetfulness, malaise, confusion, and obesity.

FIG. 6 shows the ingredients and targets of sleep formulation S20 designed to address the sleep challenges of older consumers.

FIG. 7 shows the ingredients and targets of anxiety formulation C30 designed to address the challenges for people with social anxiety disorder.

FIG. 8 shows the ingredients and targets of anxiety formulation C32 designed to address the challenges for people with caffeine-induced anxiety.

FIG. 9 shows the ingredients and targets of anxiety formulation C34 designed to address the challenges for females with generalized anxiety disorder.

FIG. 10 shows the ingredients and targets of anxiety formulation C36 designed to address the challenges for people with anxiety-induced depression.

FIG. 11 shows the ingredients and targets of anxiety formulation C38 designed to address the challenges for people with alcoholism-induced anxiety.

FIG. 12 shows the ingredients and targets of anxiety formulation C40 designed to address the challenges for anxiety for aged males.

FIG. 13 shows the ingredients and targets of pain formulation P50 designed to address the challenges for males suffering from musculoskeletal pain, metabolic syndrome (MetS) autoimmune pain, chronic pain, and/or diabetic neuropathy.

FIG. 14 shows the ingredients and targets of pain formulation P52 designed to address the challenges for females suffering from female polycystic ovary syndrome (PCOS), MetS, endometriosis, musculoskeletal pain, autoimmune pain, chronic pain, and/or diabetic neuropathy.

FIG. 15 shows the ingredients and targets of pain formulation P54 designed to address the challenges for people suffering from pain induced by cancer, inflammation, and/or generalized pain.

FIG. 16 shows the ingredients and targets of pain formulation P58 designed to address the challenges for people with endometriosis, pelvic pain, and/or fibromyalgia.

FIG. 17 shows the ingredients and targets of pain formulation P62 designed to address the challenges for hypothyroid females (e.g., uterine spasm).

FIG. 18A shows the quality of sleep before consumers began taking only sleep formulations of the present disclosure, as illustrated by percentage (%) of low quality sleep, extremely low quality sleep, good quality sleep, and extremely good quality sleep. The first number represents the actual number of consumers and the second number represents the percentage (%).

FIG. 18B shows the quality of sleep while consumers were taking only sleep formulations of the present disclosure, as illustrated by percentage (%) of low quality sleep, extremely low quality sleep, good quality sleep, and extremely good quality sleep measured in Trial #1 and Trial #2. The first number represents the actual number of consumers and the second number represents the percentage (%).

FIG. 18C shows the quality of sleep while consumers were taking only sleep formulations of the present disclosure, as illustrated by percentage (%) of low quality sleep, extremely low quality sleep, good quality sleep, and extremely good quality sleep represented as composite results from Trial #1 and Trial #2. The first number represents the actual number of consumers and the second number represents the percentage (%).

FIG. 18D shows the consumers' ratings of the following aspects of their sleep experience while taking sleep formulations of the present disclosure: time needed to fall asleep; ability to stay asleep; ability to return to sleep; and morning energy level.

FIG. 19A shows stress levels before consumers began taking only anxiety formulations of the present disclosure, as illustrated by percentage (%) of high stress/anxiousness levels, extremely high stress/anxiousness levels, low stress/anxiousness levels, and extremely low stress/anxiousness levels. The first number represents the actual number of consumers and the second number represents the percentage (%).

FIG. 19B shows stress levels while consumers were taking only anxiety formulations of the present disclosure, as illustrated by percentage (%) of high stress/anxiousness levels, extremely high stress/anxiousness levels, low stress/anxiousness levels, and extremely low stress/anxiousness levels measured in Trial #1 and Trial #2. The first number represents the actual number of consumers and the second number represents the percentage (%).

FIG. 19C shows stress levels while consumers were taking only anxiety formulations of the present disclosure, as illustrated by percentage (%) of high stress/anxiousness levels, extremely high stress/anxiousness levels, low stress/anxiousness levels, and extremely low stress/anxiousness levels represented as composite results from Trial #1 and Trial #2. The first number represents the actual number of consumers and the second number represents the percentage (%).

FIG. 19D shows the consumers' ratings of the following aspects of their anxiety experience while taking anxiety formulations of the present disclosure: overall stress/anxiousness; ability to focus; restlessness; sadness; and anger or irritability.

FIG. 20A shows the quality of sleep before consumers began taking both sleep and anxiety formulations of the present disclosure, as illustrated by percentage (%) of low quality sleep, extremely low quality sleep, good quality sleep, and extremely good quality sleep. The first number represents the actual number of consumers and the second number represents the percentage (%).

FIG. 20B shows the quality of sleep while consumers were simultaneously taking both sleep and anxiety formulations of the present disclosure, as illustrated by percentage (%) of low quality sleep, extremely low quality sleep, good quality sleep, and extremely good quality sleep measured in Trial #1 and Trial #2. The first number represents the actual number of consumers and the second number represents the percentage (%).

FIG. 20C shows the quality of sleep while consumers were simultaneously taking both sleep and anxiety formulations of the present disclosure, as illustrated by percentage (%) of low quality sleep, extremely low quality sleep, good quality sleep, and extremely good quality sleep represented as composite results from Trial #1 and Trial #2.

FIG. 20D shows the consumers' ratings of the following aspects of their sleep experience while simultaneously taking both sleep and anxiety formulations of the present disclosure: time needed to fall asleep; ability to stay asleep; ability to return to sleep; and morning energy level.

FIG. 21A shows stress levels before consumers began taking both sleep and anxiety formulations of the present disclosure, as illustrated by percentage (%) of high stress/anxiousness levels, extremely high stress/anxiousness levels, low stress/anxiousness levels, and extremely low stress/anxiousness levels. The first number represents the actual number of consumers and the second number represents the percentage (%).

FIG. 21B shows stress levels while consumers were simultaneously taking both anxiety and sleep formulations of the present disclosure, as illustrated by percentage (%) of high stress/anxiousness levels, extremely high stress/anxiousness levels, low stress/anxiousness levels, and extremely low stress/anxiousness levels measured in Trial #1 and Trial #2. The first number represents the actual number of consumers and the second number represents the percentage (%).

FIG. 21C shows stress levels while consumers were simultaneously taking both anxiety and sleep formulations of the present disclosure, as illustrated by percentage (%) of high stress/anxiousness levels, extremely high stress/anxiousness levels, low stress/anxiousness levels, and extremely low stress/anxiousness levels represented as composite results from Trial #1 and Trial #2.

FIG. 21D shows the consumers' ratings of the following aspects of their anxiety experience while simultaneously taking both anxiety and sleep formulations of the present disclosure: overall stress/anxiousness; ability to focus; restlessness; sadness; and anger or irritability.

FIG. 22A shows the manufacturing process of assembling and encapsulating formulations of the present disclosure.

FIG. 22B shows sleep formulation S10 recipe sample.

### DETAILED DESCRIPTION OF THE DISCLOSURE

Described herein are cannabinoid therapy formulations for sleep, pain, and anxiety conditions. In particular, the present disclosure relates to the compositions comprising at least one negative allosteric modulator of the cannabinoid receptor 1 (CB1) (e.g., cannabidiol (CBD)), at least one partial agonist of CB1 (e.g., cannabinol (CBN)), and at least one gamma-aminobutyric acid (GABA)A receptor (GABAAR) activator (e.g., Ziziphus spinosa, Passiflora incarnata, Withania somnifera, and/or melatonin), and/or at least one transient receptor potential cation channel, subfamily A, member 1 (TRPA1) activator (e.g., Piper nigrum).

Before explaining aspects of the disclosure in detail, it is to be understood that the disclosure is not limited in its application to the details set forth in the following description or exemplified by the Examples. The disclosure is capable of other aspects or of being practiced or carried out in various ways. Also, it is to be understood that the phraseology and terminology employed herein is for the purpose of description and should not be regarded as limiting.

### I. General Definitions

In order that the present disclosure can be more readily understood, certain terms are first defined. As used in this application, except as otherwise expressly provided herein, each of the following terms shall have the meaning set forth below. Additional definitions are set forth throughout the application.

As used herein, the singular form “a”, “an” and “the” include plural references unless the context clearly dictates otherwise. For example, the term “an ingredient” or “at least one ingredient” can include a plurality of ingredients, including mixtures thereof.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless specified otherwise, all of the designations “A %-B %,” “A-B %,” “A % to B %,” “A to B %,” “A %-B,” “A % to B” are given their ordinary and customary meaning. In some aspects, these designations are synonyms.

It is understood that wherever aspects are described herein with the language “comprising,” otherwise analogous aspects described in terms of “consisting of” and/or “consisting essentially of” are also provided.

As used herein, the terms “comprises”, “comprising”, “includes”, “including”, “having,” and their conjugates mean “including but not limited to.”

As used herein, the term “consisting of” means “including and limited to.”

As used herein, the term “consisting essentially of” means the specified material of a composition, or the specified steps of a method, and those additional materials or steps that do not materially affect the basic characteristics of the material or method.

When the term “about” is used in conjunction with a numerical value or range, it modifies that value or range by extending the boundaries above and below the numerical values set forth. The term “about” is used herein to modify a numerical value above and below the stated value by a variance of 10 percent, up or down (higher or lower), i.e., 10%, unless a different variance is indicated (e.g., ±30%, ±20%, ±5%, +1%, etc.).

As used herein, the term “process” and “method” refer to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term a “composition” as described herein refers to a combination of an active ingredient (e.g., cannabis ingredient, such as CBD/CBN) with other ingredients (e.g., plant based botanicals, such as lavender, ginseng, and magnolia). The terms “composition” and “formulation” are used interchangeably herein

The terms “administer,” “administering,” “administration,” and the like, as used herein, refer to the administration of a composition (e.g., a composition as described herein) to a subject or system. Administration to an animal subject (e.g., to a human) can be by any appropriate route, such as one described herein.

The terms “subject” and “individual”, as included herein, are used interchangeably. None of the terms are to be interpreted as requiring the supervision of a medical professional (e.g., a doctor, nurse, physician's assistant, orderly, hospice worker).

The term “symptom” or “clinical symptom” refers to a subjective evidence of a disease, such as a feeling of nausea, as perceived by the subject.

Throughout this application, various aspects of this disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range, such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5 and 6. This applies regardless of the breadth of the range.

“Percent” or “%” as used herein refers to weight percentage (w/w) unless otherwise specified.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

It is appreciated that certain features of the disclosure, which are, for clarity, described in the context of separate aspects, can also be provided in combination in a single aspect. Conversely, various features of the disclosure, which are, for brevity, described in the context of a single aspect, can also be provided separately or in any suitable subcombination or as suitable in any other described aspect of the disclosure. Certain features described in the context of various aspects are not to be considered essential features of those aspects, unless the aspect is inoperative without those elements.

### II. Compositions for Sleep, Pain, and Anxiety Conditions

The present disclosure provides cannabinoid therapy formulations for sleep, pain, and anxiety conditions.

Cannabis is a genus of flowering plants that includes one species, i.e., Cannabis sativa. The subspecies of Cannabis sativa include Cannabis sativa subsp. sativa, Cannabis sativa subsp. indica, and Cannabis sativa subsp. ruderalis. See e.g., McPartland J M., Cannabis and Cannabinoid Research 3(1):203-212 (2018). The term “Cannabis plant(s)” encompasses wild type Cannabis and also variants thereof, including cannabis chemovars which naturally contain different amounts of the individual cannabinoids. For example, some Cannabis varietals have been bred to produce minimal levels of THC, the principal psychoactive constituent responsible for the high associated with it and other varietals have been selectively bred to produce high levels of THC and other psychoactive cannabinoids. See e.g., Gloss D., Neurotherapeutics., 12(4): 731-734 (2015).

Cannabis plants produce a unique family of terpeno-phenolic compounds called cannabinoids, which produce the “high” one experiences from consuming Cannabis. The two cannabinoids usually produced in greatest abundance are cannabidiol (CBD) and/or A9-tetrahydrocannabinol (A9-THC), but only THC is euphoric. Cannabis plants are categorized by their chemical phenotype or “chemotype,” based on the overall amount of THC produced, and on the ratio of THC to CBD. Although overall cannabinoid production is influenced by environmental factors, the THC/CBD ratio is genetically determined and remains fixed throughout the life of a plant. Non-drug plants produce relatively low levels of THC and high levels of CBD, while drug plants produce high levels of THC and low levels of CBD. The best studied cannabinoids include tetrahydrocannabinol (THC), cannabidiol (CBD) and cannabinol (CBN). Other cannabinoids include for example, cannabichromene (CBC), cannabigerol (CBG) cannabinidiol (CBND), Cannabicyclol (CBL), Cannabivarin (CBV), Tetrahydrocannabivarin (THCV), Cannabidivarin (CBDV), Cannabichromevarin (CBCV) Cannabigerovarin (CBGV), and Cannabigerol Monomethyl Ether (CBGM). See e.g., U.S. Pat. No. 9,730,911

Cannabinoids are derived from their respective 2-carboxylic acids (2-COOH) by decarboxylation (catalyzed by heat, light, or alkaline conditions). As a general rule, the carboxylic acids form of the cannabinoid have the function of a biosynthetic precursor. See e.g., U.S. Patent Application Publ. No. 2012/0046352. As used herein, THC, CBD, CBN, CBC, CBG, CBND, CBL, CBV, THCV, CBDV, CBCV, CBGV, and CBGM refer to the decarboxylated form of the cannabinoid. THCa, CBDa, CBNa, CBCa, CBGa, CBNDa, CBLa, CBVa, THCVa, CBDVa, CBCVa, CBGVa, and CBGM refer to the acid form of the cannabinoid.

Tetrahydrocannabinol (THC) is the primary psychoactive component of the Cannabis plant. THC is only psychoactive in is decarboxylated state. The carboxylic acid form (THCa) is non-psychoactive. Delta-9-tetrahydrocannabinol (A9-THC, THC) and delta-8-tetrahydrocannabinol (A8-THC), mimic the action of anandamide, a neurotransmitter produced naturally in the body. These two THCs produce the effects associated with cannabis by binding to the CB1 cannabinoid receptors in the brain. THC appears to ease moderate pain (analgesic) and to be neuroprotective, while also offering the potential to reduce neuroinflammation and to stimulate neurogenesis. THC has approximately equal affinity for the CB1 and CB2 receptors. See e.g., U.S. Pat. No. 9,730,911.

Unlike THC, cannabidiol (CBD) has no euphoric effects. CBD is a negative allosteric modulator of the cannabinoid receptor 1 (CB1). It can act as the antagonist/negative allosteric modulator of CB1, a weak inverse agonist of cannabinoid receptor 2 (CB2), G protein-coupled receptor (GPR) 55 antagonist, G-protein-coupled receptor 18 (GPR18) antagonist, and transient receptor potential channel of melastatin type 8 (TRPM8) antagonist. In addition, CBD can activate 5-HT_{1A/2A/3A} serotonergic receptors, transient receptor potential channels of vanilloid type 1 and 2 (TRPV1-2) and ankyrin type 1 (TRPA1), respectively, glycine receptor α-1 subunit and α-1-β subunit, and peroxisome proliferator-activated receptor (PPAR)γ. Furthermore, CBD can antagonize alpha-1 adrenergic, δ-opioid, and μ-opioid receptors, inhibit synaptosomal uptake of noradrenaline, dopamine, serotonin and GABA, cellular uptake of anandamide, and adenosine uptake, act on mitochondria Ca²⁺ stores, block low-voltage-activated (T-type) Ca²⁺ channels, and/or inhibit activity of fatty amide hydrolase (FAAH). CBD is also an allosteric modulator of glycine receptor α-1 subunit and α-1-β subunit and δ-opioid, and μ-opioid receptors, 15-lipoxygenase inhibitor, phospholipase A2 modulator, and CBD can positively allosterically modulate cyclooxygenases (COX) 1 and 2. See e.g., Ibeas Bih, C., et al., Neurotherapeutics, 12:699-730 (2015), which is incorporated herein by reference in its entirety.

Cannabinol (CBN) is the primary product of THC degradation. CBN content increases as THC degrades in storage, and with exposure to light and air. It is only mildly psychoactive. It can act as a partial agonist (binds directly to the receptor but induces much lower effects compared to a full agonist) of CB1 and an agonist to TRPV2 receptor. CBN can also activate CB2 receptor and antagonize cellular uptake of anandamide. See e.g., Karniol I G., et al., Pharmacology, 13 (6):502-512 (1975); McCallum N D., et al., Experientia., 31(5): 520-521 (1975); Pertwee R G, Br J Pharmacol 153(2):199-215 (2008).

Cannabigerol (CBG) is non-psychotomimetic, but can still affect the overall effects of Cannabis. It can act as an α2-adrenergic receptor agonist, 5-HT1A receptor antagonist, and CB1 receptor antagonist. It can also bind to the CB2 receptor. See e.g., Navarro G., et al., Front Pharmacol. 9:632. (published Jun. 21, 2018); Cascio M. G. et al., Br. J. Pharmacol., 159:129-141 (2010).

Tetrahydrocannabivarin (THCV) is prevalent in certain central Asian and southern African strains of Cannabis. It is an agonist at lower doses and antagonist at higher doses of THC at CB1 receptors and can attenuate the psychoactive effects of THC. Cannabidivarin (CBDV) is usually a minor constituent of the cannabinoid profile. Cannabichromene (CBC) is non-psychoactive and does not affect the euphoric activity of THC. More common in tropical cannabis varieties. Its effects can include anti-inflammatory and analgesic effects. See e.g., U.S. Pat. No. 9,730,911.

Cannabis products have side effects from the tetrahydrocannabinol (THC) contained within these products. In certain aspects, the compositions of the present disclosure comprise lower doses of THC/CBN. In certain aspects, the compositions of the present disclosure do not comprise THC. To increase efficacy, amplify molecular outcomes, and offset the potential adverse effects of cannabinoids, the compositions of the present disclosure are constructed using different strategies described herein.

The endocannabinoid system (ECS) plays an important role in different strategies used to construe the compositions of the present disclosure. The ECS is targeted not only by cannabinoids from Cannabis sativa, but also other medicinal plants (e.g. Piper nigrum, Sceletium tortuosum etc.)

In some aspects, the compositions of the present disclosure are prepared by combining of eastern and western philosophies/remedies and botanicals/herbs to target multiple targets on the ECS and other enzymes and receptors that help control sleep, pain, and anxiety. Eastern medicine is a time-tested perspective that has provided novel active compounds that have become pharmaceuticals from their medicinal plants (e.g., reserpine, an alkaloid derived from the plant Rauwolfia; forskolin, a chemical found in the roots of the plant Plectranthus barbatus (Coleus forskohlii); and artemisinin, the sesquiterpene lactone produced by the plant Artemisia annua).

In some aspects, the compositions of the present disclosure are prepared by applying a model of constructing complex phytochemical matrices that include synergist and triggering biochemical pathways that converge on a pharmacological outcome.

In some aspects, the compositions of the present disclosure were informed by clinical data by using a physician's database of thousands of patients.

In some aspects, the compositions of the present disclosure are prepared by applying an overlapping pharmacological pleiotropy, which is also a strategy used by traditional medicine. As used herein, the term “pharmacological pleiotropy” refers to a plant compound having multiple targets and thus, multiple effects on physiology.

In some aspects, the compositions of the present disclosure are prepared by applying precision medicine. Precision medicine, an individualized approach to physiological patterns, is harnessed through the use of algorithms to determine which specific formula will best match the needs of the consumer.

In some aspects, the compositions of the present disclosure are prepared by applying network pharmacology. Network pharmacology is precisely applied to construct each formula of the present disclosure through deliberately targeting multiple, but select, pharmacological targets. By careful construction of each formula, lower doses are needed for each target, any potential for adverse events is reduced, and desired pharmacological outcomes are amplified. Through a novel combination of all the approaches, e.g., as described herein, the Precision Network Pharmacology (PNP) model is generated.

In addition to cannabinoids, cannabis plants produce terpenes, a diverse group of organic hydrocarbons that are the building blocks of the cannabinoids.

Over 100 different terpenes have been identified in the cannabis plant, and every strain tends toward a unique terpene type and composition. The terpenes act synergistically with the cannabinoids to provide a therapeutic effect. The following are examples of some common terpenes found in Cannabis.

β-Myrcene—can be used to treat insomnia and pain. It is unique because it allows chemicals to cross the blood-brain barrier more easily, allowing for cannabinoids to have a faster onset. Myrcene can also increase the psychoactive effect of THC, making for a more intense cerebral high. Myrcene is naturally occurring in lemongrass, thyme, and hops. It possesses antimicrobial, antiseptic, analgesic, antioxidant, anti-carcinogen, anti depressant, anti-inflammatory, and muscle relaxing effects. See e.g., Do Vale, T G. et al., Phytomedicine., 9(8):709-714 (2002); Rao, V S. et al., Journal of Pharmacy and Pharmacology, 42(12):877-888 (1990).

Linalool—has a delicate, floral aroma and is found in hundreds of different plants. Linalool is present in lavender, cinnamon, birch, and coriander. It can be used for sleep disorders and in treating depression and anxiety. See e.g., Guzmin-Gutierrez, S L. et al., Life Sciences, 1(128):24-29 (2015). Linalool can also be used as an analgesic. See e.g., de Cissia da Silveira E Sá et al., International Journal of Molecular Sciences, 18(12): 2392 (2017).

Limonene from Citrus spp. and Cannabis sativa—has an energizing, citrus scent. It can be used for combatting gastric reflux and heartburn. Limonene has antifungal, antimicrobial and anti-inflammatory properties. It can be powerful for elevating mood and reducing stress. See e.g., U.S. Pat. No. 9,730,911.

α-Pinene—has pine needles and rosemary scent and β-Pinene, which has dill, parsley, rosemary, basil, yarrow, rose, and hops like smell. Pinene can increase mental focus and energy, as well as act as an expectorant, bronchodilator, and topical antiseptic. See e.g., Falk-Filipsson, A. et al., Journal of Toxicology and Environmental Health, 38(1), 77-88 (1993). It also has anti-inflammatory effects (see e.g., Kim, D S., et al., The American Journal of Chinese Medicine, 43(4):731-42 (2015)) and antibiotic and gastro protectant abilities (see e.g., Pinheiro, M., et al., Pharmacognosy Magazine, 11(41):123-130 (2015)). It can easily cross the blood-brain barrier where it inhibits activity of acetylcholinesterase, which destroys acetylcholine, an information transfer molecule, resulting in better memory. It can counteract THC's activity, which can lead to low acetylcholine levels. See e.g., U.S. Pat. No. 9,730,911.

Humulene—found in abundance in its Humulus lupulus, also known as common hops. It is present in ginseng, sage, clove, and basil. Used in ancient Chinese medicine, humulene can be an effective appetite suppressant. It has antibacterial, anti-inflammatory and antitumor properties. See e.g., Fernandes E S. et al., Eur J Pharmacol., 569(3):228-236 (2007).

Borneol—has menthol, camphor, pine, and woody scent. Can be easily converted into menthol. It is considered a “calming sedative” in Chinese medicine. It can be used for fatigue, recovery from illness and stress. See e.g., U.S. Pat. No. 9,730,911.

β-Caryophyllene—naturally occurring in black pepper, rosemary, oregano, and cloves. It is the only terpene known to directly interact with CB2 receptors. It has illustrated antibacterial and antioxidant properties (see e.g., Dahham, S S., Molecules. 20(7):11808-11829 (2015)), as well as promising results in pain management studies (see e.g., Katsuyama, S., European Journal of Pain. 17(5):664-675 (2013)). In high amounts, it is a calcium and potassium ion channel blocker. As a result, it can impede the pressure exerted by heart muscles.

Terpinolene—present in many cannabis strains, but usually only in small quantities. This terpene has a multi-dimensional aroma that smells like pine trees, citrus, herbs, and florals. It has illustrated antioxidant property (see e.g., Turkez, H., Cytotechnology., 67(3):409-418 (2015)) and sedative property (see e.g., Ito, K. et al., Journal of Natural Medicines., 67(4):833-837 (2013)). Terpinolene is naturally occurring in nutmeg, tea tree, apples, and conifers.

Cineole/Eucalyptol—has spicy, camphor, refreshing, and minty scent. It can be used to increase circulation and pain relief. It easily crosses the blood-brain-barrier to trigger fast olfactory reaction. See e.g., U.S. Pat. No. 9,730,911.

Pulegone—has aroma that smells like mint, camphor, rosemary, and candy. Pulegone is an acetylcholinesterase inhibitor. That is, it stops the action of the protein that destroys acetylcholine, which is used by the brain to store memories. See e.g., U.S. Pat. No. 9,730,911.

The union between network pharmacology and traditional herbal pharmacotherapy, which is known for combining plant species and/or nutraceuticals, can result in targeted mixtures in order to ameliorate pathophysiological processes.

The partial perturbations of medicinal plants on a pharmacological network mimic physiological scenarios where hundreds of different enzyme systems and receptor types and subtypes act in concert. See e.g., Ágoston V. et al., Phys Rev E., 71(5):1-8 (2005). Some studies of cellular networks demonstrate that pharmacological agents that provide multiple signals can impact the complex equilibrium of whole cellular networks more favorably than drugs that act on a single target. See e.g., Csermely P. et al., Trends Pharmacol Sci., 26(4):178-182 (2005); Keith, C. T. & Zimmermann, G. R., Multi-target lead discovery for networked systems. Current Drug Discovery. 19-23 (2004); Morphy R. et al., Drug Discov Today, 9(15):641 (2004); Csermely P., Trends Biochem Sci., 29(7):331-334 (2004); Keith C T. et al., Nat Rev Drug Discov., 4(1):71-78 (2005); Briskin D P., Plant Physiol., 124(2):507-514 (2000); Werner E., Drug Discov Today, 8(24):1121-1127 (2003); Agoston V. et al., Phys Rev E., 71(5):1-8 (2005).

Additionally, multi-target agents may affect their targets only partially, which corresponds well with the presumed low-affinity interactions of medicinal plants. See e.g., Agoston V. et al., Phys Rev E., 71(5):1-8 (2005). Broader specificity, lower affinity, multi-component compounds have been reported to be, in some cases, more efficient than high affinity, high specificity compounds. See e.g., Csermely P., Trends Biochem Sci., 29(7):331-334 (2004); Lipton S A., Nature., 428(6982):473 (2004); Vickers A. et al., BMJ., 319(7216):1050-1053 (1999). Other advantages of low-affinity, multi-target pharmacological strategies include improved safety profiles (lower occurrence and reduced range of side effects) than high affinity, single-target drugs. A further advantage of traditional herbal formulas is the reduction of adverse events by buffering toxicity, (e.g., of THC), that an herb or specific constituent can contain. See e.g., Morphy R., et al., Drug Discov Today, 9(15):641 (2004); Ideker T. et al., Trends Biotechnol., 21(6):255 (2003); Vickers A. et al., BMJ., 319(7216):1050-1053 (1999); Jia W. et al., Phytother Res., 18(8):681-686 (2004); Shu H H. et al., J Ethnopharmacol. 103(3):398-405 (2006); Hikino H. et al., J Pharmacobiodyn., 3(10):514-525 (1980); Suzuki Y. et al., Planta Medica., 60(5):391-394 (1994).

Described herein are the compositions comprising at least one negative allosteric modulator of the cannabinoid receptor 1 (CB1), at least one partial agonist of CB1, and at least one gamma-aminobutyric acid (GABA)A receptor (GABAAR) activator and/or at least one transient receptor potential cation channel, subfamily A, member 1 (TRPA1) activator. In certain aspects, at least one negative allosteric modulator of CB1 is CBD and at least one partial agonist of CB1 is CBN. In certain aspects, CBD and CBN are present in a predefined ratio by weight. In some aspects, CBD:CBN ratio is about 2, about 3, about 4, about 5, about 6, about 7, about 8, about 9, about 10, about 11, about 12, about 13, about 14, about 15, about 16, about 17, about 18, about 19, or about 20.

### Compositions for Sleep Conditions

In certain aspects, the compositions of the present disclosure can be used for improving sleep quality.

In certain aspects, the compositions of the present disclosure comprise:
(i) about 5 mg to about 80 mg of cannabidiol (CBD), about 1 mg to about 15 mg of cannabinol (CBN), about 1 mg to about 30 mg of cannabigerol (CBG), or any combinations thereof;
(ii) one or more of the following: about 40 mg to 260 mg of Ziziphus spinosa, about 0.1 mg to 4 mg of melatonin, and about 30 mg to about 200 mg of Passiflora incarnata; and
(iii) three or more of the following: about 30 mg to about 150 mg eicosapentaenoic acid (EPA)/docosahexaenoic acid (DHA), about 1 mg to about 80 mg of Citrus reticulata (e.g., Citrus reticulata Blanco essential oil), about 50 mg to about 200 mg of Withania somnifera, about 30 mg to about 250 mg of Matricaria recutita, about 30 mg to about 220 mg of Lycopus spp., about 50 mg to about 200 mg of Poria cocos, about 5 mg to about 40 mg of Actaea racemosa, about 10 mg to about 150 mg of Pueraria lobata, about 15 mg to about 70 mg of Anemarrhena asphodeloides, about 30 mg to about 120 mg of phosphatidylserine, about 15 mg to about 70 mg of alpha lipoic acid, about 40 mg to about 150 mg of silymarin, about 100 mg to about 280 mg of Schisandra chinensis, about 5 mg to about 50 mg of Cymbopogon flexuosus essential oil, about 10 mg to about 120 mg of Humulus lupulus, about 50 mg to about 200 mg of Grindelia spp., about 30 mg to about 150 mg of Ligusticum wallichii, about 5 mg to about 50 mg of 5-hydroxytryptophan (5-HTP), about 15 mg to about 70 mg of paeonol, about 15 mg to about 150 mg of Panax ginseng, about 20 mg to about 100 mg of Rehmannia glutinosa, about 50 mg to about 200 mg of Polygala tenuifolia, about 20 mg to about 100 mg of Lavendula officinalis, about 50 mg to about 200 mg of Scutellaria lateriflora, about 50 mg to about 200 mg of Polygala tenuifolia, about 10 mg to about 150 mg of Pueraria lobata, about 5 mg to about 70 mg of Sceletium tortuosum, about 1 mg to about 15 mg of mixed Tocopherols, about 10 mg to about 200 mg of Magnolia officinalis, about 20 mg to about 100 mg of Piper methysticum, and about 1 mg to about 10 mg of Piper nigrum.

In certain aspects, the compositions of the present disclosure comprise:
(i) about 5 mg to about 80 mg of cannabidiol (CBD), about 1 mg to about 15 mg of cannabinol (CBN), about 1 mg to about 30 mg of cannabigerol (CBG), or any combinations thereof, and about 40 mg to 260 mg of Ziziphus spinosa; and
(ii) four or more of the following: about 0.1 mg to 4 mg of melatonin, about 30 mg to about 200 mg of Passiflora incarnata, about 30 mg to about 150 mg of eicosapentaenoic acid (EPA)/docosahexaenoic acid (DHA), about 1 mg to about 80 mg of Citrus reticulata (e.g., Citrus reticulata Blanco essential oil), about 50 mg to about 200 mg of Withania somnifera, about 30 mg to about 250 mg of Matricaria recutita, about 30 mg to about 220 mg of Lycopus spp., about 50 mg to about 200 mg of Poria cocos, about 5 mg to about 40 mg of Actaea racemosa, about 10 mg to about 150 mg of Pueraria lobata, about 15 mg to about 70 mg of Anemarrhena asphodeloides, about 30 mg to about 120 mg of phosphatidylserine, about 15 mg to about 70 mg of alpha lipoic acid, about 40 mg to about 150 mg of silymarin, about 100 mg to about 280 mg of Schisandra chinensis, about 5 mg to about 50 mg of Cymbopogon flexuosus essential oil, about 10 mg to about 120 mg of Humulus lupulus, about 50 mg to about 200 mg of Grindelia spp., about 30 mg to about 150 mg of Ligusticum wallichii, about 5 mg to about 50 mg of 5-hydroxytryptophan (5-HTP), about 15 mg to about 70 mg of paeonol, about 15 mg to about 150 mg of Panax ginseng, about 20 mg to about 100 mg of Rehmannia glutinosa, about 50 mg to about 200 mg of Polygala tenuifolia, about 20 mg to about 100 mg of Lavendula officinalis, about 50 mg to about 200 mg of Scutellaria lateriflora, about 50 mg to about 200 mg of Polygala tenuifolia, about 10 mg to about 150 mg of Pueraria lobata, about 5 mg to about 70 mg of Sceletium tortuosum, about 1 mg to about 15 mg of mixed Tocopherols, about 10 mg to about 200 mg of Magnolia officinalis, about 20 mg to about 100 mg of Piper methysticum, and about 1 mg to about 10 mg of Piper nigrum.

In some aspects, the compositions of the present disclosure comprise:
(i) about 5 mg to about 80 mg of cannabidiol (CBD), about 1 mg to about 15 mg of cannabinol (CBN), about 1 mg to about 30 mg of cannabigerol (CBG), or any combinations thereof, and about 0.1 mg to 4 mg of melatonin; and
(ii) four or more of the following: about 40 mg to 260 mg of Ziziphus spinosa, about 30 mg to about 200 mg of Passiflora incarnata; about 30 mg to about 150 mg of eicosapentaenoic acid (EPA)/docosahexaenoic acid (DHA), about 1 mg to about 80 mg of Citrus reticulata (e.g., Citrus reticulata Blanco essential oil), about 50 mg to about 200 mg of Withania somnifera, about 30 mg to about 250 mg of Matricaria recutita, about 30 mg to about 220 mg of Lycopus spp., about 50 mg to about 200 mg of Poria cocos, about 5 mg to about 40 mg of Actaea racemosa, about 10 mg to about 150 mg of Pueraria lobata, about 15 mg to about 70 mg of Anemarrhena asphodeloides, about 30 mg to about 120 mg of phosphatidylserine, about 15 mg to about 70 mg of alpha lipoic acid, about 40 mg to about 150 mg of silymarin, about 100 mg to about 280 mg of Schisandra chinensis, about 5 mg to about 50 mg of Cymbopogon flexuosus essential oil, about 10 mg to about 120 mg of Humulus lupulus, about 50 mg to about 200 mg of Grindelia spp., about 30 mg to about 150 mg of Ligusticum wallichii, about 5 mg to about 50 mg of 5-hydroxytryptophan (5-HTP), about 15 mg to about 70 mg of paeonol, about 15 mg to about 150 mg of Panax ginseng, about 20 mg to about 100 mg of Rehmannia glutinosa, about 50 mg to about 200 mg of Polygala tenuifolia, about 20 mg to about 100 mg of Lavendula officinalis, about 50 mg to about 200 mg of Scutellaria lateriflora, about 50 mg to about 200 mg of Polygala tenuifolia, about 10 mg to about 150 mg of Pueraria lobata, about 5 mg to about 70 mg of Sceletium tortuosum, about 1 mg to about 15 mg of mixed Tocopherols, about 10 mg to about 200 mg of Magnolia officinalis, about 20 mg to about 100 mg of Piper methysticum, and about 1 mg to about 10 mg of Piper nigrum.

In certain aspects, the compositions of the present disclosure comprise:
(i) about 5 mg to about 80 mg of cannabidiol (CBD), about 1 mg to about 15 mg of cannabinol (CBN), about 1 mg to about 30 mg of cannabigerol (CBG), or any combinations thereof, and about 30 mg to about 200 mg of Passiflora incarnata; and
(ii) four or more of the following: about 0.1 mg to 4 mg of melatonin; about 40 mg to 260 mg of Ziziphus spinosa, about 30 mg to about 150 mg of eicosapentaenoic acid (EPA)/docosahexaenoic acid (DHA), about 1 mg to about 80 mg of Citrus reticulata (e.g., Citrus reticulata Blanco essential oil), about 50 mg to about 200 mg of Withania somnifera, about 30 mg to about 250 mg of Matricaria recutita, about 30 mg to about 220 mg of Lycopus spp., about 50 mg to about 200 mg of Poria cocos, about 5 mg to about 40 mg of Actaea racemosa, about 10 mg to about 150 mg of Pueraria lobata, about 15 mg to about 70 mg of Anemarrhena asphodeloides, about 30 mg to about 120 mg of phosphatidylserine, about 15 mg to about 70 mg of alpha lipoic acid, about 40 mg to about 150 mg of silymarin, about 100 mg to about 280 mg of Schisandra chinensis, about 5 mg to about 50 mg of Cymbopogon flexuosus essential oil, about 10 mg to about 120 mg of Humulus lupulus, about 50 mg to about 200 mg of Grindelia spp., about 30 mg to about 150 mg of Ligusticum wallichii, about 5 mg to about 50 mg of 5-hydroxytryptophan (5-HTP), about 15 mg to about 70 mg of paeonol, about 15 mg to about 150 mg of Panax ginseng, about 20 mg to about 100 mg of Rehmannia glutinosa, about 50 mg to about 200 mg of Polygala tenuifolia, about 20 mg to about 100 mg of Lavendula officinalis, about 50 mg to about 200 mg of Scutellaria lateriflora, about 50 mg to about 200 mg of Polygala tenuifolia, about 10 mg to about 150 mg of Pueraria lobata, about 5 mg to about 70 mg of Sceletium tortuosum, about 1 mg to about 15 mg of mixed Tocopherols, about 10 mg to about 200 mg of Magnolia officinalis, about 20 mg to about 100 mg of Piper methysticum, and about 1 mg to about 10 mg of Piper nigrum.

In certain aspects, the compositions of the present disclosure comprise CBD in the amount of about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 68 mg, about 59 mg, about 60 mg, about 61 mg, about 62 mg, about 63 mg, about 64 mg, about 65 mg, about 66 mg, about 67 mg, about 68 mg, about 69, mg, about 70 mg, about 71 mg, about 72 mg, about 73 mg, about 74 mg, about 75 mg, about 76 mg, about 77 mg, about 78 mg, about 79 mg, or about 80 mg. In certain aspects, the compositions of the present disclosure comprise about 10 mg of CBD. In certain aspects, the compositions of the present disclosure comprise 10 mg of CBD. In certain aspects, the compositions of the present disclosure comprise about 15 mg of CBD. In certain aspects, the compositions of the present disclosure comprise 15 mg of CBD. In certain aspects, the compositions of the present disclosure comprise about 20 mg of CBD. In certain aspects, the compositions of the present disclosure comprise 20 mg of CBD. In certain aspects, the compositions of the present disclosure comprise about 30 mg of CBD. In certain aspects, the compositions of the present disclosure comprise 30 mg of CBD. In certain aspects, the compositions of the present disclosure comprise about 60 mg of CBD. In certain aspects, the compositions of the present disclosure comprise 60 mg of CBD.

In certain aspects, the compositions of the present disclosure comprise CBN in the amount of about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.1 mg, about 2.2 mg, about 2.3 mg, about 2.4 mg, about 2.5 mg, about 2.6 mg, about 2.7 mg, about 2.8 mg, about 2.9 mg, about 3 mg, about 3.1 mg, about 3.2 mg, about 3.3 mg, about 3.4 mg, about 3.5 mg, about 3.6 mg, about 3.7 mg, about 3.8 mg, about 3.9 mg, about 4 mg, about 4.1 mg, about 4.2 mg, about 4.3 mg, about 4.4 mg, about 4.5 mg, about 4.6 mg, about 4.7 mg, about 4.8 mg, about 4.9 mg, about 5 mg, about 5.1 mg, about 5.2 mg, about 5.3 mg, about 5.4 mg, about 5.5 mg, about 5.6 mg, about 5.7 mg, about 5.8 mg, about 5.9 mg, about 6 mg, about 6.1 mg, about 6.2 mg, about 6.3 mg, about 6.4 mg, about 6.5 mg, about 6.6 mg, about 6.7 mg, about 6.8 mg, about 6.9 mg, about 7 mg, about 7.1 mg, about 7.2 mg, about 7.3 mg, about 7.4 mg, about 7.5 mg, about 7.6 mg, about 7.7 mg, about 7.8 mg, about 7.9 mg, about 8 mg, about 8.1 mg, about 8.2 mg, about 8.3 mg, about 8.4 mg, about 8.5 mg, about 8.6 mg, about 8.7 mg, about 8.8 mg, about 8.9 mg, about 9 mg, about 9.1 mg, about 9.2 mg, about 9.3 mg, about 9.4 mg, about 9.5 mg, about 9.6 mg, about 9.7 mg, about 9.8 mg, about 9.9 mg, about 10 mg, about 10.1 mg, about 10.2 mg, about 10.3 mg, about 10.4 mg, about 10.5 mg, about 10.6 mg, about 10.7 mg, about 10.8 mg, about 10.9 mg, about 11 mg, about 11.1 mg, about 11.2 mg, about 11.3 mg, about 11.4 mg, about 11.5 mg, about 11.6 mg, about 11.7 mg, about 11.8 mg, about 11.9 mg, about 12 mg, about 12.1 mg, about 12.2 mg, about 12.3 mg, about 12.4 mg, about 12.5 mg, about 12.6 mg, about 12.7 mg, about 12.8 mg, about 12.9 mg, about 13 mg, about 13.1 mg, about 13.2 mg, about 13.3 mg, about 13.4 mg, about 13.5 mg, about 13.6 mg, about 13.7 mg, about 13.8 mg, about 13.9 mg, about 14 mg, about 14.1 mg, about 14.2 mg, about 14.3 mg, about 14.4 mg, about 14.5 mg, about 14.6 mg, about 14.7 mg, about 14.8 mg, about 14.9 mg, about 15 mg, about 20 mg, about 25 mg, or about 30 mg. In certain aspects, the compositions of the present disclosure comprise about 2.5 mg of CBN. In certain aspects, the compositions of the present disclosure comprise 2.5 mg of CBN. In certain aspects, the compositions of the present disclosure comprise about 5 mg of CBN. In certain aspects, the compositions of the present disclosure comprise 5 mg of CBN. In certain aspects, the compositions of the present disclosure comprise about 10 mg of CBN. In certain aspects, the compositions of the present disclosure comprise 10 mg of CBN.

In certain aspects, the compositions of the present disclosure comprise CBG in the amount of about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.1 mg, about 2.2 mg, about 2.3 mg, about 2.4 mg, about 2.5 mg, about 2.6 mg, about 2.7 mg, about 2.8 mg, about 2.9 mg, about 3 mg, about 3.1 mg, about 3.2 mg, about 3.3 mg, about 3.4 mg, about 3.5 mg, about 3.6 mg, about 3.7 mg, about 3.8 mg, about 3.9 mg, about 4 mg, about 4.1 mg, about 4.2 mg, about 4.3 mg, about 4.4 mg, about 4.5 mg, about 4.6 mg, about 4.7 mg, about 4.8 mg, about 4.9 mg, about 5 mg, about 5.1 mg, about 5.2 mg, about 5.3 mg, about 5.4 mg, about 5.5 mg, about 5.6 mg, about 5.7 mg, about 5.8 mg, about 5.9 mg, about 6 mg, about 6.1 mg, about 6.2 mg, about 6.3 mg, about 6.4 mg, about 6.5 mg, about 6.6 mg, about 6.7 mg, about 6.8 mg, about 6.9 mg, about 7 mg, about 7.1 mg, about 7.2 mg, about 7.3 mg, about 7.4 mg, about 7.5 mg, about 7.6 mg, about 7.7 mg, about 7.8 mg, about 7.9 mg, about 8 mg, about 8.1 mg, about 8.2 mg, about 8.3 mg, about 8.4 mg, about 8.5 mg, about 8.6 mg, about 8.7 mg, about 8.8 mg, about 8.9 mg, about 9 mg, about 9.1 mg, about 9.2 mg, about 9.3 mg, about 9.4 mg, about 9.5 mg, about 9.6 mg, about 9.7 mg, about 9.8 mg, about 9.9 mg, about 10 mg, about 10.1 mg, about 10.2 mg, about 10.3 mg, about 10.4 mg, about 10.5 mg, about 10.6 mg, about 10.7 mg, about 10.8 mg, about 10.9 mg, about 11 mg, about 11.1 mg, about 11.2 mg, about 11.3 mg, about 11.4 mg, about 11.5 mg, about 11.6 mg, about 11.7 mg, about 11.8 mg, about 11.9 mg, about 12 mg, about 12.1 mg, about 12.2 mg, about 12.3 mg, about 12.4 mg, about 12.5 mg, about 12.6 mg, about 12.7 mg, about 12.8 mg, about 12.9 mg, about 13 mg, about 13.1 mg, about 13.2 mg, about 13.3 mg, about 13.4 mg, about 13.5 mg, about 13.6 mg, about 13.7 mg, about 13.8 mg, about 13.9 mg, about 14 mg, about 14.1 mg, about 14.2 mg, about 14.3 mg, about 14.4 mg, about 14.5 mg, about 14.6 mg, about 14.7 mg, about 14.8 mg, about 14.9 mg, about 15 mg, about 20 mg, about 25 mg, or about 30 mg. In certain aspects, the compositions of the present disclosure comprise about 2.5 mg of CBG. In certain aspects, the compositions of the present disclosure comprise 2.5 mg of CBG. In certain aspects, the compositions of the present disclosure comprise about 5 mg of CBG. In certain aspects, the compositions of the present disclosure comprise 5 mg of CBG. In certain aspects, the compositions of the present disclosure comprise about 10 mg of CBG. In certain aspects, the compositions of the present disclosure comprise 10 mg of CBG.

In certain aspects, the compositions of the present disclosure comprise Ziziphus spinosa in the amount of about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg. In certain aspects, the compositions of the present disclosure comprise about 50 mg of Ziziphus spinosa. In certain aspects, the compositions of the present disclosure comprise 50 mg of Ziziphus spinosa. In certain aspects, the compositions of the present disclosure comprise about 75 mg of Ziziphus spinosa. In certain aspects, the compositions of the present disclosure comprise 75 mg of Ziziphus spinosa. In certain aspects, the compositions of the present disclosure comprise about 100 mg of Ziziphus spinosa. In certain aspects, the compositions of the present disclosure comprise 100 mg of Ziziphus spinosa. In certain aspects, the compositions of the present disclosure comprise about 150 mg of Ziziphus spinosa. In certain aspects, the compositions of the present disclosure comprise 150 mg of Ziziphus spinosa. In certain aspects, the compositions of the present disclosure comprise about 200 mg of Ziziphus spinosa. In certain aspects, the compositions of the present disclosure comprise 200 mg of Ziziphus spinosa.

Ziziphus is a genus of about 40 species of spiny shrubs and small trees in the buckthorn family, Rhamnaceae, distributed in the warm-temperate and subtropical regions throughout the world. See e.g., Guo et al., Front. Pharmacol., 8:609 (2017).

Ziziphus species of the present disclosure include, but are not limited to, Ziziphus spinosa (Ziziphus jujuba or jujube), Ziziphus spina-christi, Ziziphus lotus, Ziziphus mauritiana, Ziziphus joazeiro, and Ziziphus celata.

Ziziphus spinosa can activate gamma-aminobutyric acid (GABA)A receptor, upregulate Nuclear factor erythroid 2-related factor 2 (Nrf2) nuclear translocation, prevent N-methyl-D-aspartate (NMDA)-induced neuronal cell damage, increase synaptic activity via α-amino-3-hydroxy-5-methyl-4-isoxazolepropionic acid (AMPA), increase synaptic AMPA receptor (AMPAR) transmission via mitogen-activated protein kinase (MAPK), adenylyl cyclase (AC), and p21-activated protein kinase (PAK), inhibit glutamate-mediated pathways in the hippocampus, enhance expression of glutamic acid decarboxylase, and/or decrease expression of NMDAR1. See e.g., Ma Y et al, Biol Pharm Bull. 31(9):1749-1754 (2008); Park J H et al., J Ethnopharmacol., 95(1):39-45 (2004); Jo S Y. et al., J Ethnopharmacol., 200:16-21 (2017); Zhang M. et al., Planta Med. 69(8):692-695 (2003); Han H. et al., Pharmacol Biochem Behav., 92(2):206-213 (2009).

In certain aspects, the compositions of the present disclosure comprise at least one gamma-aminobutyric acid (GABA)A receptor activator. In certain aspects, at least one gamma-aminobutyric acid (GABA)A receptor activator is Ziziphus spinosa.

In certain aspects, the compositions of the present disclosure comprise melatonin in the amount of about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.1 mg, about 2.2 mg, about 2.3 mg, about 2.4 mg, about 2.5 mg, about 2.6 mg, about 2.7 mg, about 2.8 mg, about 2.9 mg, about 3 mg, about 3.1 mg, about 3.2 mg, about 3.3 mg, about 3.4 mg, about 3.5 mg, about 3.6 mg, about 3.7 mg, about 3.8 mg, about 3.9 mg, or about 4 mg. In certain aspects, the compositions of the present disclosure comprise about 0.5 mg of melatonin. In certain aspects, the compositions of the present disclosure comprise 0.5 mg of melatonin. In certain aspects, the compositions of the present disclosure comprise about 1.25 mg of melatonin. In certain aspects, the compositions of the present disclosure comprise 1.25 mg of melatonin. In certain aspects, the compositions of the present disclosure comprise about 1.5 mg of melatonin. In certain aspects, the compositions of the present disclosure comprise 1.5 mg of melatonin. In certain aspects, the compositions of the present disclosure comprise about 2.5 mg of melatonin. In certain aspects, the compositions of the present disclosure comprise 2.5 mg of melatonin. In certain aspects, the compositions of the present disclosure comprise about 3 mg of melatonin. In certain aspects, the compositions of the present disclosure comprise 3 mg of melatonin.

In certain aspects, the compositions of the present disclosure comprise lower doses of indigents (e.g., melatonin) that target multiple targets on the endocannabinoid system (ECS) and other enzymes and receptors that help control sleep, pain, and anxiety. In certain aspects, a lower dose of an ingredient in the compositions described herein (e.g., concentration of melatonin between about 0.15% to about 0.6%), can cause lower occurrence and reduced range of side effects (or no side effects) compared to a higher dose of an ingredient (e.g., concentration of melatonin of about 3% of melatonin) that can cause addiction and/or reduction of the human body's ability to heal itself.

Melatonin or 5 methoxy-N-acetyltryptamine is a hormone found naturally in the body. Melatonin regulates circadian rhythms such as the sleep-wake rhythm, neuroendocrine rhythms or body temperature cycles through its action on MT1 and MT2 receptors, and sleep disorders (such as increased sleep latency, reduced total sleep, and nocturnal awakenings with insomnia). Ingestion of melatonin induces fatigue, sleepiness and a diminution of sleep latency. Disturbed circadian rhythms are associated with sleep disorders and impaired health. See e.g., Tordjman S. et al., Curr Neuropharmacol., 15(3):434-443 (2017). In addition, melatonin is used for improving sleep in difficult sleep conditions such as jet lag, for adjusting sleep-wake cycles in people whose daily work schedule changes (shift-work disorder), and for helping people establish a day and night cycle. Melatonin can activate (GABA)A receptor, inhibit dopamine transporter and glutamate receptor, and/or increase acetylcholine secretion in the hippocampus. See e.g., Sato S. et al., J Physiol Sci., 58(5):317-322 (2008); Zisapel N. et al., Cell Mol Neurobiol., 21(6):605-616 (2001); Meng T. et al., Neuroscience., 286:308-315 (2015).

In certain aspects, the compositions of the present disclosure comprise at least one gamma-aminobutyric acid (GABA)A receptor activator. In certain aspects, at least one gamma-aminobutyric acid (GABA)A receptor activator is melatonin.

In certain aspects, the compositions of the present disclosure comprise Passiflora incarnata in the amount of about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg. In certain aspects, the compositions of the present disclosure comprise about 50 mg of Passiflora incarnata. In certain aspects, the compositions of the present disclosure comprise 50 mg of Passiflora incarnata. In certain aspects, the compositions of the present disclosure comprise about 90 mg of Passiflora incarnata. In certain aspects, the compositions of the present disclosure comprise 90 mg of Passiflora incarnata. In certain aspects, the compositions of the present disclosure comprise about 100 mg of Passiflora incarnata. In certain aspects, the compositions of the present disclosure comprise 100 mg of Passiflora incarnata. In certain aspects, the compositions of the present disclosure comprise about 180 mg of Passiflora incarnata. In certain aspects, the compositions of the present disclosure comprise 180 mg of Passiflora incarnata.

Passiflora, known also as the passion flowers or passion vines, is a genus of about 550 species of flowering plants, the type genus of the family Passifloraceae. See e.g., Cerqueira-Silva C B. et al., Int J Mol Sci., 15(8):14122-14152 (2014).

Passiflora species of the present disclosure include, but are not limited to, Passiflora incarnata (maypops—passion flower, purple passionflower, apricot vine, maypop, wild passion flower, see e.g., Park J W et al., Int J Mol Med., 41(6):3709-3716 (2018)), Passiflora actinia (passion flower), Passiflora antioquiensis (banana passionfruit), Passiflora caerulea (passion flower, bluecrown passionflower, blue passion flower), Passiflora edulis (passion flower, purple granadilla), Passiflora herbertiana, Passiflora laurifolia (yellow granadilla), Passiflora ligularis (sweet grenadilla, passion flower), Passiflora membranacea (passion flower), Passiflora mollisima (banana passion fruit), Passiflora popenovii (granadilla de quijos), Passiflora quadrangularis (giant granadilla, badea), Passiflora tarminiana (banana passionfruit, banana poka), Passiflora tetrandra, Passiflora tripartita mollissima (banana passionfruit), Passiflora umbilicata (passion flower), Passiflora×colvillii (passion flower), and Passiflora×exoniensis (passion flower).

Passiflora incarnata can activate (GABA)A receptor and 5-hydroxytryptamine (5-HT) receptor, modulate the opioid neuropeptide systems, enhance activity of extracellular-signal-regulated kinase (ERK)1/2, inhibit COX-2 expression, and/or inhibit activation of NFkB. In certain aspects, the compositions of the present disclosure comprise at least one gamma-aminobutyric acid (GABA)A receptor activator. In certain aspects, at least one gamma-aminobutyric acid (GABA)A receptor activator is Passiflora incarnata. See e.g., Appel K. et al., Phytother Res., 25(6):838-843 (2011); Park J W, et al., Int J Molec Med, 41(6):3709-3716 (2018).

In certain aspects, the compositions of the present disclosure comprise eicosapentaenoic acid (EPA)/docosahexaenoic acid (DHA) in the amount of about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 120 mg, about 130 mg, about 135 mg, about 140 mg, about 145 mg, about 150 mg, about 155 mg, about 160 mg, about 165 mg, about 170 mg, about 175 mg, or about 180 mg. In certain aspects, the compositions of the present disclosure comprise about 50 mg of EPA/DHA. In certain aspects, the compositions of the present disclosure comprise 50 mg of EPA/DHA. In certain aspects, the compositions of the present disclosure comprise about 100 mg of EPA/DHA. In certain aspects, the compositions of the present disclosure comprise 100 mg of EPA/DHA.

In certain aspects, the compositions of the present disclosure comprise one or more essential oils from Citrus species. In some aspects, the Citrus species is Citrus reticulata (e.g., Citrus reticulate Blanco essential oil). In certain aspects, the compositions of the present disclosure comprise Citrus reticulata (e.g., Citrus reticulata Blanco essential oil) in the amount of about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, 30 mg, about 35 mg, about 40 mg, about 45 mg, about 50 mg, about 55 mg, about 60 mg, about 65 mg, about 70 mg, about 75 mg, or about 80 mg. In certain aspects, the compositions of the present disclosure comprise about 25 mg of Citrus reticulata (e.g., Citrus reticulata Blanco essential oil). In certain aspects, the compositions of the present disclosure comprise 25 mg of Citrus reticulata (e.g., Citrus reticulata Blanco essential oil). In certain aspects, the compositions of the present disclosure comprise about 30 mg of Citrus reticulata (e.g., Citrus reticulata Blanco essential oil). In certain aspects, the compositions of the present disclosure comprise 30 mg of Citrus reticulata (e.g., Citrus reticulata Blanco essential oil). In certain aspects, the compositions of the present disclosure comprise about 50 mg of Citrus reticulata (e.g., Citrus reticulata Blanco essential oil). In certain aspects, the compositions of the present disclosure comprise 50 mg of Citrus reticulata (e.g., Citrus reticulata Blanco essential oil). In certain aspects, the compositions of the present disclosure comprise about 60 mg of Citrus reticulata (e.g., Citrus reticulata Blanco essential oil). In certain aspects, the compositions of the present disclosure comprise 60 mg of Citrus reticulata (e.g., Citrus reticulata Blanco essential oil).

In certain aspects, the compositions of the present disclosure comprise Withania somnifera in the amount of about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, 310 mg, about 315 mg, about 320 mg, about 325 mg, about 330 mg, about 335 mg, about 340 mg, about 345 mg, about 350 mg, about 355, about 360 mg, about 365 mg, about 370 mg, about 375 mg, about 380 mg, about 385 mg, about 390 mg, about 395 mg, about 400 mg, about 405 mg, 410 mg, about 415 mg, about 420 mg, about 425 mg, about 430 mg, about 435 mg, about 440 mg, about 445 mg, or about 450 mg. In certain aspects, the compositions of the present disclosure comprise about 75 mg of Withania somnifera. In certain aspects, the compositions of the present disclosure comprise 75 mg of Withania somnifera. In certain aspects, the compositions of the present disclosure comprise about 150 mg of Withania somnifera. In certain aspects, the compositions of the present disclosure comprise 150 mg of Withania somnifera.

Withania is a genus of flowering plants in the nightshade family, Solanaceae, with about 23 species that are native to parts of North Africa, western Asia, south Asia, southern Europe, the Mediterranean, and the Canary Islands. See e.g., Mirjalili, M. H. et al., Molecules., 14(7): 2373-2393 (2009); “Withania somnifera.” Germplasm Resources Information Network (GRIN). Agricultural Research Service (ARS), United States Department of Agriculture (USDA). Accessed 28 Apr. 2020.

Withania species of the present disclosure include, but are not limited to, Withania somnifera (ashwaganda, Indian ginseng, winter cherry), Withania adpressa, Withania adunensis, Withania begoniifolia, Withania chevalier, Withania coagulans, Withania frutescens, Withania japonica, Withania qaraitica, Withania reichenbachii, Withania riebeckii, and Withania sphaerocarpa.

In certain aspects, the compositions of the present disclosure comprise Matricaria recutita in the amount of about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, or about 250 mg. In certain aspects, the compositions of the present disclosure comprise about 50 mg of Matricaria recutita. In certain aspects, the compositions of the present disclosure comprise 50 mg of Matricaria recutita. In certain aspects, the compositions of the present disclosure comprise about 100 mg of Matricaria recutita. In certain aspects, the compositions of the present disclosure comprise 100 mg of Matricaria recutita. In certain aspects, the compositions of the present disclosure comprise about 200 mg of Matricaria recutita. In certain aspects, the compositions of the present disclosure comprise 200 mg of Matricaria recutita.

Matricaria is a widespread genus of flowering plants of the family Asteraceae that grow in temperate regions of Europe, Asia, America and Africa. See e.g., Sharifi-Rada M. et al., Microbial Research, 215:76-88 (2018). Some of the species are also naturalized in Australia. Matricaria species of the present disclosure include, but are not limited to, Matricaria recutita (also knows as Matricaria chamomilla, chamomile, German chamomile, Hungarian chamomile (kamilla), wild chamomile, blue chamomile, scented mayweed), Matricaria aserbaidshanica, Matricaria aurea, Matricaria australis, Matricaria brachyglossa, Matricaria breviradiata, Matricaria chamomilla, Matricaria courrantiana, Matricaria decipiens, Matricaria discoidea, Matricaria elongate, Matricaria grossheimii, Matricaria karjaginii, Matricaria lasiocarpa, Matricaria matricarioides, Matricaria occidentalis, Matricaria rupestris, Matricaria sevanensis, Matricaria subpolaris, Matricaria szowitzii, Matricaria tetragonosperma, and Matricaria transcaucasica.

In certain aspects, the compositions of the present disclosure comprise Lycopus spp. in the amount of about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, or about 220 mg. In certain aspects, the compositions of the present disclosure comprise about 50 mg of Lycopus spp. In certain aspects, the compositions of the present disclosure comprise 50 mg of Lycopus spp. In certain aspects, the compositions of the present disclosure comprise about 100 mg of Lycopus spp. In certain aspects, the compositions of the present disclosure comprise 100 mg of Lycopus spp. In certain aspects, the compositions of the present disclosure comprise about 200 mg of Lycopus spp. In certain aspects, the compositions of the present disclosure comprise 200 mg of Lycopus spp.

Lycopus (water horehound, gypsywort, or bugleweed) is a genus in the family Lamiaceae. They are all herbaceous plants native to Europe, Asia, Australia, and North America. See e.g., Trajcikovi E. et al., Molecules., 25(6):1422 (published online March 2020). Lycopus species of the present disclosure include, but are not limited to, Lycopus alissoviae, Lycopus americanus, Lycopus amplectens, Lycopus angustifolius, Lycopus asper, Lycopus australis, Lycopus cavaleriei, Lycopus charkeviczii, Lycopus cokeri, Lycopus europaeus, Lycopus exaltatus, Lycopus hirtellus, Lycopus×intermedius, Lycopus kurilensis, Lycopus laurentianus, Lycopus lucidus, Lycopi rhizome, Lycopus rubellus, Lycopus×sherardii, Lycopus sichotensis, Lycopus uniflorus, and Lycopus virginicus.

In certain aspects, the compositions of the present disclosure comprise Poria cocos in the amount of about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, or about 300 mg. In certain aspects, the compositions of the present disclosure comprise about 75 mg of Poria cocos. In certain aspects, the compositions of the present disclosure comprise 75 mg of Poria cocos. In certain aspects, the compositions of the present disclosure comprise about 150 mg of Poria cocos. In certain aspects, the compositions of the present disclosure comprise 150 mg of Poria cocos.

Wolfiporia is a genus of fungi in the family Polyporaceae. See e.g., Wei W. et al., Front Microbiol., 7:1495 (2016). Wolfiporia species of the present disclosure include, but are not limited to, Poria cocos (Wolfiporia extensa), Wolfiporia cartilaginea, Wolfiporia castanopsis, Wolfiporia curvispora, Wolfiporia dilatohypha, and Wolfiporia sulphurea.

In certain aspects, the compositions of the present disclosure comprise Actaea racemosa in the amount of about 5 mg, about 10 mg, about 15 mg, about 20 mg, about 25 mg, about 30 mg, about 35 mg, or about 40 mg. In certain aspects, the compositions of the present disclosure comprise about 15 mg of Actaea racemosa. In certain aspects, the compositions of the present disclosure comprise 15 mg of Actaea racemosa. In certain aspects, the compositions of the present disclosure comprise about 30 mg of Actaea racemosa. In certain aspects, the compositions of the present disclosure comprise 30 mg of Actaea racemosa.

Actaea (commonly called baneberry, bugbane and cohosh), is a genus of flowering plants of the family Ranunculaceae, native to subtropical, temperate and subarctic regions of Europe, Asia and North America. See e.g., Rhuy M-R. et al., J Agric Food Chem., 27; 54(26):9852-9857 (2007). Actaea species of the present disclosure include, but are not limited to, Actaea racemosa (black cohosh, black bugbane), Actaea arizonica, Arizona bugbane, Actaea asiatica, Actaea elata, Actaea matsumurae (Kamchatka bugbane, Japanese bugbane), Actaea pachypoda (white baneberry, white cohosh, doll's eyes), Actaea podocarpa, Actaea rubra (Actaea erythrocarpa, red baneberry), Actaea simplex, and Actaea spicata (Actaea alba, baneberry, herb christopher).

In certain aspects, the compositions of the present disclosure comprise Pueraria lobata in the amount of about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg. In certain aspects, the compositions of the present disclosure comprise about 25 mg of Pueraria lobata. In certain aspects, the compositions of the present disclosure comprise 25 mg of Pueraria lobata. In certain aspects, the compositions of the present disclosure comprise about 50 mg of Pueraria lobata. In certain aspects, the compositions of the present disclosure comprise 50 mg of Pueraria lobata. In certain aspects, the compositions of the present disclosure comprise about 100 mg of Pueraria lobata. In certain aspects, the compositions of the present disclosure comprise 100 mg of Pueraria lobata.

Pueraria is a plant genus of 15-20 species in family Fabaceae, native to Asia. See e.g., Wang Q., et al., American Journal of Botany, 97(12): 1982-1998 (2010). Pueraria species of the present disclosure include, but are not limited to, Pueraria lobata (kudzu, see e.g., Jung H W. et al., Nutrients., 9(1):33 (2017)), Pueraria edulis, Pueraria montana, Pueraria phaseoloides, Pueraria thomsonii, Pueraria bella, Pueraria mirifica, Pueraria omeiensis, Pueraria peduncularis, Pueraria tuberosa, and Pueraria wallichii.

In certain aspects, the compositions of the present disclosure comprise Anemarrhena asphodeloides in the amount of about 15 mg about 20 mg, about 25 mg, about 30 mg, about 35 mg, about 40 mg, about 45 mg, about 50 mg, about 55 mg, about 60 mg, about 65 mg, or about 70 mg. In certain aspects, the compositions of the present disclosure comprise about 25 mg of Anemarrhena asphodeloides. In certain aspects, the compositions of the present disclosure comprise 25 mg of Anemarrhena asphodeloides. In certain aspects, the compositions of the present disclosure comprise about 50 mg of Anemarrhena asphodeloides. In certain aspects, the compositions of the present disclosure comprise 50 mg of Anemarrhena asphodeloides.

Anemarrhena is a plant genus in family Asparagaceae, subfamily Agavoideae. It has only one species, Anemarrhena asphodeloides, native to China, Korea, and Mongolia. See e.g., Piwowar A., et al., Int. J Mol. Sci., 21(7):2510 (2020).

In certain aspects, the compositions of the present disclosure comprise phosphatidylserine in the amount of about 30 mg, about 40 mg, about 50 mg, about 60 mg, about 70 mg, about 80 mg, about 90 mg, about 100 mg, about 110 mg, or about 120 mg. In certain aspects, the compositions of the present disclosure comprise about 50 mg of phosphatidylserine. In certain aspects, the compositions of the present disclosure comprise 50 mg of phosphatidylserine. In certain aspects, the compositions of the present disclosure comprise about 100 mg of phosphatidylserine. In certain aspects, the compositions of the present disclosure comprise 100 mg of phosphatidylserine.

Phosphatidylserine (PS) is a naturally occurring phospholipid nutrient that is most concentrated in organs with high metabolic activity, such as the brain, lungs, heart, liver and skeletal muscle. PS is located mainly in the internal layer of the cell membrane and has a variety of unique regulatory and structural functions. It modulates the activity of receptors, enzymes, ion channels and signaling molecules and is involved in governing membrane fluidity. PS is considered to be an important brain nutrient beneficially influencing numerous neurotransmitter systems, such as acetylcholine, dopamine, serotonin and norepinephrine. See e.g., Hirayama S. et al., J Human Nutrition and Dietetics, 27(s2):284-291 (2014).

In certain aspects, the compositions of the present disclosure comprise alpha lipoic acid in the amount of about 15 mg, about 20 mg, about 25 mg, about 30 mg, about 35 mg, about 40 mg, about 45 mg, about 50 mg, about 55 mg, about 60 mg, about 65 mg, or about 70 mg. In certain aspects, the compositions of the present disclosure comprise about 25 mg of alpha lipoic acid. In certain aspects, the compositions of the present disclosure comprise 25 mg of alpha lipoic acid. In certain aspects, the compositions of the present disclosure comprise about 50 mg of alpha lipoic acid. In certain aspects, the compositions of the present disclosure comprise 50 mg of alpha lipoic acid.

α-lipoic acid (alpha lipoic acid, ALA, thioctic acid) is an organosulfur component produced from plants, animals, and humans. It has various properties, among them great antioxidant potential and is widely used as a racemic drug for diabetic polyneuropathy-associated pain and paresthesia. Naturally, ALA is located in mitochondria, where it is used as a cofactor for pyruvate dehydrogenase (PDH) and α-ketoglutarate dehydrogenase complexes. See e.g., Salehi B. et al., Biomolecules., 9(8):356 (2019).

In certain aspects, the compositions of the present disclosure comprise silymarin in the amount of about 40 mg, about 40 mg, about 60 mg, about 70 mg, about 80 mg, about 90 mg, about 100 mg, about 110 mg, about 120 mg, about 130 mg, about 140 mg, or about 150 mg. In certain aspects, the compositions of the present disclosure comprise about 60 mg of silymarin. In certain aspects, the compositions of the present disclosure comprise 60 mg of silymarin. In certain aspects, the compositions of the present disclosure comprise about 120 mg of silymarin. In certain aspects, the compositions of the present disclosure comprise 120 mg of silymarin.

Silymarin (Silybum marianum L., milk thistle) is a member of Carduus marianum family. It is an ancient medicinal plant which has been used for treatment of different diseases such as liver and gallbladder disorders, protecting liver against snake bite and insect stings, mushroom poisoning and alcohol abuse. Silymarin effects have also been indicated in various illnesses of different organs such as prostate, lungs, CNS, kidneys, pancreas, and skin. In addition to antifibrotic, immunomodulating, and anti-inflammatory effects, silymarin has antioxidant properties by scavenging free radicals and increasing the glutathione concentrations, so that it can be used in hepatitis and hepatic cirrhosis treatment and in mushroom poisoning. See e.g., Karimi G. et al., Iran J Basic Med Sci., 14(4):308-317 (2011).

In certain aspects, the compositions of the present disclosure comprise Schisandra chinensis in the amount of about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, or about 350 mg. In certain aspects, the compositions of the present disclosure comprise about 125 mg of Schisandra chinensis. In certain aspects, the compositions of the present disclosure comprise 125 mg of Schisandra chinensis. In certain aspects, the compositions of the present disclosure comprise about 250 mg of Schisandra chinensis. In certain aspects, the compositions of the present disclosure comprise 250 mg of Schisandra chinensis.

Schisandra (magnolia vine) is a genus of twining shrub in family Schisandraceae, native to Asia and North America, with a center of diversity in China. See e.g., Hutchinson, J., The Families of Flowering Plants, ed. 3. Oxford. pp. 161-162 (1973); Smith, A. C., The families Illiciaceae and Schisandraceae. Sargentia 7:1-224 (1947).

Schisandra species of the present disclosure include, but are not limited to, Schisandra chinensis (magnolia-vine, Chinese magnolia-vine, schisandra, whose fruit is called magnolia berry or five-flavor-fruit; see e.g., Nowak A., et al., Nutrients., 11(2):333 (2019).), Schisandra arisanensis, Schisandra bicolor, Schisandra elongata, Schisandra glabra, Schisandra glaucescens, Schisandra grandiflora, Schisandra henryi, Schisandra incarnata, Schisandra lancifolia, Schisandra macrocarpa, Schisandra micrantha, Schisandra neglecta, Schisandra parapropinqua, Schisandra perulata, Schisandra plena, Schisandra propinqua, Schisandra pubescens, Schisandra pubinervis, Schisandra repanda, Schisandra rubriflora, Schisandra sphaerandra, Schisandra sphenanthera, and Schisandra tomentella.

In certain aspects, the compositions of the present disclosure comprise Cymbopogon flexuosus essential oil in the amount of about 5 mg, about 10 mg, about 15 mg, about 20 mg, about 25 mg, about 30 mg, about 35 mg, about 40 mg, about 45 mg, or about 50 mg. In certain aspects, the compositions of the present disclosure comprise about 15 mg of Cymbopogon flexuosus essential oil. In certain aspects, the compositions of the present disclosure comprise 15 mg of Cymbopogon flexuosus essential oil. In certain aspects, the compositions of the present disclosure comprise about 30 mg of Cymbopogon flexuosus essential oil. In certain aspects, the compositions of the present disclosure comprise 30 mg of Cymbopogon flexuosus essential oil.

Cymbopogon, also known as lemongrass, barbed wire grass, silky heads, Cochin grass, Malabar grass, oily heads or fever grass, is a genus of Asian, African, Australian, and tropical island plants in Poaceae family. See e.g., Soenarko, S., The genus Cymbopogon sprengel (Gramineae). Reinwardtia 9(3):225-375 (1977).

Cymbopogon species of the present disclosure include, but are not limited to, Cymbopogon flexuosus (East Indian lemon grass, see e.g., Han X and Parker T. L., Biochimie Open, 4:107-111 (2017)), Cymbopogon ambiguous, Cymbopogon annamensis, Cymbopogon bhutanicus, Cymbopogon bombycinus (silky oilgrass), Cymbopogon caesius, Cymbopogon calcicola, Cymbopogon calciphilus, Cymbopogon cambogiensis, Cymbopogon citratus (lemon grass or West Indian lemon grass), Cymbopogon clandestinus, Cymbopogon coloratus, Cymbopogon commutatus, Cymbopogon densiflorus, Cymbopogon dependens, Cymbopogon dieterlenii, Cymbopogon distans, Cymbopogon exsertus, Cymbopogon gidarba, Cymbopogon giganteus, Cymbopogon globosus, Cymbopogon goeringii, Cymbopogon gratus, Cymbopogon jwarancusa, Cymbopogon khasianus, Cymbopogon liangshanensis, Cymbopogon mandalaiaensis, Cymbopogon marginatus, Cymbopogon martini (palmarosa), Cymbopogon mekongensis, Cymbopogon microstachys, Cymbopogon microthecus, Cymbopogon minor, Cymbopogon minutiflorus, Cymbopogon nardus (citronella grass), Cymbopogon nervatus, Cymbopogon obtectus (silky heads), Cymbopogon osmastonii, Cymbopogon pendulus, Cymbopogon polyneuros, Cymbopogon pospischilii, Cymbopogon procerus, Cymbopogon pruinosus, Cymbopogon queenslandicus, Cymbopogon quinhonensis, Cymbopogon rectus, Cymbopogon refractus (barbed wire grass), Cymbopogon schoenanthus (camel hay or camel grass), Cymbopogon tortilis, Cymbopogon tungmaiensis, Cymbopogon winterianus (citronella grass), and Cymbopogon xichangensis.

In certain aspects, the compositions of the present disclosure comprise Humulus lupulus in the amount of about 10 mg, about 20 mg, about 30 mg, about 40 mg, about 50 mg, about 60 mg, about 70 mg, about 80 mg, about 90 mg, about 100 mg, about 110 mg, or about 120 mg. In certain aspects, the compositions of the present disclosure comprise about 25 mg of Humulus lupulus. In certain aspects, the compositions of the present disclosure comprise 25 mg of Humulus lupulus. In certain aspects, the compositions of the present disclosure comprise about 50 mg of Humulus lupulus. In certain aspects, the compositions of the present disclosure comprise 50 mg of Humulus lupulus. In certain aspects, the compositions of the present disclosure comprise about 100 mg of Humulus lupulus. In certain aspects, the compositions of the present disclosure comprise 100 mg of Humulus lupulus

Humulus is a small genus of flowering plants in the family Cannabaceae. See e.g., Murakami A., et al., Heredity, 97:66-74 (2006). Humulus species of the present disclosure include, but are not limited to, Humulus lupulus (hops; see e.g., Chadwick L. R. et al., Phytomedicine., 13(1-2):119-131 (2006)), Humulus japonicas, and Humulus yunnanensis.

In certain aspects, the compositions of the present disclosure comprise Grindelia spp. in the amount of about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg. In certain aspects, the compositions of the present disclosure comprise about 75 mg of Grindelia spp. In certain aspects, the compositions of the present disclosure comprise 75 mg of Grindelia spp. In certain aspects, the compositions of the present disclosure comprise about 150 mg of Grindelia spp. In certain aspects, the compositions of the present disclosure comprise 150 mg of Grindelia spp.

Grindelia (gumweed) is a genus of plants native to the Americas belonging to Asteraceae family. See e.g., Bartolia A. and Tortosab R. D., Flora-Morphology, Distribution, Functional Ecology of Plants, 198(2):106-111 (2003). Gumweed can be used for cough, bronchitis, and swelling (inflammation) of the nose, sinuses, and throat. See e.g., Lyle T. J. “Physio-Medical Therapeutics, Materia Medica and Pharmacy.” National Association of Medical Herbalists of Great Britain, Ltd., Medical College of Herbal Practitioners, London (1932).

Grindelia species of the present disclosure include, but are not limited to, Grindelia adenodonta, Grindelia aegialitis, Grindelia aggregate, Grindelia andina, Grindelia anethifolia, Grindelia arizonica, Grindelia boliviana, Grindelia brachystephana, Grindelia buphthalmoides, Grindelia cabrerae, Grindelia chacoensis, Grindelia chiloensis, Grindelia ciliate, Grindelia confuse, Grindelia coronensis, Grindelia covasii, Grindelia decumbens, Grindelia eligulata, Grindelia fraxinipratensis, Grindelia globularifolia, Grindelia glutinosa, Grindelia grandiflora, Grindelia greenmanii, Grindelia havardii, Grindelia hintoniorum, Grindelia hirsutula, Grindelia howellii, Grindelia integrifolia, Grindelia inuloides Grindelia lanceolate, Grindelia linearifolia, Grindelia macvaughii, Grindelia mendocina Grindelia microcephala, Grindelia nelsonii, Grindelia nuda, Grindelia oaxacana, Grindelia obovatifolia, Grindelia oolepis, Grindelia orientalis, Grindelia oxylepis, Grindelia palmeri, Grindelia patagonica, Grindelia prostrata, Grindelia prunelloides, Grindelia puberula, Grindelia pusilla, Grindelia pygmaea, Grindelia ragonesei, Grindelia robinsonii, Grindelia robusta, Grindelia rupestris, Grindelia scabra, Grindelia scorzonerifolia, Grindelia squarrosa, Grindelia stricta, Grindelia subalpine, Grindelia subdecurrens, Grindelia sublanuginosa, Grindelia tarapacana, Grindelia tehuelches, Grindelia tenella, Grindelia turneri, Grindelia ventanensis, Grindelia vetimontis, and Grindelia villarrealii.

In certain aspects, the compositions of the present disclosure comprise Ligusticum wallichii in the amount of about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 175 mg, about 180 mg, about 185 mg, about 190 mg, about 195 mg, or about 200 mg. In certain aspects, the compositions of the present disclosure comprise about 55 mg of Ligusticum wallichii. In certain aspects, the compositions of the present disclosure comprise 55 mg of Ligusticum wallichii. In certain aspects, the compositions of the present disclosure comprise about 110 mg of Ligusticum wallichii. In certain aspects, the compositions of the present disclosure comprise 110 mg of Ligusticum wallichii.

Ligusticum (lovage) is a genus of about 60 species of flowering plants in the family Apiaceae, native to cool temperate regions of the Northern Hemisphere. See e.g., She M et al., Ligusticum Linnaeus, Sp. Pl. 1: 250. 1753 (2005). In Flora of Chinaial Committee; Zhengyi W. and Raven P. (eds.). Apiaceae through Ericaceae. Flora of China. 14. Science Press & Missouri Botanical Garden Press.

Ligusticum species of the present disclosure include, but are not limited to, Ligusticum wallichii (Ligusticum chuanxiong; see e.g., Ran X. et al., Pharmaceutical Biology, 49(11):1180-1189 (2011)), Ligusticum ajanense, Ligusticum apiifolium, Ligusticum brachylobum, Ligusticum caldera, Ligusticum californicum, Ligusticum canadense, Ligusticum canbyi, Ligusticum filicinum, Ligusticum gingidium, Ligusticum grayi, Ligusticum holopetalum, Ligusticum hultenii, Ligusticum huteri, Ligusticum ibukicola, Ligusticum jeholense, Ligusticum monnieri, Ligusticum mutellina (alpine lovage), Ligusticum mutellinoides (small alpine lovage), Ligusticum porteri (osha), Ligusticum scoticum (Scots lovage), Ligusticum sinense (gaoben), Ligusticum striatum (Szechuan lovage, Szechwan lovage, chuanxiong, chuan xiong), Ligusticum tenuifolium (Idaho lovage), Ligusticum vaginatum, and Ligusticum verticillatum (northern lovage).

In certain aspects, the compositions of the present disclosure comprise 5-hydroxytryptophan (5-HTP) in the amount of about 5 mg, about 10 mg, about 15 mg, about 20 mg, about 25 mg, about 30 mg, about 35 mg, about 40 mg, about 45 mg, or about 50 mg. In certain aspects, the compositions of the present disclosure comprise about 55 mg of 5-HTP. In certain aspects, the compositions of the present disclosure comprise 55 mg of 5-HTP. In certain aspects, the compositions of the present disclosure comprise about 110 mg of 5-HTP. In certain aspects, the compositions of the present disclosure comprise 110 mg of 5-HTP.

Hydroxytryptophan (5-HTP) is the intermediate metabolite of the essential amino acid L-tryptophan (LT) in the biosynthesis of serotonin. 5-HTP acts primarily by increasing levels of serotonin within the central nervous system (CNS). Other neurotransmitters and CNS chemicals, such as melatonin, dopamine, norepinephrine, and beta-endorphin have also been shown to increase following oral administration of 5-HTP. 5-HTP can be used for sleep disorders such as insomnia, depression, anxiety, migraine and tension-type headaches, fibromyalgia, obesity, premenstrual syndrome (PMS), premenstrual dysphoric disorder (PMDD), attention deficit-hyperactivity disorder (ADHD), seizure disorder, and Parkinson's disease. See e.g., Birdsall T., Alternative Medicine Review, 3(4):271-280 (1998); Morrow, J. D. et al., Sleep, 31(1), 21-33 (2008); Hong K B. et al., Life Sci., 150:42-49 (2016); Denoyer M. et al., Neuroscience., 28(1):83-94 (1989); Bruni O. et al., Expert Opin Pharmacother. 20(18):2257-2271 (2019); Weeks B S. et al., Med Sci Monit., 15(11):RA256-RA262 (2009); Hong K B et al., Nutr Res Pract. 12(3):208-214 (2018); Touret M et al., Exp Brain Res., 86(1):117-124 (1991); Denoyer M et al., Neuroscience., 28(1):83-94 (1989); Yohn, C. N. et al., Molecular Brain, 10(1):28 (2017); Soderpalm B. et al., Pharmacol Biochem Behav., 32(1):259-265 (1989); Bussone G. et al., Riv Patol Nerv Ment. 100(5):269-274 (1979); Nicolodi M et al., Adv Exp Med Biol., 398:373-379 (1996); Eriksson O. et al., PLoS One., 11(9):e0159538 (2016); Hinz, M. et al, Neuropsychiatric Disease and Treatment, 7:31-38 (2011); Truscott T C. Pharmacol Biochem Behav. 3(5):939-941 (1975); Meloni M et al., Eur J Neurol., 27(5):779-786 (2020).

In certain aspects, the compositions of the present disclosure comprise paeonol in the amount of about 15 mg, about 20 mg, about 25 mg, about 30 mg, about 35 mg, about 40 mg, about 45 mg, or about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg. In certain aspects, the compositions of the present disclosure comprise about 25 mg of paeonol. In certain aspects, the compositions of the present disclosure comprise 25 mg of paeonol. In certain aspects, the compositions of the present disclosure comprise about 50 mg of paeonol. In certain aspects, the compositions of the present disclosure comprise 50 mg of paeonol.

Paeonol, a major phenolic component of Moutan Cortex, the root bark of Paeonia moutan, is widely used as a nutrient supplement in Chinese medicine. It possesses a broad range of properties like inhibiting collagen-induced platelet aggregation and attenuating inflammatory responses in airways, coronary arteries, macrophages and microglia cells Accumulating evidence indicates that paeonol can be a promising neuroprotective or anti-neurodegenerative compound because of its anti-inflammatory and free-radical scavenging properties. See e.g., Lin C., et al., Int J Mol Sci. 16(4):8844-8860 (2015).

In certain aspects, the compositions of the present disclosure comprise Panax ginseng in the amount of about 15 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg. In certain aspects, the compositions of the present disclosure comprise about 50 mg of Panax ginseng. In certain aspects, the compositions of the present disclosure comprise 50 mg of Panax ginseng. In certain aspects, the compositions of the present disclosure comprise about 100 mg of Panax ginseng. In certain aspects, the compositions of the present disclosure comprise 100 mg of Panax ginseng.

The Panax (ginseng) genus belongs to the Araliaceae (ivy) family. See e.g., Boopathi V. et al., J Ginseng Res., 44(1):33-43 (2020). Panax species of the present disclosure include, but are not limited to, Panax ginseng (Asian ginseng, Chinese ginseng, Korean ginseng, Asiatic ginseng, Oriental ginseng; see e.g., Kim J-H., J Ginseng Res., 42(3):264-269 (2018)), Panax notoginseng, Panax bipinnatifidus, Panax japonicas, Panax quinquefolius (American ginseng), Panax sokpayensis, Panax vietnamensis, Panax wangianus, Panax zingiberensis, Panax pseudoginseng, Panax stipuleanatus, and Panax trifolius.

In certain aspects, the compositions of the present disclosure comprise Rehmannia glutinosa in the amount of about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, or about 100 mg. In certain aspects, the compositions of the present disclosure comprise about 37.5 mg of Rehmannia glutinosa. In certain aspects, the compositions of the present disclosure comprise 37.5 mg of Rehmannia glutinosa. In certain aspects, the compositions of the present disclosure comprise about 75 mg of Rehmannia glutinosa. In certain aspects, the compositions of the present disclosure comprise 75 mg of Rehmannia glutinosa.

Rehmannia is a genus of six species of flowering plants in the Orobanchaceae family. See e.g., Zeng H. et al., Genes (Basel)., 8(3):103 (2017). Rehmannia species of the present disclosure include, but are not limited to, Rehmannia glutinosa (see e.g., Dai H. et al., Front Pharmacol., 9:1012 (2018)), Rehmannia chingii, Rehmannia elata, Rehmannia henryi, Rehmannia piasezkii, and Rehmannia solanifolia.

In certain aspects, the compositions of the present disclosure comprise Polygala tenuifolia in the amount of about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg. In certain aspects, the compositions of the present disclosure comprise about 75 mg of Polygala tenuifolia. In certain aspects, the compositions of the present disclosure comprise 75 mg of Polygala tenuifolia. In certain aspects, the compositions of the present disclosure comprise about 150 mg of Polygala tenuifolia. In certain aspects, the compositions of the present disclosure comprise 150 mg of Polygala tenuifolia.

Polygala is a large genus of flowering plants in the family Polygalaceae. See e.g., De Smet P. A. G. M. (1993) Polygala Species. In: De Smet P. A. G. M., Keller K., Hansel R., Chandler R. F. (eds) Adverse Effects of Herbal Drugs 2. Adverse Effects of Herbal Drugs, vol 2. Springer, Berlin, Heidelberg.

Polygala species of the present disclosure include, but are not limited to, Polygala tenuifolia (Chinese senega-root; see e.g., Shin K. Y. et al., Evid Based Complement Alternat Med. 2014:570134 (2014)), Polygala Africana, Polygala alba (white milkwort), Polygala alpestris (alpine milkwort), Polygala amara (bitter milkwort), Polygala amarella (dwarf milkwort), Polygala ambigua (whorled milkwort), Polygala boykinii (Boykin's milkwort), Polygala calcarea (chalk milkwort), Polygala cruciata (drumheads), Polygala curtissii (Curtiss' milkwort), Polygala helenae, Polygala kuriensis, Polygala lewtonii (Lewton's polygala), Polygala lutea (orange milkwort), Polygala myrtifolia (butterfly-bush, parrotbush, myrtle-leaf milkwort, cascade curse, augustusbos), Polygala nana (candyroot), Polygala poaya (Polygala angulate), Polygala quitensis, Polygala sanguinea (purple milkwort, blood milkwort, field milkwort), Polygala senega (Seneca snakeroot, senega), Polygala serpyllifolia (thyme-leaved milkwort), Polygala sinisica, Polygala smallii (tiny milkwort), and Polygala vulgaris (common milkwort).

In certain aspects, the compositions of the present disclosure comprise Lavendula officinalis essential oil (also known as Lavandula angustifolia) in the amount of about 1 mg, about 5 mg, about 10 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In certain aspects, the compositions of the present disclosure comprise about 40 mg of Lavendula officinalis essential oil. In certain aspects, the compositions of the present disclosure comprise 40 mg of Lavendula officinalis essential oil. In certain aspects, the compositions of the present disclosure comprise about 80 mg of Lavendula officinalis. In certain aspects, the compositions of the present disclosure comprise 80 mg of Lavendula officinalis essential oil.

Lavendula (lavender) is a genus of 47 known species of flowering plants in the mint family, Lamiaceae. See e.g., Salehi B. et al., Natural Product Communications, 13(10):1385-1402 (2018). Lavendula species of the present disclosure include, but are not limited to, Lavendula officinalis (Lavandula angustifolia; see e.g., Cardia G., et al., Evid Based Complement Alternat., 2018:1413940 (2018)), Lavandula latifolia, Lavandula lanata, Lavandula dentata L., Lavandula stoechas L., Lavandula pedunculata, and Lavandula viridis.

In certain aspects, the compositions of the present disclosure comprise Scutellaria lateriflora in the amount of about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg. In certain aspects, the compositions of the present disclosure comprise about 75 mg of Scutellaria lateriflora. In certain aspects, the compositions of the present disclosure comprise 75 mg of Scutellaria lateriflora. In certain aspects, the compositions of the present disclosure comprise about 150 mg of Scutellaria lateriflora. In certain aspects, the compositions of the present disclosure comprise 150 mg of Scutellaria lateriflora.

Scutellaria (skullcaps) a genus of flowering plants in the mint family, Lamiaceae. See e.g., Wang Z-L. et al., Pharm Biol., 56(1):465-484 (2018).

Scutellaria species of the present disclosure include, but are not limited to, Scutellaria lateriflora (American skullcap, blue skullcap, Virginian skullcap; see e.g., Awad R., Phytomedicine, 10(8):640-649 (2003)), Scutellaria alabamensis (Alabama skullcap), Scutellaria albida, Scutellaria alborosea, Scutellaria alpina L. (alpine skullcap), Scutellaria altamaha (pineland skullcap), Scutellaria altissima L. (Somerset skullcap, tall skullcap), Scutellaria amoena, Scutellaria anatolica, Scutellaria angustifolia (narrowleaf skullcap), Scutellaria antirrhinoides (nose skullcap), Scutellaria arenicola (Florida scrub skullcap), Scutellaria arguta (Blue Ridge skullcap), Scutellaria atriplicifolia, Scutellaria aurata, Scutellaria baicalensis (Baikal skullcap, Chinese skullcap), Scutellaria barbata (barbed skullcap), Scutellaria bolanderi (Sierra skullcap), Scutellaria brachyspica, Scutellaria brittonii (Britton's skullcap), Scutellaria bushii (Bush's skullcap), Scutellaria caerulea (blue skullcap), Scutellaria californica (California skullcap), Scutellaria cardiophylla (gulf skullcap), Scutellaria columnae, Scutellaria costaricana (scarlet skullcap, Costa Rican skullcap), Scutellaria drummondii, Scutellaria elliptica (hairy skullcap), Scutellaria floridana (Florida skullcap), Scutellaria formosana, Scutellaria galericulata (common skullcap, marsh skullcap), Scutellaria glabriuscula (Georgia skullcap), Scutellaria hastifolia (spear-leaved skullcap), Scutellaria havanensis (Havana skullcap), Scutellaria hirta, Scutellaria hookeri, Scutellaria humilis, Scutellaria incana (downy skullcap, hoary skullcap), Scutellaria indica, Scutellaria integrifolia (helmet flower), Scutellaria incarnata, and Scutellaria laevis (Culberson County skullcap).

In certain aspects, the compositions of the present disclosure comprise Sceletium tortuosum in the amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In certain aspects, the compositions of the present disclosure comprise about 25 mg of Sceletium tortuosum. In certain aspects, the compositions of the present disclosure comprise 25 mg of Sceletium tortuosum. In certain aspects, the compositions of the present disclosure comprise about 50 mg of Sceletium tortuosum. In certain aspects, the compositions of the present disclosure comprise 50 mg of Sceletium tortuosum.

Sceletium is a genus of succulent plants in the Aizoaceae family. Sceletium species of the present disclosure include, but are not limited to, Sceletium tortuosum (see e.g., Terburg D. et al., Neuropsychopharmacology., 38(13):2708-2716 (2013)). Sceletium crassicaule, Sceletium emarcidum, Sceletium exalatum, Sceletium expansum, Sceletium rigidum, Sceletium strictum, and Sceletium varians.

In certain aspects, the compositions of the present disclosure comprise Piper nigrum in the amount of about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.1 mg, about 2.2 mg, about 2.3 mg, about 2.4 mg, about 2.5 mg, about 2.6 mg, about 2.7 mg, about 2.8 mg, about 2.9 mg, about 3 mg, about 3.1 mg, about 3.2 mg, about 3.3 mg, about 3.4 mg, about 3.5 mg, about 3.6 mg, about 3.7 mg, about 3.8 mg, about 3.9 mg, about 4 mg, about 4.1 mg, about 4.2 mg, about 4.3 mg, about 4.4 mg, about 4.5 mg, about 4.6 mg, about 4.7 mg, about 4.8 mg, about 4.9 mg, about 5 mg, about 5.1 mg, about 5.2 mg, about 5.3 mg, about 5.4 mg, about 5.5 mg, about 5.6 mg, about 5.7 mg, about 5.8 mg, about 5.9 mg, about 6 mg, about 6.1 mg, about 6.2 mg, about 6.3 mg, about 6.4 mg, about 6.5 mg, about 6.6 mg, about 6.7 mg, about 6.8 mg, about 6.9 mg, about 7 mg, about 7.1 mg, about 7.2 mg, about 7.3 mg, about 7.4 mg, about 7.5 mg, about 7.6 mg, about 7.7 mg, about 7.8 mg, about 7.9 mg, about 8 mg, about 8.1 mg, about 8.2 mg, about 8.3 mg, about 8.4 mg, about 8.5 mg, about 8.6 mg, about 8.7 mg, about 8.8 mg, about 8.9 mg, about 9 mg, about 9.1 mg, about 9.2 mg, about 9.3 mg, about 9.4 mg, about 9.5 mg, about 9.6 mg, about 9.7 mg, about 9.8 mg, about 9.9 mg, or about 10 mg. In certain aspects, the compositions of the present disclosure comprise about 3 mg of Piper nigrum. In certain aspects, the compositions of the present disclosure comprise 3 mg of Piper nigrum. In certain aspects, the compositions of the present disclosure comprise about 6 mg of Piper nigrum. In certain aspects, the compositions of the present disclosure comprise 6 mg of Piper nigrum.

In certain aspects, the compositions of the present disclosure comprise about 10 mg, about 20 mg, about 30 mg, about 40 mg, about 50 mg, about 60 mg, about 70 mg, about 80 mg, about 90 mg, or about 100 mg of Piper methysticum,

Piper is a genus of 1,000-2,000 species in the family Piperaceae. See e.g., Salehi B et al., Molecules., 7:24(7) (2019). Piper species of the present disclosure include, but are not limited to, Piper nigrum (see e.g., Takooree H et al., Crit Rev Food Sci Nutr. 59(sup1):S210-S24 (2019)), Piper methysticum, Piper abalienatum, Piper abbadianum, Piper abbreviatum, Piper abditum, Piper abellinum, Piper abutilifolium, Piper abutiloides, Piper adamatum, Piper addisonii, Piper adenandrum, Piper aduncum, Piper aequale, Piper aereum, Piper affictum, Piper afflictum, Piper agellifolium, Piper agostiniorum, Piper aguadulcense, Piper alatabaccum, Piper alatipetiolatum, Piper albanense, Piper albert-smithii, Piper albiciliatum, Piper albidum, Piper albogranulatum, Piper albopapillatum, Piper albopilosum, Piper albozonatum, Piper aleyreanum, Piper allardii, Piper alnoides, Piper alveolatum, Piper bahianum, Piper bahiense, Piper bajanum, Piper caballocochanum, Piper cabralanum, Piper dactylostigmum, Piper daedalum, Piper daguanum, Piper ecallosum, Piper echinocaule, Piper factum, Piper faecatum, Piper fagifolium, Piper gabrielianum, Piper galeatum, and Piper galeottianum.

In certain aspects, the compositions of the present disclosure comprise about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg of Magnolia officinalis.

In certain aspects, the compositions of the present disclosure comprise about 15 mg of cannabidiol (CBD), about 2 mg of cannabinol (CBN), about 75 mg of Ziziphus spinosa, about 100 mg of Lycopus spp., about 50 mg of Passiflora incarnata, about 25 mg of Pueraria lobata, about 15 mg of Actaea racemosa, about 25 mg of Humulus lupulus, and about 3 mg of Piper nigrum.

In certain aspects, the compositions of the present disclosure comprise about 30 mg of cannabidiol (CBD), about 4 mg of cannabinol (CBN), about 150 mg of Ziziphus spinosa, about 200 mg of Lycopus spp., about 100 mg of Passiflora incarnata, about 50 mg of Pueraria lobata, about 30 mg of Actaea racemosa, about 50 mg of Humulus lupulus, and about 6 mg of Piper nigrum.

In certain aspects, the compositions of the present disclosure comprise about 15 mg of cannabidiol (CBD), about 5 mg of cannabinol (CBN), about 50 mg of Lycopus spp., about 90 mg of Passiflora incarnata, about 40 mg of Lavendula officinalis essential oil, about 75 mg of Scutellaria lateriflora, about 40 mg of Piper methysticum, and about 3 mg of Piper nigrum.

In certain aspects, the compositions of the present disclosure comprise about 30 mg of cannabidiol (CBD), about 10 mg of cannabinol (CBN), about 100 mg of Lycopus spp., about 180 mg of Passiflora incarnata, about 80 mg of Lavendula officinalis essential oil, about 150 mg of Scutellaria lateriflora, about 80 mg of Piper methysticum, and about 6 mg of Piper nigrum.

In certain aspects, the compositions of the present disclosure comprise about 10 mg of cannabidiol (CBD), about 2.5 mg of cannabigerol (CBG), about 75 mg of Ziziphus spinosa, about 0.5 mg of melatonin, about 50 mg of Pueraria lobata, about 100 mg of Matricaria recutita, about 75 mg of Magnolia officinalis, and about 3 mg of Piper nigrum.

In certain aspects, the compositions of the present disclosure comprise about 20 mg of cannabidiol (CBD), about 5 mg of cannabigerol (CBG), about 150 mg of Ziziphus spinosa, about 1 mg of melatonin, about 100 mg of Pueraria lobata, about 200 mg of Matricaria recutita, about 150 mg of Magnolia officinalis, and about 6 mg of Piper nigrum

In certain aspects, the compositions of the present disclosure comprise about 10 mg of cannabidiol (CBD), about 1 mg of cannabigerol (CBG), about 75 mg of Withania somnifera, about 50 mg of Matricaria recutita, about 50 mg of Ziziphus spinosa, about 75 mg of Polygala tenuifolia, about 0.25 mg of melatonin, about 30 mg of Citrus reticulata (e.g., Citrus reticulata Blanco essential oil), and about 3 mg of Piper nigrum.

In certain aspects, the compositions of the present disclosure comprise about 20 mg of cannabidiol (CBD), about 2 mg of cannabigerol (CBG), about 150 mg of Withania somnifera, about 100 mg of Matricaria recutita, about 100 mg of Ziziphus spinosa, about 150 mg of Polygala tenuifolia, about 0.5 mg of melatonin, about 60 mg of Citrus reticulata (e.g., Citrus reticulate Blanco essential oil), and about 6 mg of Piper nigrum. In certain aspects, the compositions of the present disclosure comprise about 15 mg of cannabidiol (CBD), about 5 mg of cannabinol (CBN), about 0.5 mg of melatonin, about 25 mg of Citrus reticulata (e.g., Citrus reticulata Blanco essential oil), about 50 mg of EPA/DHA, about 75 mg of Withania somnifera, about 15 mg of Cymbopogon flexuosus essential oil, about 50 mg of Humulus lupulus, about 75 mg of Grindelia spp., about 3 mg of mixed Tocopherols, and about 3 mg of Piper nigrum.

In certain aspects, the compositions of the present disclosure comprise about 30 mg of cannabidiol (CBD), about 10 mg of cannabinol (CBN), about 1 mg of melatonin, about 50 mg of Citrus reticulata (e.g., Citrus reticulata Blanco essential oil), about 100 mg of EPA/DHA, about 150 mg of Withania somnifera, about 30 mg of Cymbopogon flexuosus essential oil, about 100 mg of Humulus lupulus, about 150 mg of Grindelia spp., about 6 mg of mixed Tocopherols, and about 6 mg of Piper nigrum.

In certain aspects, the compositions of the present disclosure comprise about 15 mg of cannabidiol (CBD), about 2 mg of cannabinol (CBN), about 1.25 mg of melatonin, about 25 mg of Anemarrhena asphodeloides, about 50 mg of phosphatidylserine, about 25 mg of alpha lipoic acid, about 60 mg of silymarin, about 125 mg of Schisandra chinensis, about 6 mg of Citrus reticulata (e.g., Citrus reticulata Blanco essential oil), and about 3 mg of Piper nigrum.

In certain aspects, the compositions of the present disclosure comprise about 30 mg of cannabidiol (CBD), about 4 mg of cannabinol (CBN), about 2.5 mg of melatonin, about 50 mg of Anemarrhena asphodeloides, about 100 mg of phosphatidylserine, about 50 mg of alpha lipoic acid, about 120 mg of silymarin, about 250 mg of Schisandra chinensis, about 12 mg of Citrus reticulata (e.g., Citrus reticulata Blanco essential oil), and about 6 mg of Piper nigrum.

In certain aspects, the compositions of the present disclosure comprise about 30 mg of cannabidiol (CBD), about 5 mg of cannabinol (CBN), about 12.5 mg of 5-HTP, about 100 mg of Ziziphus spinosa, about 75 mg of Poria cocos, about 55 mg of Ligusticum wallichii, about 8 mg of Sceletium tortuosum, and about 3 mg of Piper nigrum.

In certain aspects, the compositions of the present disclosure comprise about 60 mg of cannabidiol (CBD), about 10 mg of cannabinol (CBN), about 25 mg of 5-HTP, about 200 mg of Ziziphus spinosa, about 150 mg of Poria cocos, about 100 mg of Ligusticum wallichii, about 16 mg of Sceletium tortuosum, and about 6 mg of Piper nigrum.

In certain aspects, the compositions of the present disclosure comprise about 15 mg of cannabidiol (CBD), about 2.5 mg of cannabinol (CBN), about 75 mg of Poria cocos, about 1.5 mg of melatonin, about 75 mg of Polygala tenuifolia, about 50 mg of Panax ginseng, about 37.5 mg of Rehmannia glutinosa, about 25 mg of paeonol, and about 3 mg of Piper nigrum.

In certain aspects, the compositions of the present disclosure comprise about 30 mg of cannabidiol (CBD), about 5 mg of cannabinol (CBN), about 150 mg of Poria cocos, about 3 mg of melatonin, about 150 mg of Polygala tenuifolia, about 100 mg of Panax ginseng, about 75 mg of Rehmannia glutinosa, about 50 mg of paeonol, and about 6 mg of Piper nigrum.

In some aspects, the compositions of the present disclosure further comprise about 5 mg to about 175 mg of medium-chain triglyceride (MCT) oil, about 5 mg to about 100 mg of beeswax (e.g., yellow USP), about 1 mg to about 30 mg sunflower lecithin, about 0 mg to about 30 mg glycerin (e.g., 99.6%, USP, vegetable-based), or any combination thereof.

In some aspects, the compositions of the present disclosure further comprise about 5 mg, about 6 mg, abut 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, or about 175 mg of medium-chain triglyceride (MCT) oil.

In some aspects, the compositions of the present disclosure further comprise about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, or about 100 mg of beeswax (e.g., yellow USP).

In some aspects, the compositions of the present disclosure further comprise about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, or about 30 mg.

In some aspects, the compositions of the present disclosure further comprise about 0 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, or about 30 mg of glycerin (e.g., 99.6%, USP, vegetable-based).

### Compositions for Pain Conditions

In certain aspects, the compositions of the present disclosure can be used for relieving pain. for improving sleep quality, and/or improving anxiety symptoms

In certain aspects, the compositions of the present disclosure comprise:
(i) about 5 mg to about 80 mg of cannabidiol (CBD), about 1 mg to about 30 mg of cannabinol (CBN), and about 1 mg to about 10 mg of Piper nigrum; and
(ii) three or more of the following: about 30 mg to about 180 mg of eicosapentaenoic acid (EPA)/docosahexaenoic acid (DHA), about 80 mg to about 300 mg of Curcuma longa, about 30 mg to about 180 mg of Nigella sativa oil, about 1 mg to about 30 mg of Zanthoxylum spp., about 10 mg to about 80 mg of Citrus reticulata (e.g., Citrus reticulata Blanco essential oil), about 1 mg to about 15 mg of mixed Tocopherols, about 10 mg to about 80 mg of luteolin, about 0.001 mg to about 1 mg of vitamin D, about 150 mg to about 450 mg of Withania somnifera, about 50 mg to about 220 mg of Corydalis yanhusuo, about 1 mg to about 30 mg of Glycyrrhiza glabra, about 1 mg to about 30 mg of Lavandula angustifolia, about 150 mg to about 400 mg of Scutellaria lateriflora, about 30 mg to about 120 mg of coenzyme Q10, about 50 mg to about 250 mg of Angelica sinensis, about 50 mg to about 200 mg of Ligusticum chuanxiong, about 100 mg to about 300 mg of Salix alba, about 1 mg to about 15 mg of zinc, about 100 mg to about 350 mg of Commiphora mukul, about 0.01 mg to about 1 mg of selenium, about 0.001 mg to about 1 mg of iodine, about 10 mg to about 100 mg of Pueraria lobata, about 100 mg to about 350 mg of Viburnum prunifolium, about 50 mg to about 200 mg of Matricaria recutita, about 1 mg to about 15 mg of Lobelia inflata, about 10 mg to about 80 mg of Melissa officinalis, about 10 mg to about 80 mg of Dioscorea villosa, about 10 mg to about 80 mg of Paeonia lactiflora, and about 1 mg to about 200 mg of Zingiber officinalis.

In certain aspects, the compositions of the present disclosure comprise about 10 mg of CBD. In certain aspects, the compositions of the present disclosure comprise 10 mg of CBD. In certain aspects, the compositions of the present disclosure comprise about 15 mg of CBD. In certain aspects, the compositions of the present disclosure comprise 15 mg of CBD. In certain aspects, the compositions of the present disclosure comprise about 20 mg of CBD. In certain aspects, the compositions of the present disclosure comprise 20 mg of CBD. In certain aspects, the compositions of the present disclosure comprise about 25 mg of CBD. In certain aspects, the compositions of the present disclosure comprise 25 mg of CBD. In certain aspects, the compositions of the present disclosure comprise about 30 mg of CBD. In certain aspects, the compositions of the present disclosure comprise 30 mg of CBD. In certain aspects, the compositions of the present disclosure comprise about 40 mg of CBD. In certain aspects, the compositions of the present disclosure comprise 40 mg of CBD. In certain aspects, the compositions of the present disclosure comprise about 50 mg of CBD. In certain aspects, the compositions of the present disclosure comprise 50 mg of CBD.

In certain aspects, the compositions of the present disclosure comprise about 2.5 mg of CBN. In certain aspects, the compositions of the present disclosure comprise 2.5 mg of CBN. In certain aspects, the compositions of the present disclosure comprise about 5 mg of CBN. In certain aspects, the compositions of the present disclosure comprise 5 mg of CBN. In certain aspects, the compositions of the present disclosure comprise about 10 mg of CBN. In certain aspects, the compositions of the present disclosure comprise 10 mg of CBN. In certain aspects, the compositions of the present disclosure comprise about 20 mg of CBN. In certain aspects, the compositions of the present disclosure comprise 20 mg of CBN.

In certain aspects, the compositions of the present disclosure comprise 3 mg of Piper nigrum. In certain aspects, the compositions of the present disclosure comprise about 6 mg of Piper nigrum. In certain aspects, the compositions of the present disclosure comprise 6 mg of Piper nigrum.

In certain aspects, the compositions of the present disclosure comprise about 65 mg of EPA/DHA. In certain aspects, the compositions of the present disclosure comprise 65 mg of EPA/DHA. In certain aspects, the compositions of the present disclosure comprise about 130 mg of EPA/DHA. In certain aspects, the compositions of the present disclosure comprise 130 mg of EPA/DHA.

In certain aspects, the compositions of the present disclosure comprise about 10 mg of Paeonia lactiflora. In certain aspects, the compositions of the present disclosure comprise about 20 mg of Paeonia lactiflora. In certain aspects, the compositions of the present disclosure comprise about 30 mg of Paeonia lactiflora. In certain aspects, the compositions of the present disclosure comprise about 40 mg of Paeonia lactiflora. In certain aspects, the compositions of the present disclosure comprise about 50 mg of Paeonia lactiflora. In certain aspects, the compositions of the present disclosure comprise about 60 mg of Paeonia lactiflora. In certain aspects, the compositions of the present disclosure comprise about 70 mg of Paeonia lactiflora. In certain aspects, the compositions of the present disclosure comprise about 80 mg of Paeonia lactiflora. In certain aspects, the compositions of the present disclosure comprise about 90 mg of Paeonia lactiflora. In certain aspects, the compositions of the present disclosure comprise about 100 mg of Paeonia lactiflora.

In certain aspects, the compositions of the present disclosure comprise Curcuma longa in the amount of about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg. In certain aspects, the compositions of the present disclosure comprise about 120 mg of Curcuma longa. In certain aspects, the compositions of the present disclosure comprise 120 mg of Curcuma longa. In certain aspects, the compositions of the present disclosure comprise about 240 mg of Curcuma longa. In certain aspects, the compositions of the present disclosure comprise 240 mg of Curcuma longa.

Curcuma is a genus of about 100 accepted species in the family Zingiberaceae. Curcuma species of the present disclosure include, but are not limited to, Curcuma longa (see e.g., Rahmani A H et al., Adv Biomed Res., 7:38 (2018)), Curcuma angustifolia, Curcuma aromatica (wild turmeric), Curcuma alismatifolia (Siam tulip), Curcuma amada, Curcuma caesia (black turmeric), and Curcuma comosa.

In certain aspects, the compositions of the present disclosure comprise Nigella sativa oil in the amount of about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, or about 180 mg. In certain aspects, the compositions of the present disclosure comprise about 60 mg of Nigella sativa oil. In certain aspects, the compositions of the present disclosure comprise 60 mg of Nigella sativa oil. In certain aspects, the compositions of the present disclosure comprise about 65 mg of Nigella sativa oil. In certain aspects, the compositions of the present disclosure comprise 65 mg of Nigella sativa oil. In certain aspects, the compositions of the present disclosure comprise about 120 mg of Nigella sativa oil. In certain aspects, the compositions of the present disclosure comprise 120 mg of Nigella sativa oil. In certain aspects, the compositions of the present disclosure comprise about 130 mg of Nigella sativa oil. In certain aspects, the compositions of the present disclosure comprise 130 mg of Nigella sativa oil.

Nigella is a genus of 18 species of annual plants in the family Ranunculaceae, native to southern Europe, north Africa, south and southwest Asia. Common names applied to members of this genus are nigella, devil-in-α-bush or love-in-α-mist. Nigella species of the present disclosure include, but are not limited to, Nigella sativa (see e.g., Ahmad A et al., Asian Pac J Trop Biomed., 3(5):337-352 (2013)), Nigella arvensis, Nigella carpatha, Nigella damascene, Nigella degenii, Nigella deserti, Nigella doerfleri, Nigella elata, Nigella fumariifola, Nigella hispanica, Nigella latisecta, Nigella nigellastrum, Nigella orientalis, Nigella oxypetala, Nigella papillosa, Nigella segetalis, Nigella stricta, and Nigella unguicularis.

In certain aspects, the compositions of the present disclosure comprise Zanthoxylum spp. in the amount of 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.1 mg, about 2.2 mg, about 2.3 mg, about 2.4 mg, about 2.5 mg, about 2.6 mg, about 2.7 mg, about 2.8 mg, about 2.9 mg, about 3 mg, about 3.1 mg, about 3.2 mg, about 3.3 mg, about 3.4 mg, about 3.5 mg, about 3.6 mg, about 3.7 mg, about 3.8 mg, about 3.9 mg, about 4 mg, about 4.1 mg, about 4.2 mg, about 4.3 mg, about 4.4 mg, about 4.5 mg, about 4.6 mg, about 4.7 mg, about 4.8 mg, about 4.9 mg, about 5 mg, about 5.1 mg, about 5.2 mg, about 5.3 mg, about 5.4 mg, about 5.5 mg, about 5.6 mg, about 5.7 mg, about 5.8 mg, about 5.9 mg, about 6 mg, about 6.1 mg, about 6.2 mg, about 6.3 mg, about 6.4 mg, about 6.5 mg, about 6.6 mg, about 6.7 mg, about 6.8 mg, about 6.9 mg, about 7 mg, about 7.1 mg, about 7.2 mg, about 7.3 mg, about 7.4 mg, about 7.5 mg, about 7.6 mg, about 7.7 mg, about 7.8 mg, about 7.9 mg, about 8 mg, about 8.1 mg, about 8.2 mg, about 8.3 mg, about 8.4 mg, about 8.5 mg, about 8.6 mg, about 8.7 mg, about 8.8 mg, about 8.9 mg, about 9 mg, about 9.1 mg, about 9.2 mg, about 9.3 mg, about 9.4 mg, about 9.5 mg, about 9.6 mg, about 9.7 mg, about 9.8 mg, about 9.9 mg, about 10 mg, about 10.1 mg, about 10.2 mg, about 10.3 mg, about 10.4 mg, about 10.5 mg, about 10.6 mg, about 10.7 mg, about 10.8 mg, about 10.9 mg, about 11 mg, about 11.1 mg, about 11.2 mg, about 11.3 mg, about 11.4 mg, about 11.5 mg, about 11.6 mg, about 11.7 mg, about 11.8 mg, about 11.9 mg, about 12 mg, about 12.1 mg, about 12.2 mg, about 12.3 mg, about 12.4 mg, about 12.5 mg, about 12.6 mg, about 12.7 mg, about 12.8 mg, about 12.9 mg, about 13 mg, about 13.1 mg, about 13.2 mg, about 13.3 mg, about 13.4 mg, about 13.5 mg, about 13.6 mg, about 13.7 mg, about 13.8 mg, about 13.9 mg, about 14 mg, about 14.1 mg, about 14.2 mg, about 14.3 mg, about 14.4 mg, about 14.5 mg, about 14.6 mg, about 14.7 mg, about 14.8 mg, about 14.9 mg, about 15 mg, about 20 mg, about 25 mg, or about 30 mg. In certain aspects, the compositions of the present disclosure comprise about 10 mg of Zanthoxylum spp. In certain aspects, the compositions of the present disclosure comprise 10 mg of Zanthoxylum spp. In certain aspects, the compositions of the present disclosure comprise about 20 mg of Zanthoxylum spp. In certain aspects, the compositions of the present disclosure comprise 20 mg of Zanthoxylum spp.

Zanthoxylum is a genus of about 250 species of deciduous and evergreen trees and shrubs in the citrus or rue family, Rutaceae. Zanthoxylum species of the present disclosure include, but are not limited to Zanthoxylum americanum, Zanthoxylum clavaherculis, Zanthoxylum piperitum, and Zanthoxylum bungeanum.

In certain aspects, the compositions of the present disclosure comprise one or more essential oils from Citrus species. In some aspects, the Citrus species is Citrus reticulata (e.g., Citrus reticulata Blanco essential oil). In certain aspects, the compositions of the present disclosure comprise Citrus reticulata (e.g., Citrus reticulata Blanco essential oil) in the amount of about 10 mg, about 10.1 mg, about 10.2 mg, about 10.3 mg, about 10.4 mg, about 10.5 mg, about 10.6 mg, about 10.7 mg, about 10.8 mg, about 10.9 mg, about 11 mg, about 11.1 mg, about 11.2 mg, about 11.3 mg, about 11.4 mg, about 11.5 mg, about 11.6 mg, about 11.7 mg, about 11.8 mg, about 11.9 mg, about 12 mg, about 12.1 mg, about 12.2 mg, about 12.3 mg, about 12.4 mg, about 12.5 mg, about 12.6 mg, about 12.7 mg, about 12.8 mg, about 12.9 mg, about 13 mg, about 13.1 mg, about 13.2 mg, about 13.3 mg, about 13.4 mg, about 13.5 mg, about 13.6 mg, about 13.7 mg, about 13.8 mg, about 13.9 mg, about 14 mg, about 14.1 mg, about 14.2 mg, about 14.3 mg, about 14.4 mg, about 14.5 mg, about 14.6 mg, about 14.7 mg, about 14.8 mg, about 14.9 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, or about 80 mg. In certain aspects, the compositions of the present disclosure comprise about 22.5 mg of Citrus reticulata (e.g., Citrus reticulata Blanco essential oil). In certain aspects, the compositions of the present disclosure comprise 22.5 mg of Citrus reticulata (e.g., Citrus reticulata Blanco essential oil). In certain aspects, the compositions of the present disclosure comprise about 25 mg of Citrus reticulata (e.g., Citrus reticulata Blanco essential oil). In certain aspects, the compositions of the present disclosure comprise 25 mg of Citrus reticulata (e.g., Citrus reticulata Blanco essential oil). In certain aspects, the compositions of the present disclosure comprise about 45 mg of Citrus reticulata (e.g., Citrus reticulata Blanco essential oil). In certain aspects, the compositions of the present disclosure comprise 45 mg of Citrus reticulata (e.g., Citrus reticulata Blanco essential oil). In certain aspects, the compositions of the present disclosure comprise about 50 mg of Citrus reticulata (e.g., Citrus reticulata Blanco essential oil). In certain aspects, the compositions of the present disclosure comprise 50 mg of Citrus reticulata (e.g., Citrus reticulata Blanco essential oil).

Citrus is a genus of flowering trees and shrubs in the citrus or rue family, Rutaceae. Citrus species of the present disclosure include, but are not limited to Citrus reticulata (e.g., Citrus reticulata Blanco) (mandarin orange; see e.g., Apraj V D. et al., Pharmacognosy Res., 8(3):160-168 (2016)), Citrus crenatifolia, Citrus japonica, Citrus mangshanensis, Citrus maxima (pummelo, shaddock), Citrus medica, Citrus platymamma, Citrus sinensis, and Citrus trifoliata. In some aspects, the compositions of the present disclosure comprise Citrus reticulata. In some aspects, Citrus reticulata is Citrus reticulata Blanco. In some aspects, the compositions of the present disclosure comprise Citrus reticulata Blanco essential oil.

In certain aspects, the compositions of the present disclosure comprise mixed Tocopherols in the amount of about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.1 mg, about 2.2 mg, about 2.3 mg, about 2.4 mg, about 2.5 mg, about 2.6 mg, about 2.7 mg, about 2.8 mg, about 2.9 mg, about 3 mg, about 3.1 mg, about 3.2 mg, about 3.3 mg, about 3.4 mg, about 3.5 mg, about 3.6 mg, about 3.7 mg, about 3.8 mg, about 3.9 mg, about 4 mg, about 4.1 mg, about 4.2 mg, about 4.3 mg, about 4.4 mg, about 4.5 mg, about 4.6 mg, about 4.7 mg, about 4.8 mg, about 4.9 mg, about 5 mg, about 5.1 mg, about 5.2 mg, about 5.3 mg, about 5.4 mg, about 5.5 mg, about 5.6 mg, about 5.7 mg, about 5.8 mg, about 5.9 mg, about 6 mg, about 6.1 mg, about 6.2 mg, about 6.3 mg, about 6.4 mg, about 6.5 mg, about 6.6 mg, about 6.7 mg, about 6.8 mg, about 6.9 mg, about 7 mg, about 7.1 mg, about 7.2 mg, about 7.3 mg, about 7.4 mg, about 7.5 mg, about 7.6 mg, about 7.7 mg, about 7.8 mg, about 7.9 mg, about 8 mg, about 8.1 mg, about 8.2 mg, about 8.3 mg, about 8.4 mg, about 8.5 mg, about 8.6 mg, about 8.7 mg, about 8.8 mg, about 8.9 mg, about 9 mg, about 9.1 mg, about 9.2 mg, about 9.3 mg, about 9.4 mg, about 9.5 mg, about 9.6 mg, about 9.7 mg, about 9.8 mg, about 9.9 mg, about 10 mg, about 10.1 mg, about 10.2 mg, about 10.3 mg, about 10.4 mg, about 10.5 mg, about 10.6 mg, about 10.7 mg, about 10.8 mg, about 10.9 mg, about 11 mg, about 11.1 mg, about 11.2 mg, about 11.3 mg, about 11.4 mg, about 11.5 mg, about 11.6 mg, about 11.7 mg, about 11.8 mg, about 11.9 mg, about 12 mg, about 12.1 mg, about 12.2 mg, about 12.3 mg, about 12.4 mg, about 12.5 mg, about 12.6 mg, about 12.7 mg, about 12.8 mg, about 12.9 mg, about 13 mg, about 13.1 mg, about 13.2 mg, about 13.3 mg, about 13.4 mg, about 13.5 mg, about 13.6 mg, about 13.7 mg, about 13.8 mg, about 13.9 mg, about 14 mg, about 14.1 mg, about 14.2 mg, about 14.3 mg, about 14.4 mg, about 14.5 mg, about 14.6 mg, about 14.7 mg, about 14.8 mg, about 14.9 mg, or about 15 mg. In certain aspects, the compositions of the present disclosure comprise about 4 mg of mixed Tocopherols. In certain aspects, the compositions of the present disclosure comprise 4 mg of mixed Tocopherols. In certain aspects, the compositions of the present disclosure comprise about 8 mg of mixed Tocopherols. In certain aspects, the compositions of the present disclosure comprise 8 mg of mixed Tocopherols.

Tocopherols, the major forms of vitamin E, are a group of fat-soluble phenolic compounds. All tocopherols are strong antioxidants. The major dietary sources of tocopherols are vegetable oils such as corn, soybean, sesame, and cottonseed. See e.g., Das Gupta S, and Suh N., Mol Nutr Food Res., 60(6):1354-1363 (2016).

In certain aspects, the compositions of the present disclosure comprise luteolin in the amount of about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, or about 80 mg. In certain aspects, the compositions of the present disclosure comprise about 25 mg of luteolin. In certain aspects, the compositions of the present disclosure comprise 25 mg of luteolin. In certain aspects, the compositions of the present disclosure comprise about 50 mg of luteolin. In certain aspects, the compositions of the present disclosure comprise 50 mg of luteolin.

Luteolin, 3′,4′,5,7-tetrahydroxyflavone, is a common flavonoid that exists in many types of plants including fruits, vegetables, and medicinal herbs. Plants rich in luteolin have been used in Chinese traditional medicine for treating various diseases such as hypertension, inflammatory disorders, and cancer. Having multiple biological effects such as anti-inflammation, antinociceptive (Büyükokuroglu et al., Asian Journal of Chemistry Vol. 20, No. 3 (2008), 1900-1906) anti-allergy and anticancer, luteolin functions as either an antioxidant or a pro-oxidant biochemically. See e.g., Lin Y et al., Curr Cancer Drug Targets., 8(7):634-646 (2008).

In some aspects, the compositions of the present disclosure comprise vitamin D. See e.g, Carlberg, C., et al., PLoS One, 8(7):e71042 (2013); Chen, T. C., et al., J Nutr Biochem, 11(5):267-272 (2013); Bikle D. et al., Chemistry & Biology, 21(3):319-329 (2014); Baeke F et al., Curr Opin Pharmacol., 10(4):482-496 (2010); Prietl, B. et al., Nutrients, 5(7):2502-2521 (2013).

In certain aspects, the compositions of the present disclosure comprise vitamin D in the amount of about 0.001 mg, about 0.002 mg, about 0.003 mg, about 0.004 mg, about 0.005 mg, about 0.006 mg, about 0.007 mg, about 0.008 mg, about 0.009 mg, about 0.01 mg, 0.015 mg, about 0.02 mg, about 0.025 mg, about 0.03 mg, about 0.035 mg, about 0.04 mg, about 0.045 mg, about 0.05 mg, about 0.055 mg, about 0.06 mg, about 0.065 mg, about 0.07 mg, about 0.075 mg, about 0.08 mg, about 0.085 mg, about 0.09 mg, about 0.095 mg, about 0.1 mg, about 0.15 mg, about 0.2 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, or about 1 mg. In certain aspects, the compositions of the present disclosure comprise about 0.025 mg of vitamin D. In certain aspects, the compositions of the present disclosure comprise 0.025 mg of vitamin D. In certain aspects, the compositions of the present disclosure comprise about 0.05 mg of vitamin D. In certain aspects, the compositions of the present disclosure comprise 0.05 mg of vitamin D. In certain aspects, the compositions of the present disclosure comprise about 0.01 mg of vitamin D. In certain aspects, the compositions of the present disclosure comprise 0.01 mg of vitamin D. In certain aspects, the compositions of the present disclosure comprise about 0.02 mg of vitamin D. In certain aspects, the compositions of the present disclosure comprise 0.02 mg of vitamin D.

In certain aspects, the compositions of the present disclosure comprise about 200 mg of Withania somnifera. In certain aspects, the compositions of the present disclosure comprise 200 mg of Withania somnifera. In certain aspects, the compositions of the present disclosure comprise about 400 mg of Withania somnifera. In certain aspects, the compositions of the present disclosure comprise 400 mg of Withania somnifera.

In certain aspects, the compositions of the present disclosure comprise Corydalis yanhusuo in the amount of 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, or about 220 mg. In certain aspects, the compositions of the present disclosure comprise about 75 mg of Corydalis yanhusuo. In certain aspects, the compositions of the present disclosure comprise 75 mg of Corydalis yanhusuo. In certain aspects, the compositions of the present disclosure comprise about 90 mg of Corydalis yanhusuo. In certain aspects, the compositions of the present disclosure comprise 90 mg of Corydalis yanhusuo. In certain aspects, the compositions of the present disclosure comprise about 150 mg of Corydalis yanhusuo. In certain aspects, the compositions of the present disclosure comprise 150 mg of Corydalis yanhusuo. In certain aspects, the compositions of the present disclosure comprise about 180 mg of Corydalis yanhusuo. In certain aspects, the compositions of the present disclosure comprise 180 mg of Corydalis yanhusuo.

Corydalis is a genus of about 470 species of annual and perennial herbaceous plants in the family Papaveraceae. Corydalis species of the present disclosure include, but are not limited to Corydalis yanhusuo, Corydalis decumbens (e.g., Cheng C, et al., Biomed Res Int. 2019; 2019:9614781 (2019)), Corydalis mucronfera (e.g., Zhang J, et al., Phytochemistry, 159:199-207 (2019)), Corydalis bungeana (e.g., Yang C et al., Molecules., 21(8):975 (2016)), Corydalis tomentella (e.g., Wang Y M et al., Ming W Z, Bioorg Chem., 95:103489 (2020)), Corydalis saxicola (e.g., Liu X W et al., J Pharm Biomed Res Int., Biomed Anal. 159:252-261 (2018)), Corydalis ternata (e.g., Kim S R et al., Planta Med., 65(3):218-221 (1999), Corydalis turtschaninovii (e.g., Lee H et al., Int J Mol Sci., 18(12):2748 (2017)), Corydalis govaniana (e.g., Sivakumaran N et al., 2018:3171348 (2018)), Corydalis racemosa (e.g., Yao H N et al., Nat Prod Res. 1-7 (2019)), Corydalis edulis (e.g., Liang S. et al., J Ethnopharmacol., 251:112540 (2020)), Corydalis ambigua var. amurensis (e.g., Zhu X Z. et al., 86 Suppl 2:173-175 (1991)), Corydalis hendersonii (e.g., Bai R et al., J Ethnopharmacol., 207:174-183 (2017)), Corydalis calliantha (e.g., Wangchuk P et al., Phytother Res., 24(4):481-485 (2010)), Corydalis heterocarpa (e.g., Kang K H et al., Food Chem Toxicol., 47(8):2129-2134 (2009)), Corydalis ochotensis (e.g., Yu J J et al., 37(10):1795-1798 (2014)), Corydalis lutea (e.g., Boegge S C et al., Planta Med., 62(2):173-174 (1996)), and Corydalis dubia (e.g., Shepherd C et al., J Ethnopharmacol., 211:17-28 (2018)).

In certain aspects, the compositions of the present disclosure comprise Glycyrrhiza glabra in the amount of about 1 mg to about 30 mg

Glycyrrhiza is a genus of about 20 species in the legume family (Fabaceae). Glycyrrhiza species of the present disclosure include, but are not limited to Glycyrrhiza glabra (e.g., Al-Qarawi A A et al., Food Chem Toxicol., 40(10):1525-1527 (2002)), Glycyrrhiza inflata (e.g., Chang K H et al., Drug Des Devel Ther., 10:885-896 (2016)), and Glycyrrhiza uralensis (e.g., Yang, N. et al., Phytotherapy research: PTR, 27(9): 1381-1391 (2013)).

In certain aspects, the compositions of the present disclosure comprise about 10 mg of Lavendula angustifolia. In certain aspects, the compositions of the present disclosure comprise 10 mg of Lavendula angustifolia. In certain aspects, the compositions of the present disclosure comprise about 20 mg of Lavendula angustifolia. In certain aspects, the compositions of the present disclosure comprise 20 mg of Lavendula angustifolia.

In certain aspects, the compositions of the present disclosure comprise about 175 mg of Scutellaria lateriflora. In certain aspects, the compositions of the present disclosure comprise 175 mg of Scutellaria lateriflora. In certain aspects, the compositions of the present disclosure comprise about 350 mg of Scutellaria lateriflora. In certain aspects, the compositions of the present disclosure comprise 350 mg of Scutellaria lateriflora.

In certain aspects, the compositions of the present disclosure comprise Angelica sinensis in the amount of about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, or about 250 mg. In certain aspects, the compositions of the present disclosure comprise about 100 mg of Angelica sinensis. In certain aspects, the compositions of the present disclosure comprise 100 mg of Angelica sinensis. In certain aspects, the compositions of the present disclosure comprise about 200 mg of Angelica sinensis. In certain aspects, the compositions of the present disclosure comprise 200 mg of Angelica sinensis.

Angelica is a genus of about 60 species of tall biennial and perennial herbs in the family Apiaceae. Angelica species of the present disclosure include, but are not limited to Angelica sinensis (e.g., Ying, Li et al., Pharmacognosy Magazine. 9 (34):114-119 (2013)), Angelica acutiloba, Angelica arguta, Angelica breweri, Angelica callii, Angelica dahurica, Angelica genuflexa, Angelica hendersonii, Angelica kingii, Angelica palustris, and Angelica scabrida.

In certain aspects, the compositions of the present disclosure comprise about 80 mg of Ligusticum chuanxiong. In certain aspects, the compositions of the present disclosure comprise 80 mg of Ligusticum chuanxiong. In certain aspects, the compositions of the present disclosure comprise about 160 mg of Ligusticum chuanxiong. In certain aspects, the compositions of the present disclosure comprise 160 mg of Ligusticum chuanxiong.

In certain aspects, the compositions of the present disclosure comprise Salix alba in the amount of about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg. In certain aspects, the compositions of the present disclosure comprise about 125 mg of Salix alba. In certain aspects, the compositions of the present disclosure comprise 125 mg of Salix alba. In certain aspects, the compositions of the present disclosure comprise about 250 mg of Salix alba. In certain aspects, the compositions of the present disclosure comprise 250 mg of Salix alba.

Salix is a genus of about 400 species of deciduous trees and shrubs in the family Salicaceae. Salix species of the present disclosure include, but are not limited to Salix alba (e.g., Morader, H. L. et al., Annals of Botany., 86(5): 1017-1021 (2000)), Salix cordata, Salix interior, Salix nakamurana, Salix serissima, and Salix yoshinoi.

In certain aspects, the compositions of the present disclosure comprise Commiphora mukul in the amount of about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, or about 350 mg. In certain aspects, the compositions of the present disclosure comprise about 150 mg of Commiphora mukul. In certain aspects, the compositions of the present disclosure comprise 150 mg of Commiphora mukul. In certain aspects, the compositions of the present disclosure comprise about 300 mg of Commiphora mukul. In certain aspects, the compositions of the present disclosure comprise 300 mg of Commiphora mukul.

Commiphora is a genus of flowering plants in the frankincense and myrrh family, Burseraceae. Commiphora species of the present disclosure include, but are not limited to Commiphora mukul (e.g., Bhatia et al., Phytochemistry., 110:29-36 (2015)), Commiphora angolensis, Commiphora caudata, Commiphora gileadensis, Commiphora habessinica, Commiphora myrrha, Commiphora saxicola, Commiphora schimperi, Commiphora stocksiana, and Commiphora unilobata.

In certain aspects, the compositions of the present disclosure comprise Viburnum prunifolium in the amount of about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, or about 350 mg. In certain aspects, the compositions of the present disclosure comprise about 150 mg of Viburnum prunifolium. In certain aspects, the compositions of the present disclosure comprise 150 mg of Viburnum prunifolium. In certain aspects, the compositions of the present disclosure comprise about 300 mg of Viburnum prunifolium. In certain aspects, the compositions of the present disclosure comprise 300 mg of Viburnum prunifolium.

Viburnum is a genus of about 150-175 species of flowering plants in the moschatel family Adoxaceae. Viburnum species of the present disclosure include, but are not limited to Viburnum prunifolium (e.g., Michael Castleman. Healing Herbs: The Ultimate Guide to the Curative Power of Nature's Medicine. Rodale Press. pp. 79-81 (1991)), Viburnum cassinoides, Viburnum elatum, Viburnum lentago, Viburnum nudum, Viburnum obovatum, Viburnum opulus and Viburnum rufidulum.

In certain aspects, the compositions of the present disclosure comprise coenzyme Q10 in the amount of about 30 mg, about 40 mg, about 50 mg, about 60 mg, about 70 mg, about 80 mg, about 90 mg, about 100 mg, about 110 mg, or about 120 mg. In certain aspects, the compositions of the present disclosure comprise about 50 mg of coenzyme Q10. In certain aspects, the compositions of the present disclosure comprise 50 mg of coenzyme Q10. In certain aspects, the compositions of the present disclosure comprise about 100 mg of coenzyme Q10. In certain aspects, the compositions of the present disclosure comprise 100 mg of coenzyme Q10. See e.g., Tian, G., et al., Antioxid Redox Signal, 20(16):2606-2620 (2014); Jin, G., et al., J Clin Biochem Nutr, 42(2):167-174 (2008); Tiefenbach, J., et al., Dis Model Mech, 11(9) (2018).

In certain aspects, the compositions of the present disclosure comprise zinc in the amount of about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.1 mg, about 2.2 mg, about 2.3 mg, about 2.4 mg, about 2.5 mg, about 2.6 mg, about 2.7 mg, about 2.8 mg, about 2.9 mg, about 3 mg, about 3.1 mg, about 3.2 mg, about 3.3 mg, about 3.4 mg, about 3.5 mg, about 3.6 mg, about 3.7 mg, about 3.8 mg, about 3.9 mg, about 4 mg, about 4.1 mg, about 4.2 mg, about 4.3 mg, about 4.4 mg, about 4.5 mg, about 4.6 mg, about 4.7 mg, about 4.8 mg, about 4.9 mg, about 5 mg, about 5.1 mg, about 5.2 mg, about 5.3 mg, about 5.4 mg, about 5.5 mg, about 5.6 mg, about 5.7 mg, about 5.8 mg, about 5.9 mg, about 6 mg, about 6.1 mg, about 6.2 mg, about 6.3 mg, about 6.4 mg, about 6.5 mg, about 6.6 mg, about 6.7 mg, about 6.8 mg, about 6.9 mg, about 7 mg, about 7.1 mg, about 7.2 mg, about 7.3 mg, about 7.4 mg, about 7.5 mg, about 7.6 mg, about 7.7 mg, about 7.8 mg, about 7.9 mg, about 8 mg, about 8.1 mg, about 8.2 mg, about 8.3 mg, about 8.4 mg, about 8.5 mg, about 8.6 mg, about 8.7 mg, about 8.8 mg, about 8.9 mg, about 9 mg, about 9.1 mg, about 9.2 mg, about 9.3 mg, about 9.4 mg, about 9.5 mg, about 9.6 mg, about 9.7 mg, about 9.8 mg, about 9.9 mg, about 10 mg, about 10.1 mg, about 10.2 mg, about 10.3 mg, about 10.4 mg, about 10.5 mg, about 10.6 mg, about 10.7 mg, about 10.8 mg, about 10.9 mg, about 11 mg, about 11.1 mg, about 11.2 mg, about 11.3 mg, about 11.4 mg, about 11.5 mg, about 11.6 mg, about 11.7 mg, about 11.8 mg, about 11.9 mg, about 12 mg, about 12.1 mg, about 12.2 mg, about 12.3 mg, about 12.4 mg, about 12.5 mg, about 12.6 mg, about 12.7 mg, about 12.8 mg, about 12.9 mg, about 13 mg, about 13.1 mg, about 13.2 mg, about 13.3 mg, about 13.4 mg, about 13.5 mg, about 13.6 mg, about 13.7 mg, about 13.8 mg, about 13.9 mg, about 14 mg, about 14.1 mg, about 14.2 mg, about 14.3 mg, about 14.4 mg, about 14.5 mg, about 14.6 mg, about 14.7 mg, about 14.8 mg, about 14.9 mg, or about 15 mg. In certain aspects, the compositions of the present disclosure comprise about 5 mg of zinc. In certain aspects, the compositions of the present disclosure comprise 5 mg of zinc. In certain aspects, the compositions of the present disclosure comprise about 10 mg of zinc. In certain aspects, the compositions of the present disclosure comprise 10 mg of zinc.

In certain aspects, the compositions of the present disclosure comprise selenium in the amount of about 0.001 mg, about 0.002 mg, about 0.003 mg, about 0.004 mg, about 0.005 mg, about 0.006 mg, about 0.007 mg, about 0.008 mg, about 0.009 mg, about 0.01 mg, 0.015 mg, about 0.02 mg, about 0.025 mg, about 0.03 mg, about 0.035 mg, about 0.04 mg, about 0.045 mg, about 0.05 mg, about 0.055 mg, about 0.06 mg, about 0.065 mg, about 0.07 mg, about 0.075 mg, about 0.08 mg, about 0.085 mg, about 0.09 mg, about 0.095 mg, about 0.1 mg, about 0.15 mg, about 0.2 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, or about 1 mg. In certain aspects, the compositions of the present disclosure comprise about 0.1 mg of selenium. In certain aspects, the compositions of the present disclosure comprise 0.1 mg of selenium. In certain aspects, the compositions of the present disclosure comprise about 0.2 mg of selenium. In certain aspects, the compositions of the present disclosure comprise 0.2 mg of selenium.

In certain aspects, the compositions of the present disclosure comprise iodine in the amount of about 0.001 mg, about 0.002 mg, about 0.003 mg, about 0.004 mg, about 0.005 mg, about 0.006 mg, about 0.007 mg, about 0.008 mg, about 0.009 mg, about 0.01 mg, 0.015 mg, about 0.02 mg, about 0.025 mg, about 0.03 mg, about 0.035 mg, about 0.04 mg, about 0.045 mg, about 0.05 mg, about 0.055 mg, about 0.06 mg, about 0.065 mg, about 0.07 mg, about 0.075 mg, about 0.08 mg, about 0.085 mg, about 0.09 mg, about 0.095 mg, about 0.1 mg, about 0.15 mg, about 0.2 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, or about 1 mg. In certain aspects, the compositions of the present disclosure comprise about 0.075 mg of iodine. In certain aspects, the compositions of the present disclosure comprise 0.075 mg of iodine. In certain aspects, the compositions of the present disclosure comprise about 0.15 mg of iodine. In certain aspects, the compositions of the present disclosure comprise 0.15 mg of iodine.

In certain aspects, the compositions of the present disclosure comprise about 25 mg of Pueraria lobata. In certain aspects, the compositions of the present disclosure comprise 25 mg of Pueraria lobata. In certain aspects, the compositions of the present disclosure comprise about 50 mg of Pueraria lobata. In certain aspects, the compositions of the present disclosure comprise 50 mg of Pueraria lobata.

In certain aspects, the compositions of the present disclosure comprise about 75 mg of Matricaria recutita. In certain aspects, the compositions of the present disclosure comprise 75 mg of Matricaria recutita. In certain aspects, the compositions of the present disclosure comprise about 150 mg of Matricaria recutita. In certain aspects, the compositions of the present disclosure comprise 150 mg of Matricaria recutita.

In certain aspects, the compositions of the present disclosure comprise Lobelia inflata in the amount of about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.1 mg, about 2.2 mg, about 2.3 mg, about 2.4 mg, about 2.5 mg, about 2.6 mg, about 2.7 mg, about 2.8 mg, about 2.9 mg, about 3 mg, about 3.1 mg, about 3.2 mg, about 3.3 mg, about 3.4 mg, about 3.5 mg, about 3.6 mg, about 3.7 mg, about 3.8 mg, about 3.9 mg, about 4 mg, about 4.1 mg, about 4.2 mg, about 4.3 mg, about 4.4 mg, about 4.5 mg, about 4.6 mg, about 4.7 mg, about 4.8 mg, about 4.9 mg, about 5 mg, about 5.1 mg, about 5.2 mg, about 5.3 mg, about 5.4 mg, about 5.5 mg, about 5.6 mg, about 5.7 mg, about 5.8 mg, about 5.9 mg, about 6 mg, about 6.1 mg, about 6.2 mg, about 6.3 mg, about 6.4 mg, about 6.5 mg, about 6.6 mg, about 6.7 mg, about 6.8 mg, about 6.9 mg, about 7 mg, about 7.1 mg, about 7.2 mg, about 7.3 mg, about 7.4 mg, about 7.5 mg, about 7.6 mg, about 7.7 mg, about 7.8 mg, about 7.9 mg, about 8 mg, about 8.1 mg, about 8.2 mg, about 8.3 mg, about 8.4 mg, about 8.5 mg, about 8.6 mg, about 8.7 mg, about 8.8 mg, about 8.9 mg, about 9 mg, about 9.1 mg, about 9.2 mg, about 9.3 mg, about 9.4 mg, about 9.5 mg, about 9.6 mg, about 9.7 mg, about 9.8 mg, about 9.9 mg, about 10 mg, about 10.1 mg, about 10.2 mg, about 10.3 mg, about 10.4 mg, about 10.5 mg, about 10.6 mg, about 10.7 mg, about 10.8 mg, about 10.9 mg, about 11 mg, about 11.1 mg, about 11.2 mg, about 11.3 mg, about 11.4 mg, about 11.5 mg, about 11.6 mg, about 11.7 mg, about 11.8 mg, about 11.9 mg, about 12 mg, about 12.1 mg, about 12.2 mg, about 12.3 mg, about 12.4 mg, about 12.5 mg, about 12.6 mg, about 12.7 mg, about 12.8 mg, about 12.9 mg, about 13 mg, about 13.1 mg, about 13.2 mg, about 13.3 mg, about 13.4 mg, about 13.5 mg, about 13.6 mg, about 13.7 mg, about 13.8 mg, about 13.9 mg, about 14 mg, about 14.1 mg, about 14.2 mg, about 14.3 mg, about 14.4 mg, about 14.5 mg, about 14.6 mg, about 14.7 mg, about 14.8 mg, about 14.9 mg, or about 15 mg. In certain aspects, the compositions of the present disclosure comprise about 5 mg of Lobelia inflata. In certain aspects, the compositions of the present disclosure comprise 5 mg of Lobelia inflata. In certain aspects, the compositions of the present disclosure comprise about 10 mg of Lobelia inflata. In certain aspects, the compositions of the present disclosure comprise 10 mg of Lobelia inflata.

Lobelia is a genus of about 415 species of flowering plants in the family Campanulaceae. Lobelia species of the present disclosure include, but are not limited to Lobelia inflata (see e.g., Hamann S R et al., Pharmacol Biochem Behav. 47(1):197-201 (1994); King, J., Felter, H. W., & Lloyd, J. U. (1909). King's American dispensatory. Cincinnati: Ohio Valley Co.), Lobelia chinensis, Lobelia kalmia, Lobelia laxiflora, Lobelia gibbosa, Lobelia hereroensis, Lobelia heterophylla, Lobelia pyramidalis, and Lobelia hypoleuca.

In certain aspects, the compositions of the present disclosure comprise Melissa officinalis in the amount of about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 68 mg, about 59 mg, about 60 mg, about 61 mg, about 62 mg, about 63 mg, about 64 mg, about 65 mg, about 66 mg, about 67 mg, about 68 mg, about 69, mg, about 70 mg, about 71 mg, about 72 mg, about 73 mg, about 74 mg, about 75 mg, about 76 mg, about 77 mg, about 78 mg, about 79 mg, or about 80 mg. In certain aspects, the compositions of the present disclosure comprise about 25 mg of Melissa officinalis. In certain aspects, the compositions of the present disclosure comprise 25 mg ofMelissa officinalis. In certain aspects, the compositions of the present disclosure comprise about 50 mg ofMelissa officinalis. In certain aspects, the compositions of the present disclosure comprise 50 mg of Melissa officinalis.

Melissa is a genus of perennial herbs in the family Lamiaceae. Melissa species of the present disclosure include, but are not limited to Melissa officinalis (e.g., Shakeri A. at al., J Ethnopharmacol. 188:204-228 (2016)).

In certain aspects, the compositions of the present disclosure comprise Dioscorea villosa in the amount of about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 68 mg, about 59 mg, about 60 mg, about 61 mg, about 62 mg, about 63 mg, about 64 mg, about 65 mg, about 66 mg, about 67 mg, about 68 mg, about 69, mg, about 70 mg, about 71 mg, about 72 mg, about 73 mg, about 74 mg, about 75 mg, about 76 mg, about 77 mg, about 78 mg, about 79 mg, or about 80 mg. In certain aspects, the compositions of the present disclosure comprise about 25 mg of Dioscorea villosa. In certain aspects, the compositions of the present disclosure comprise 25 mg of Dioscorea villosa. In certain aspects, the compositions of the present disclosure comprise about 50 mg of Dioscorea villosa. In certain aspects, the compositions of the present disclosure comprise 50 mg of Dioscorea villosa.

Dioscorea is a genus of over 600 species of flowering plants in the family Dioscoreaceae. Dioscorea species of the present disclosure include, but are not limited to Dioscorea villosa (e.g., Zhao W X et al., Mol Med Rep. 16(2):1559-1564 (2017)), Dioscorea bulbifera (e.g., Mbiantcha M et al., Alternat Med. 2011:912935 (2011)), Dioscorea spongiosa (e.g., Yin J et al., Planta Med., 74(12):1451-1453 (2008)), Dioscorea nipponica (e.g., Zhou Q et al., J Ethnopharmacol., 206:274-282 (2017), Dioscorea zingiberensis (e.g., Zhang et al., Planta Med., 80(17):1597-1604 (2014)), Dioscorea opposite (e.g., Zeng M. et al., Molecules., 23(2):11 (2018)), Dioscorea collettii (e.g., Zhu L et al., Biomed Pharmacother., 93:88-94 (2017), Dioscorea japonica (e.g., Kim N et al., J Ethnopharmacol., 137(1):312-319 (2011)), Dioscorea alata (e.g., Chen T et al., Food Funct., 8(2):659-669 (2017), and Dioscorea tokoro (e.g., Kim M J et al., Int Immunopharmacol, 4(12):1489-1497 (2004)).

In certain aspects, the compositions of the present disclosure comprise Zingiber officinalis in the amount of and about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 68 mg, about 59 mg, about 60 mg, about 61 mg, about 62 mg, about 63 mg, about 64 mg, about 65 mg, about 66 mg, about 67 mg, about 68 mg, about 69, mg, about 70 mg, about 71 mg, about 72 mg, about 73 mg, about 74 mg, about 75 mg, about 76 mg, about 77 mg, about 78 mg, about 79 mg, or about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg. In certain aspects, the compositions of the present disclosure comprise about 10 mg of Zingiber officinalis. In certain aspects, the compositions of the present disclosure comprise 10 mg of Zingiber officinalis. In certain aspects, the compositions of the present disclosure comprise about 20 mg of Zingiber officinalis. In certain aspects, the compositions of the present disclosure comprise 20 mg of Zingiber officinalis. In certain aspects, the compositions of the present disclosure comprise about 75 mg of Zingiber officinalis. In certain aspects, the compositions of the present disclosure comprise 75 mg of Zingiber officinalis. In certain aspects, the compositions of the present disclosure comprise about 150 mg of Zingiber officinalis. In certain aspects, the compositions of the present disclosure comprise 150 mg of Zingiber officinalis.

Zingiber is a genus of ginger plant species in the family Zingiberaceae. Zingiber species of the present disclosure include, but are not limited to Zingiber officinalis (e.g., Wang J et al., Ann N Y Acad Sci., 1398(1):83-98 (2017)), Zingiber zerumbet, Zingiber mioga, and Zingiber aromaticum.

In certain aspects, the compositions of the present disclosure comprise about 20 mg of cannabidiol (CBD), about 10 mg of cannabinol (CBN), about 3 mg of Piper nigrum, about 65 mg of eicosapentaenoic acid (EPA)/docosahexaenoic acid (DHA), about 120 mg of Curcuma longa, about 65 mg of Nigella sativa oil, about 10 mg of Zanthoxylum spp., about 22.5 mg of Citrus reticulata (e.g., Citrus reticulata Blanco essential oil), and about 4 mg of mixed Tocopherols.

In certain aspects, the compositions of the present disclosure comprise about 40 mg of cannabidiol (CBD), about 20 mg of cannabinol (CBN), about 6 mg of Piper nigrum, about 130 mg of eicosapentaenoic acid (EPA)/docosahexaenoic acid (DHA), about 240 mg of Curcuma longa, about 130 mg of Nigella sativa oil, about 20 mg of Zanthoxylum spp., about 45 mg of Citrus reticulata (e.g., Citrus reticulata Blanco essential oil), and about 8 mg of mixed Tocopherols.

In certain aspects, the compositions of the present disclosure comprise about 10 mg of cannabidiol (CBD), about 2.5 mg of cannabinol (CBN), about 3 mg of Piper nigrum, about 65 mg eicosapentaenoic acid (EPA)/docosahexaenoic acid (DHA), about 120 mg of Curcuma longa, about 60 mg of Nigella sativa oil, about 10 mg of Zanthoxylum spp., about 25 mg of Citrus reticulata (e.g., Citrus reticulata Blanco essential oil), about 4 mg of mixed Tocopherols, about 25 mg of luteolin, and about 0.025 mg of vitamin D.

In certain aspects, the compositions of the present disclosure comprise about 20 mg of cannabidiol (CBD), about 5 mg of cannabinol (CBN), about 6 mg of Piper nigrum, about 130 mg eicosapentaenoic acid (EPA)/docosahexaenoic acid (DHA), about 240 mg of Curcuma longa, about 120 mg of Nigella sativa oil, about 20 mg of Zanthoxylum spp., about 50 mg of Citrus reticulata (e.g., Citrus reticulata Blanco essential oil), about 8 mg of mixed Tocopherols, about 50 mg of luteolin, and about 0.05 mg of vitamin D.

In certain aspects, the compositions of the present disclosure comprise about 25 mg of cannabidiol (CBD), about 5 mg of cannabinol (CBN), about 3 mg of Piper nigrum, about 200 mg of Withania somnifera, about 75 mg of Corydalis yanhusuo, and about 10 mg of Glycyrrhiza glabra.

In certain aspects, the compositions of the present disclosure comprise about 50 mg of cannabidiol (CBD), about 10 mg of cannabinol (CBN), about 6 mg of Piper nigrum, about 400 mg of Withania somnifera, about 150 mg of Corydalis yanhusuo, and about 20 mg of Glycyrrhiza glabra.

In certain aspects, the compositions of the present disclosure comprise about 15 mg of cannabidiol (CBD), about 5 mg of cannabinol (CBN), about 3 mg of Piper nigrum, about 10 mg of Lavandula angustifolia, about 75 mg of Zingiber officinalis, about 175 mg of Scutellaria lateriflora, and about 50 mg of coenzyme Q10.

In certain aspects, the compositions of the present disclosure comprise about 30 mg of cannabidiol (CBD), about 10 mg of cannabinol (CBN), about 6 mg of Piper nigrum, about 20 mg of Lavandula angustifolia, about 150 mg of Zingiber officinalis, about 350 mg of Scutellaria lateriflora, and about 100 mg of coenzyme Q10.

In certain aspects, the compositions of the present disclosure comprise about 15 mg of cannabidiol (CBD), about 5 mg of cannabinol (CBN), about 3 mg of Piper nigrum, about 25 mg of luteolin, about 100 mg of Angelica sinensis, about 80 mg of Ligusticum chuanxiong, and about 90 mg of Corydalis yanhusuo.

In certain aspects, the compositions of the present disclosure comprise about 30 mg of cannabidiol (CBD), about 10 mg of cannabinol (CBN), about 6 mg of Piper nigrum, about 50 mg of luteolin, about 200 mg of Angelica sinensis, about 160 mg of Ligusticum chuanxiong, and about 180 mg of Corydalis yanhusuo.

In certain aspects, the compositions of the present disclosure comprise about 15 mg of cannabidiol (CBD), about 5 mg of cannabinol (CBN), about 3 mg of Piper nigrum, about 125 mg of Salix alba, about 5 mg of zinc, about 0.01 mg of vitamin D, about 150 mg of Commiphora mukul, about 0.1 mg of selenium, and about 0.075 mg of iodine.

In certain aspects, the compositions of the present disclosure comprise about 30 mg of cannabidiol (CBD), about 10 mg of cannabinol (CBN), about 6 mg of Piper nigrum, about 250 mg of Salix alba, about 10 mg of zinc, about 0.02 mg of vitamin D, about 300 mg of Commiphora mukul, about 0.2 mg of selenium, and about 0.15 mg of iodine.

In certain aspects, the compositions of the present disclosure comprise about 15 mg of cannabidiol (CBD), about 2.5 mg of cannabinol (CBN), about 3 mg of Piper nigrum, about 125 mg of Salix alba, about 5 mg of zinc, about 0.01 mg of vitamin D, about 150 mg of Commiphora mukul, about 0.1 mg of selenium, about 0.075 mg of iodine, and about 25 mg of Pueraria lobata.

In certain aspects, the compositions of the present disclosure comprise about 30 mg of cannabidiol (CBD), about 5 mg of cannabinol (CBN), about 6 mg of Piper nigrum, about 250 mg of Salix alba, about 10 mg of zinc, about 0.02 mg of vitamin D, about 300 mg of Commiphora mukul, about 0.2 mg of selenium, about 0.15 mg of iodine, and about 50 mg of Pueraria lobata.

In certain aspects, the compositions of the present disclosure comprise about 15 mg of cannabidiol (CBD), about 5 mg of cannabinol (CBN), about 3 mg of Piper nigrum, about 150 mg of Viburnum prunifolium, about 75 mg of Matricaria recutita, about 5 mg of Lobelia inflata, about 25 mg of Melissa officinalis, about 25 mg of Dioscorea villosa, about 30 mg of Paeonia lactiflora, and about 10 mg of Zingiber officinalis.

In certain aspects, the compositions of the present disclosure comprise about 30 mg of cannabidiol (CBD), about 10 mg of cannabinol (CBN), about 6 mg of Piper nigrum, about 300 mg of Viburnum prunfolium, about 150 mg of Matricaria recutita, about 10 mg of Lobelia inflata, about 50 mg of Melissa officinalis, about 50 mg of Dioscorea villosa, about 60 mg of Paeonia lactiflora, and about 20 mg of Zingiber officinalis.

In some aspects, the compositions of the present disclosure further comprise about 5 mg to about 175 mg of medium-chain triglyceride (MCT) oil, about 5 mg to about 100 mg of beeswax (e.g., yellow USP), about 1 mg to about 30 mg sunflower lecithin, about 0 mg to about 30 mg glycerin (e.g., 99.6%, USP, vegetable-based), or any combination thereof.

In some aspects, the compositions of the present disclosure further comprise about 5 mg, about 6 mg, abut 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, or about 175 mg of medium-chain triglyceride (MCT) oil.

In some aspects, the compositions of the present disclosure further comprise about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, or about 100 mg of beeswax (e.g., yellow USP).

In some aspects, the compositions of the present disclosure further comprise about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, or about 30 mg of sunflower lecithin.

In some aspects, the compositions of the present disclosure further comprise about 0 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, or about 30 mg of glycerin (e.g., 99.6%, USP, vegetable-based).

### Compositions for Anxiety Conditions

In certain aspects, the compositions of the present disclosure can be used for improving anxiety symptoms.

In certain aspects, the compositions of the present disclosure comprise:
(i) about 5 mg to about 80 mg of cannabidiol (CBD), about 1 mg to about 30 mg of cannabinol (CBN), about 1 mg to about 30 mg of cannabigerol (CBG), or any combinations thereof;
(ii) one or more of the following: about 1 mg to about 10 mg of Piper nigrum and about 10 mg to about 150 mg of Citrus reticulata (e.g., Citrus reticulata Blanco essential oil); and
(iiii) two or more of the following: about 50 mg to about 200 mg Lavandula officinalis essential oil, about 10 mg to about 250 mg of Polygala tenuifolia, about 100 mg to about 450 mg of Withania somnifera, about 30 mg to about 150 mg of rutaecarpine, about 30 mg to about 150 mg of Passiflora incarnata, about 100 mg to about 400 mg of Ganoderma lucidum, about 50 mg to about 250 mg of Matricaria recutita, about 10 mg to about 150 mg of Pueraria lobata, about 40 mg to 300 mg of Ziziphus spinosa, about 10 mg to about 100 mg of Sceletium tortuosum, about 5 mg to about 50 mg of Crocus sativus, about 100 mg to about 350 mg of Schisandra chinensis, about 80 mg to about 300 mg of dihydromyricetin, about 5 mg to about 80 mg of 5-hydroxytryptophan (5-HTP), about 50 mg to about 300 mg of Poria cocos, about 20 mg to about 200 mg of Panax ginseng, about 100 mg to about 400 mg of Centella asiatica, about 10 mg to about 200 mg of Magnolia officinalis, about 20 mg to about 100 mg of Piper methysticum, and about 30 mg to about 150 mg of paeonol.

In certain aspects, the compositions of the present disclosure comprise:
(i) about 5 mg to about 80 mg of cannabidiol (CBD), about 1 mg to about 30 mg of cannabinol (CBN), about 1 mg to about 30 mg of cannabigerol (CBG), or any combinations thereof, and
(ii) three or more of the following: about 1 mg to about 10 mg of Piper nigrum about 30 mg to about 150 mg of Citrus reticulata (e.g., Citrus reticulata Blanco essential oil), about 50 mg to about 200 mg Lavandula officinalis, about 10 mg to about 250 mg of Polygala tenuifolia, about 100 mg to about 450 mg of Withania somnifera, about 30 mg to about 150 mg of rutaecarpine, about 10 mg to about 150 mg of Passiflora incarnata, about 100 mg to about 400 mg of Ganoderma lucidum, about 50 mg to about 250 mg of Matricaria recutita, about 10 mg to about 150 mg of Pueraria lobata, about 40 mg to 300 mg of Ziziphus spinosa, about 10 mg to about 100 mg of Sceletium tortuosum, about 5 mg to about 50 mg of Crocus sativus, about 100 mg to about 350 mg of Schisandra chinensis, about 80 mg to about 300 mg of dihydromyricetin, about 5 mg to about 80 mg of 5-hydroxytryptophan (5-HTP), about 50 mg to about 300 mg of Poria cocos, about 20 mg to about 200 mg of Panax ginseng, about 100 mg to about 400 mg of Centella asiatica, about 10 mg to about 200 mg of Magnolia officinalis, about 20 mg to about 100 mg of Piper methysticum, and about 30 mg to about 150 mg of paeonol.

In certain aspects, the compositions of the present disclosure comprise about 15 mg of CBD. In certain aspects, the compositions of the present disclosure comprise 15 mg of CBD. In certain aspects, the compositions of the present disclosure comprise about 20 mg of CBD. In certain aspects, the compositions of the present disclosure comprise 20 mg of CBD. In certain aspects, the compositions of the present disclosure comprise about 25 mg of CBD. In certain aspects, the compositions of the present disclosure comprise 25 mg of CBD. In certain aspects, the compositions of the present disclosure comprise about 30 mg of CBD. In certain aspects, the compositions of the present disclosure comprise 30 mg of CBD. In certain aspects, the compositions of the present disclosure comprise about 40 mg of CBD. In certain aspects, the compositions of the present disclosure comprise 40 mg of CBD. In certain aspects, the compositions of the present disclosure comprise about 50 mg of CBD. In certain aspects, the compositions of the present disclosure comprise 50 mg of CBD.

In certain aspects, the compositions of the present disclosure comprise about 1 mg of CBN. In certain aspects, the compositions of the present disclosure comprise 1 mg of CBN. In certain aspects, the compositions of the present disclosure comprise about 2 mg of CBN. In certain aspects, the compositions of the present disclosure comprise 2 mg of CBN. In certain aspects, the compositions of the present disclosure comprise about 2.5 mg of CBN. In certain aspects, the compositions of the present disclosure comprise 2.5 mg of CBN. In certain aspects, the compositions of the present disclosure comprise about 4 mg of CBN. In certain aspects, the compositions of the present disclosure comprise 4 mg of CBN. In certain aspects, the compositions of the present disclosure comprise about 5 mg of CBN. In certain aspects, the compositions of the present disclosure comprise 5 mg of CBN. In certain aspects, the compositions of the present disclosure comprise about 10 mg of CBN. In certain aspects, the compositions of the present disclosure comprise 10 mg of CBN. In certain aspects, the compositions of the present disclosure comprise about 20 mg of CBN. In certain aspects, the compositions of the present disclosure comprise 20 mg of CBN. In certain aspects, the compositions of the present disclosure comprise about 40 mg of CBN. In certain aspects, the compositions of the present disclosure comprise 40 mg of CBN.

In certain aspects, the compositions of the present disclosure comprise cannabigerol (CBG) in the amount of 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.1 mg, about 2.2 mg, about 2.3 mg, about 2.4 mg, about 2.5 mg, about 2.6 mg, about 2.7 mg, about 2.8 mg, about 2.9 mg, about 3 mg, about 3.1 mg, about 3.2 mg, about 3.3 mg, about 3.4 mg, about 3.5 mg, about 3.6 mg, about 3.7 mg, about 3.8 mg, about 3.9 mg, about 4 mg, about 4.1 mg, about 4.2 mg, about 4.3 mg, about 4.4 mg, about 4.5 mg, about 4.6 mg, about 4.7 mg, about 4.8 mg, about 4.9 mg, about 5 mg, about 5.1 mg, about 5.2 mg, about 5.3 mg, about 5.4 mg, about 5.5 mg, about 5.6 mg, about 5.7 mg, about 5.8 mg, about 5.9 mg, about 6 mg, about 6.1 mg, about 6.2 mg, about 6.3 mg, about 6.4 mg, about 6.5 mg, about 6.6 mg, about 6.7 mg, about 6.8 mg, about 6.9 mg, about 7 mg, about 7.1 mg, about 7.2 mg, about 7.3 mg, about 7.4 mg, about 7.5 mg, about 7.6 mg, about 7.7 mg, about 7.8 mg, about 7.9 mg, about 8 mg, about 8.1 mg, about 8.2 mg, about 8.3 mg, about 8.4 mg, about 8.5 mg, about 8.6 mg, about 8.7 mg, about 8.8 mg, about 8.9 mg, about 9 mg, about 9.1 mg, about 9.2 mg, about 9.3 mg, about 9.4 mg, about 9.5 mg, about 9.6 mg, about 9.7 mg, about 9.8 mg, about 9.9 mg, about 10 mg, about 10.1 mg, about 10.2 mg, about 10.3 mg, about 10.4 mg, about 10.5 mg, about 10.6 mg, about 10.7 mg, about 10.8 mg, about 10.9 mg, about 11 mg, about 11.1 mg, about 11.2 mg, about 11.3 mg, about 11.4 mg, about 11.5 mg, about 11.6 mg, about 11.7 mg, about 11.8 mg, about 11.9 mg, about 12 mg, about 12.1 mg, about 12.2 mg, about 12.3 mg, about 12.4 mg, about 12.5 mg, about 12.6 mg, about 12.7 mg, about 12.8 mg, about 12.9 mg, about 13 mg, about 13.1 mg, about 13.2 mg, about 13.3 mg, about 13.4 mg, about 13.5 mg, about 13.6 mg, about 13.7 mg, about 13.8 mg, about 13.9 mg, about 14 mg, about 14.1 mg, about 14.2 mg, about 14.3 mg, about 14.4 mg, about 14.5 mg, about 14.6 mg, about 14.7 mg, about 14.8 mg, about 14.9 mg, about 15 mg, about 20 mg, about 25 mg, or about 30 mg. In certain aspects, the compositions of the present disclosure comprise about 7.5 mg of CBG. In certain aspects, the compositions of the present disclosure comprise 7.5 mg of CBG. In certain aspects, the compositions of the present disclosure comprise about 10 mg of CBG. In certain aspects, the compositions of the present disclosure comprise 10 mg of CBG. In certain aspects, the compositions of the present disclosure comprise about 15 mg of CBG. In certain aspects, the compositions of the present disclosure comprise 15 mg of CBG. In certain aspects, the compositions of the present disclosure comprise about 20 mg of CBG. In certain aspects, the compositions of the present disclosure comprise 20 mg of CBG.

In certain aspects, the compositions of the present disclosure comprise 3 mg of Piper nigrum. In certain aspects, the compositions of the present disclosure comprise about 6 mg of Piper nigrum. In certain aspects, the compositions of the present disclosure comprise 6 mg of Piper nigrum.

In certain aspects, the compositions of the present disclosure comprise about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg of Magnolia officinalis.

In certain aspects, the compositions of the present disclosure comprise about 10 mg, about 20 mg, about 30 mg, about 40 mg, about 50 mg, about 60 mg, about 70 mg, about 80 mg, about 90 mg, or about 100 mg of Piper methysticum,

In certain aspects, the compositions of the present disclosure comprise one or more essential oils from Citrus species. In some aspects, the Citrus species is Citrus reticulata (e.g., Citrus reticulate Blanco essential oil). In certain aspects, the compositions of the present disclosure comprise Citrus reticulata (e.g., Citrus reticulata Blanco essential oil) in the amount of about 5 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, or about 150 mg. In certain aspects, the compositions of the present disclosure comprise about 50 mg of Citrus reticulata (e.g., Citrus reticulata Blanco essential oil). In certain aspects, the compositions of the present disclosure comprise 50 mg of Citrus reticulata (e.g., Citrus reticulata Blanco essential oil). In certain aspects, the compositions of the present disclosure comprise about 100 mg of Citrus reticulata (e.g., Citrus reticulata Blanco essential oil). In certain aspects, the compositions of the present disclosure comprise 100 mg of Citrus reticulata (e.g., Citrus reticulata Blanco essential oil).

In certain aspects, the compositions of the present disclosure comprise about 80 mg of Lavendula officinalis essential oil. In certain aspects, the compositions of the present disclosure comprise 80 mg of Lavendula officinalis essential oil. In certain aspects, the compositions of the present disclosure comprise about 160 mg of Lavendula officinalis essential oil. In certain aspects, the compositions of the present disclosure comprise 160 mg of Lavendula officinalis essential oil.

In certain aspects, the compositions of the present disclosure comprise about 90 mg of Polygala tenuifolia. In certain aspects, the compositions of the present disclosure comprise 90 mg of Polygala tenuifolia. In certain aspects, the compositions of the present disclosure comprise about 180 mg of Polygala tenuifolia. In certain aspects, the compositions of the present disclosure comprise 180 mg of Polygala tenuifolia.

In certain aspects, the compositions of the present disclosure comprise about 125 mg of Withania somnifera. In certain aspects, the compositions of the present disclosure comprise 125 mg of Withania somnifera. In certain aspects, the compositions of the present disclosure comprise about 200 mg of Withania somnifera. In certain aspects, the compositions of the present disclosure comprise 200 mg of Withania somnifera. In certain aspects, the compositions of the present disclosure comprise about 250 mg of Withania somnifera. In certain aspects, the compositions of the present disclosure comprise 250 mg of Withania somnifera. In certain aspects, the compositions of the present disclosure comprise about 400 mg of Withania somnifera. In certain aspects, the compositions of the present disclosure comprise 400 mg of Withania somnifera.

In certain aspects, the compositions of the present disclosure comprise rutaecarpine in the amount of about 30 mg, about 40 mg, about 50 mg, about 60 mg, about 70 mg, about 80 mg, about 90 mg, about 100 mg, about 110 mg, about 120 mg, about 130 mg, about 140 mg, or about 150 mg. In certain aspects, the compositions of the present disclosure comprise about 50 mg of rutaecarpine. In certain aspects, the compositions of the present disclosure comprise 50 mg of rutaecarpine. In certain aspects, the compositions of the present disclosure comprise about 100 mg of rutaecarpine. In certain aspects, the compositions of the present disclosure comprise 100 mg of rutaecarpine.

Rutaecarpine is an indolopyridoquinazolinone alkaloid isolated from Evodia rutaecarpa and related herbs, which has shown a variety of biological properties such as anti-thrombotic, anticancer, anti-inflammatory and analgesic, anti-obesity and thermoregulatory, vasorelaxing activity, as well as effects on the cardiovascular and endocrine systems, and upregulation of the metabolism of caffeine. See e.g., Lee S H. et al., Molecules., 13(2):272-300 (2008).

In certain aspects, the compositions of the present disclosure comprise about 50 mg of Passiflora incarnata. In certain aspects, the compositions of the present disclosure comprise 50 mg of Passiflora incarnata. In certain aspects, the compositions of the present disclosure comprise about 100 mg of Passiflora incarnata. In certain aspects, the compositions of the present disclosure comprise 100 mg of Passiflora incarnata.

In certain aspects, the compositions of the present disclosure comprise Ganoderma lucidum in the amount of about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg. In certain aspects, the compositions of the present disclosure comprise about 150 mg of Ganoderma lucidum. In certain aspects, the compositions of the present disclosure comprise 150 mg of Ganoderma lucidum. In certain aspects, the compositions of the present disclosure comprise about 175 mg of Ganoderma lucidum. In certain aspects, the compositions of the present disclosure comprise 175 mg of Ganoderma lucidum. In certain aspects, the compositions of the present disclosure comprise about 300 mg of Ganoderma lucidum. In certain aspects, the compositions of the present disclosure comprise 300 mg of Ganoderma lucidum. In certain aspects, the compositions of the present disclosure comprise about 350 mg of Ganoderma lucidum. In certain aspects, the compositions of the present disclosure comprise 350 mg of Ganoderma lucidum.

Ganoderma is a genus of about 80 species of polypore fungi in the family Ganodermataceae. Ganoderma species of the present disclosure include, but are not limited to, Ganoderma lucidum (reishi, lingzhi; see e.g., Wachtel-Galor S., et al., Ganoderma lucidum (Lingzhi or Reishi): A Medicinal Mushroom. In: Benzie I F F, Wachtel-Galor S, editors. Herbal Medicine: Biomolecular and Clinical Aspects. 2^{nd} edition. Boca Raton (FL): CRC Press/Taylor & Francis; 2011. Chapter 9)), Ganoderma applanatum (artist's conk), Ganoderma multipileum, and Ganoderma tsugae (hemlock varnish shelf).

In certain aspects, the compositions of the present disclosure comprise about 100 mg of Matricaria recutita. In certain aspects, the compositions of the present disclosure comprise 100 mg of Matricaria recutita. In certain aspects, the compositions of the present disclosure comprise about 200 mg of Matricaria recutita. In certain aspects, the compositions of the present disclosure comprise 200 mg of Matricaria recutita.

In certain aspects, the compositions of the present disclosure comprise about 30 mg of Pueraria lobata. In certain aspects, the compositions of the present disclosure comprise 30 mg of Pueraria lobata. In certain aspects, the compositions of the present disclosure comprise about 50 mg of Pueraria lobata. In certain aspects, the compositions of the present disclosure comprise 50 mg of Pueraria lobata. In certain aspects, the compositions of the present disclosure comprise about 60 mg of Pueraria lobata. In certain aspects, the compositions of the present disclosure comprise 60 mg of Pueraria lobata. In certain aspects, the compositions of the present disclosure comprise about 100 mg of Pueraria lobata. In certain aspects, the compositions of the present disclosure comprise 100 mg of Pueraria lobata.

In certain aspects, the compositions of the present disclosure comprise about 125 mg of Ziziphus spinosa. In certain aspects, the compositions of the present disclosure comprise 125 mg of Ziziphus spinosa. In certain aspects, the compositions of the present disclosure comprise about 250 mg of Ziziphus spinosa. In certain aspects, the compositions of the present disclosure comprise 250 mg of Ziziphus spinosa.

In certain aspects, the compositions of the present disclosure comprise about 25 mg of Sceletium tortuosum. In certain aspects, the compositions of the present disclosure comprise 25 mg of Sceletium tortuosum. In certain aspects, the compositions of the present disclosure comprise about 50 mg of Sceletium tortuosum. In certain aspects, the compositions of the present disclosure comprise 50 mg of Sceletium tortuosum.

In certain aspects, the compositions of the present disclosure comprise Crocus sativus in the amount of about 5 mg, about 10 mg, about 15 mg, about 20 mg, about 25 mg, about 30 mg, about 35 mg, about 40 mg, about 45 mg, or about 50 mg. In certain aspects, the compositions of the present disclosure comprise about 15 mg of Crocus sativus. In certain aspects, the compositions of the present disclosure comprise 15 mg of Crocus sativus. In certain aspects, the compositions of the present disclosure comprise about 30 mg of Crocus sativus. In certain aspects, the compositions of the present disclosure comprise 30 mg of Crocus sativus.

Crocus is a genus of about 90 species of flowering plants in the family Iridaceae. Crocus species of the present disclosure include, but are not limited to, Crocus sativus (saffron crocus; see e.g., Khazdair M R et al., Avicenna J Phytomed., 5(5):376-391 (2015)), Crocus asumaniae, Crocus cartwrightianus, Crocus hadriaticus, Crocus pallasii, and Crocus thomasii.

In certain aspects, the compositions of the present disclosure comprise about 150 mg of Schisandra chinensis. In certain aspects, the compositions of the present disclosure comprise 150 mg of Schisandra chinensis. In certain aspects, the compositions of the present disclosure comprise about 300 mg of Schisandra chinensis. In certain aspects, the compositions of the present disclosure comprise 300 mg of Schisandra chinensis.

In certain aspects, the compositions of the present disclosure comprise dihydromyricetin in the amount of about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg. In certain aspects, the compositions of the present disclosure comprise about 125 mg of dihydromyricetin. In certain aspects, the compositions of the present disclosure comprise 125 mg of dihydromyricetin. In certain aspects, the compositions of the present disclosure comprise about 250 mg of dihydromyricetin. In certain aspects, the compositions of the present disclosure comprise 250 mg of dihydromyricetin.

Dihydromyricetin is a flavonoid isolated from Ampelopsis grossedentata, which is traditionally used in China. Dihydromyricetin exhibits health-benefiting activities with minimum adverse effects. Dihydromyricetin has been demonstrated to show antioxidative, anti-inflammatory, anticancer, antimicrobial, cell death-mediating, lipid and glucose metabolism-regulatory activities, as well as detoxicant and anxiolytic properties. See e.g., Li H. et al., Evid Based Complement Alternat Med., 2017:1053617 (2017); Shen Y, et al. J Neurosci, 32(1): 390-401 (2012).

In certain aspects, the compositions of the present disclosure comprise about 25 mg of 5-HTP. In certain aspects, the compositions of the present disclosure comprise 25 mg of 5-HTP. In certain aspects, the compositions of the present disclosure comprise about 50 mg of 5-HTP. In certain aspects, the compositions of the present disclosure comprise 50 mg of 5-HTP.

In certain aspects, the compositions of the present disclosure comprise about 125 mg of Poria cocos. In certain aspects, the compositions of the present disclosure comprise 125 mg of Poria cocos. In certain aspects, the compositions of the present disclosure comprise about 250 mg of Poria cocos. In certain aspects, the compositions of the present disclosure comprise 250 mg of Poria cocos.

In certain aspects, the compositions of the present disclosure comprise about 50 mg of Panax ginseng. In certain aspects, the compositions of the present disclosure comprise 50 mg of Panax ginseng. In certain aspects, the compositions of the present disclosure comprise about 75 mg of Panax ginseng. In certain aspects, the compositions of the present disclosure comprise 75 mg of Panax ginseng. In certain aspects, the compositions of the present disclosure comprise about 100 mg of Panax ginseng. In certain aspects, the compositions of the present disclosure comprise 100 mg of Panax ginseng. In certain aspects, the compositions of the present disclosure comprise about 150 mg of Panax ginseng. In certain aspects, the compositions of the present disclosure comprise 150 mg of Panax ginseng.

In certain aspects, the compositions of the present disclosure comprise Centella asiatica in the amount of about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg. In certain aspects, the compositions of the present disclosure comprise about 165 mg of Centella asiatica. In certain aspects, the compositions of the present disclosure comprise 165 mg of Centella asiatica. In certain aspects, the compositions of the present disclosure comprise about 330 mg of Centella asiatica. In certain aspects, the compositions of the present disclosure comprise 330 mg of Centella asiatica.

Centella is a genus of about 53 species of flowering plants in the subfamily Mackinlayoideae. Centella species of the present disclosure include, but are not limited to, Centella asiatica (see e.g., Gohil K J et al., Indian J Pharm Sci., 72(5):546-556 (2010), Centella abbreviate, Centella brachycarpa, Centella caespitosa, Centella calcaria, Centella callioda, Centella capensis, Centella cochlearia, Centella comptonii, Centella cordifolia, Centella coriacea, Centella restioides, Centella rupestris, Centella scabra, Centella sessilis, Centella stenophylla, Centella stipitata, Centella ternata, Centella thesioides, Centella tridentate, Centella umbellate, Centella villosa, and Centella virgata.

In certain aspects, the compositions of the present disclosure comprise about 50 mg of paeonol. In certain aspects, the compositions of the present disclosure comprise 50 mg of paeonol. In certain aspects, the compositions of the present disclosure comprise about 100 mg of paeonol. In certain aspects, the compositions of the present disclosure comprise 100 mg of paeonol.

In certain aspects, the compositions of the present disclosure comprise about 20 mg of cannabidiol (CBD), about 1 mg of cannabinol (CBN), about 3 mg of Piper nigrum, about 30 mg Lavandula officinalis, about 30 mg of Piper methysticum, and about 125 mg of Withania somnifera.

In certain aspects, the compositions of the present disclosure comprise about 40 mg of cannabidiol (CBD), about 2 mg of cannabinol (CBN), about 6 mg of Piper nigrum, about 60 mg Lavandula officinalis, about 60 mg of Piper methysticum, and about 250 mg of Withania somnifera.

In certain aspects, the compositions of the present disclosure comprise about 25 mg of cannabidiol (CBD), about 2.5 mg of cannabinol (CBN), about 3 mg of Piper nigrum, about 50 mg rutaecarpine, about 40 mg of Passiflora incarnata, about 20 mg of Magnolia officinalis, and about 175 mg of Ganoderma lucidum.

In certain aspects, the compositions of the present disclosure comprise about 50 mg of cannabidiol (CBD), about 5 mg of cannabinol (CBN), about 6 mg of Piper nigrum, about 100 mg rutaecarpine, about 80 mg of Passiflora incarnata, about 40 mg of Magnolia officinalis, and about 350 mg of Ganoderma lucidum.

In certain aspects, the compositions of the present disclosure comprise about 20 mg of cannabidiol (CBD), about 2 mg of cannabinol (CBN), about 5 mg of cannabigerol (CBG), about 100 mg of Matricaria recutita, about 30 mg of Pueraria lobata, about 75 mg of Ziziphus spinosa, about 8 mg of Sceletium tortuosum, and about 25 mg of Citrus reticulata (e.g., Citrus reticulata Blanco essential oil).

In certain aspects, the compositions of the present disclosure comprise about 40 mg of cannabidiol (CBD), about 4 mg of cannabinol (CBN), about 10 mg of cannabigerol (CBG), about 200 mg of Matricaria recutita, about 60 mg of Pueraria lobata, about 150 mg of Ziziphus spinosa, about 16 mg of Sceletium tortuosum, and about 50 mg of Citrus reticulata (e.g., Citrus reticulata Blanco essential oil).

In certain aspects, the compositions of the present disclosure comprise about 20 mg of cannabidiol (CBD), about 4 mg of cannabigerol (CBG), about 200 mg of Withania somnifera, about 15 mg of Crocus sativus, about 25 mg of Polygala tenuifolia, and about 25 mg of limonene.

In certain aspects, the compositions of the present disclosure comprise about 40 mg of cannabidiol (CBD), about 8 mg of cannabigerol (CBG), about 400 mg of Withania somnifera, about 30 mg of Crocus sativus, about 50 mg of Polygala tenuifolia, and about 100 mg of Citrus reticulata (e.g., Citrus reticulata Blanco essential oil).

In certain aspects, the compositions of the present disclosure comprise about 25 mg of cannabidiol (CBD), about 5 mg of cannabigerol (CBG), about 3 mg of Piper nigrum, about 150 mg of Schisandra chinensis, and about 125 mg of dihydromyricetin.

In certain aspects, the compositions of the present disclosure comprise about 50 mg of cannabidiol (CBD), about 10 mg of cannabigerol (CBG), about 6 mg of Piper nigrum, about 300 mg of Schisandra chinensis, and about 250 mg of dihydromyricetin.

In certain aspects, the compositions of the present disclosure comprise about 25 mg of cannabidiol (CBD), about 5 mg of cannabinol (CBN), about 3 mg of Piper nigrum, about 25 mg of 5-HTP, about 125 mg of Ziziphus spinosa, and about 125 mg of Poria cocos.

In certain aspects, the compositions of the present disclosure comprise about 50 mg of cannabidiol (CBD), about 10 mg of cannabinol (CBN), about 6 mg of Piper nigrum, about 50 mg of 5-HTP, about 250 mg of Ziziphus spinosa, and about 250 mg of Poria cocos.

In certain aspects, the compositions of the present disclosure comprise about 25 mg of cannabidiol (CBD), about 2.5 mg of cannabinol (CBN), about 5 mg of cannabigerol (CBG), about 3 mg of Piper nigrum, about 50 mg of Panax ginseng, about 50 mg of Pueraria lobata, and about 165 mg of Centella asiatica.

In certain aspects, the compositions of the present disclosure comprise about 50 mg of cannabidiol (CBD), about 5 mg of cannabinol (CBN), about 10 mg of cannabigerol (CBG), about 6 mg of Piper nigrum, about 100 mg of Panax ginseng, about 100 mg of Pueraria lobata, and about 330 mg of Centella asiatica.

In certain aspects, the compositions of the present disclosure comprise about 25 mg of cannabidiol (CBD), about 2.5 mg of cannabinol (CBN), about 5 mg of cannabigerol (CBG), about 3 mg of Piper nigrum, about 75 mg of Panax ginseng, about 150 mg of Ganoderma lucidum, and about 50 mg of paeonol.

In certain aspects, the compositions of the present disclosure comprise about 50 mg of cannabidiol (CBD), about 5 mg of cannabinol (CBN), about 10 mg of cannabigerol (CBG), about 6 mg of Piper nigrum, about 150 mg of Panax ginseng, about 300 mg of Ganoderma lucidum, and about 100 mg of paeonol.

In some aspects, the compositions of the present disclosure further comprise about 5 mg to about 175 mg of medium-chain triglyceride (MCT) oil, about 5 mg to about 100 mg of beeswax (e.g., yellow USP), about 1 mg to about 30 mg sunflower lecithin, about 1 mg to about 30 mg glycerin (e.g., 99.6%, USP, vegetable-based), or any combination thereof.

In some aspects, the compositions of the present disclosure further comprise about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, or about 175 mg of medium-chain triglyceride (MCT) oil.

In some aspects, the compositions of the present disclosure further comprise about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, or about 100 mg of beeswax (e.g., yellow USP).

In some aspects, the compositions of the present disclosure further comprise about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, or about 30 mg of sunflower lecithin.

In some aspects, the compositions of the present disclosure further comprise about 0 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, or about 30 mg of glycerin (e.g., 99.6%, USP, vegetable-based).

### III. Methods of Making Compositions

Also provided herein are methods of making the compositions of the disclosure. In some aspects, the methods comprise using a novel extraction technique to facilitate the required media for incorporating active phytochemicals (chemicals produced by plants) into a slurry that contains pharmacologically active constituents/pharmacophores.

In some aspects, the methods of making the compositions of the disclosure comprise using concentrated (e.g., 4:1 Lycopus spp.) extract ingredients with the crude herb (dried plant material) to achieve a broad spectrum activity, as well as significant levels of pharmacological documented lipophilic active constituents from botanicals.

In some aspects, the concentrated extract ingredients and the crude herbs are added to medium chain triglycerides (MCT) for extraction. In some aspects, this oil maceration is then heated to 105° F., stirred vigorously throughout the day, and separated from the marc (solid residue) at 24 hr. In some aspects, the marc is pressed and the resulting expelled oil is then added back to the recovered MCT menstruum (solvent), which provides a broad spectrum pharmacologically active lipophilic vehicle for the softgel slurry.

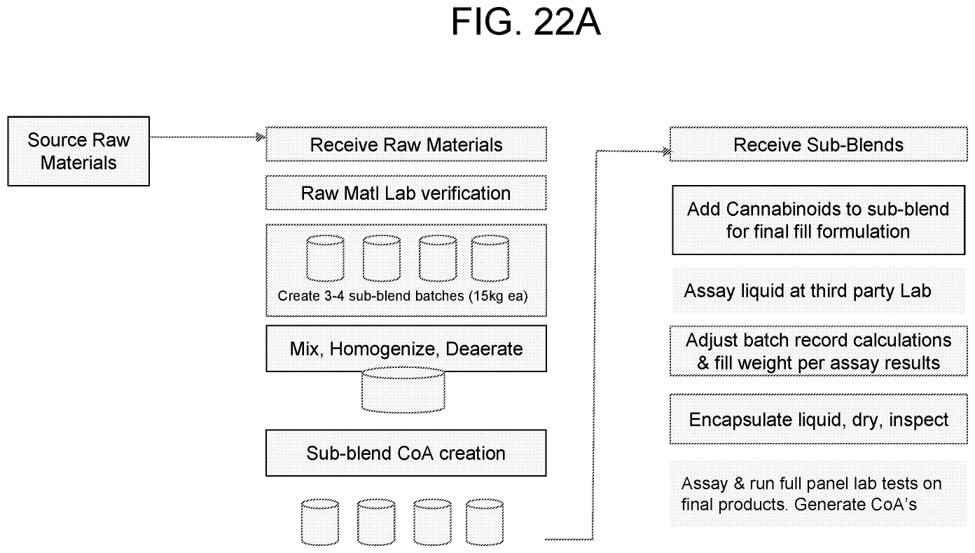

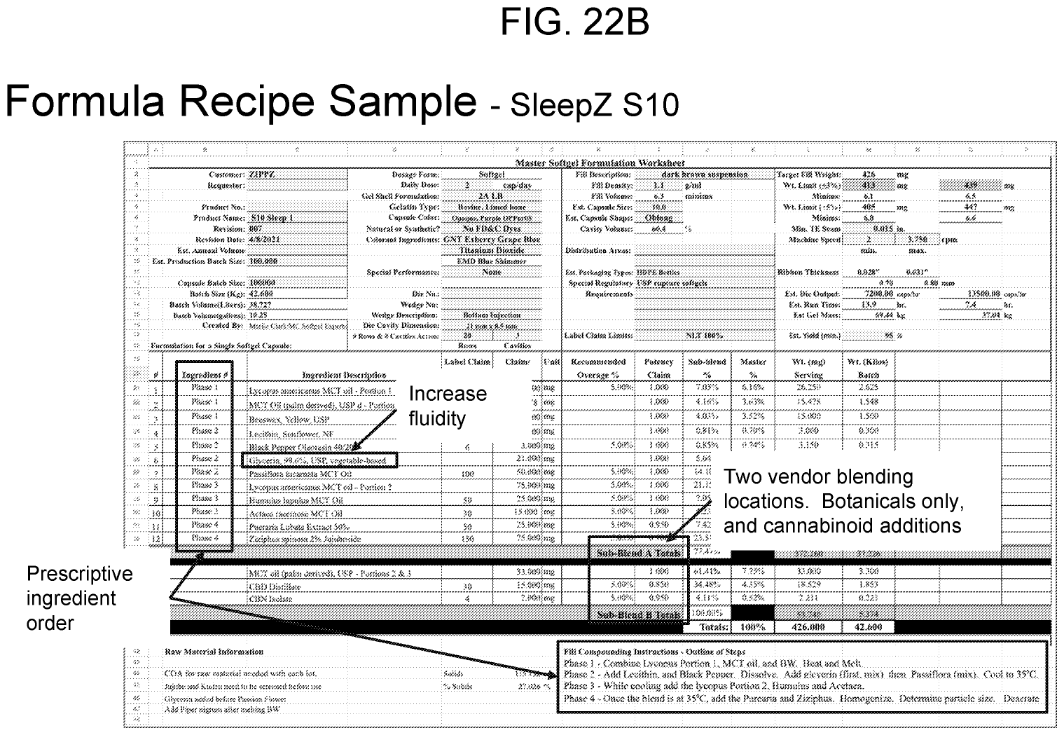

The manufacturing process for the compositions of the disclosure break down into two manufacturing specialties, 1) sub-blend manufacturing—which is the non-cannabinoid ingredient handling, processing, and blending, and 2) cannabinoid handling and final blend creation for softgel encapsulation. See e.g., FIG. 22A.

To ensure consistency between manufacturing runs, ingredient-sourcing goes through a material identification and verification process, assuring assays and material subcomponent details are as intended. Once raw materials are confirmed, the raw sub-blend for each formula is made based on specific quantities, mixing phases, and handling guidelines as shown in the predetermined formula recipes (see e.g., example S10 recipe in FIG. 22B). Materials that require sifting, or particle breakdown, pre-heating, cooling, or other pre-blend manufacturing preparation are completed for 3-4 smaller sub-blend batches, to ensure appropriate consistency across the replicated process. Materials are also sub-divided for phased inclusion based on, for example, the expected fluidity of the batch at each step, or when there could be a material reaction that needs to be controlled by order inclusion. Once the necessary small sub-blend batches are complete per formula, they are combined for large scale mixing with focus on reaching homogeneity and deaeration. After the appropriate period of mixing, the sub-blend is broken back down into sub-batches for use in the next stage.

The appropriate quantities of cannabinoids and additional MCT required per formula are then added to the raw sub-blend. Amounts are calculated based on prior cannabinoid assay values to ensure appropriate label claim potencies. Once cannabinoids are added, the formulation is verified with the liquid full sub-blend format to ensure cannabinoid ingredient assays are within required specification within the full ingredient matrix. At this point, adjustments can be made prior to encapsulation if needed.

With liquid active assay values in range, the process to continue with encapsulation is followed as laid out in the master batch record. Based on the final liquid assay potencies, the batch record calculates the capsule fill weight target, ensuring individual capsules meet that formula's active ingredient label claim. After capsule encapsulation and appropriate drying are complete, a sample of final product capsules undergo final product assay and full panel lab testing, which become the product's published Certificate of Analysis (CoA). After final visual inspections of the capsules, the bulk capsules are bottled and shipped out to a logistics partner for consumer delivery.

### IV. Methods for Improving Sleep, Relieving Pain, and/or Improving Anxiety Symptoms

One aspect of the present disclosure is directed to the methods for improving sleep quality in a subject, comprising administering one or more compositions of the present disclosure.

As described herein, the term “quality of sleep” or “sleep quality” means that the exhausted body and brain can come to rest at a sleep. Fatigue cannot be fully relieved by sleeping for a long time without the quality of sleep.

The quality of sleep, as described herein can be evaluated by sleep onset latency, sleep duration, sleep efficiency, sleep dept, or any combination thereof. Examples of indexes measuring the quality of sleep can include the time from the onset of sleep to the appearance of the non-REM sleep (hereinafter referred to as sleep onset latency), the depth of the non-REM sleep in the initial stage of sleep, the ratio of the non-REM sleep time to the whole sleep time, less sleepiness on awakening, the sleep initiation as well as soundness of sleep, dreaming, a satisfaction with the length of sleep time, a fatigue feeling, and/or the depth of the non-REM sleep during the sleep onset latency and in the initial stage of sleep. See e.g., U.S. Patent Appl. Publ. No. 2015/0147418, which is incorporated herein by reference in its entirety.

As used herein, the term “improving the quality of sleep” or “improving sleep quality” means that the quality of sleep described herein is improved or increased as evaluated, for example, by the fact that the ratio of the non-REM sleep time to the whole sleep time is increased, the less sleepiness feeling on awakening is improved, the sleep initiation as well as the soundness of sleep are improved, dreaming is improved (for example, no longer having dreams frequently or a nightmare), a recovery feeling from fatigue is increased (reduction of fatigue feeling), the sleep onset latency is reduced, and/or the non-REM sleep in the initial stage of sleep is deep.

In certain aspects, the improved sleep quality is sleep onset latency. As used herein, sleep onset latency (SOL or sleep latency) is the length of time that it takes to accomplish the transition from full wakefulness to sleep, normally to the lightest of the non-rapid eye movement (REM) sleep stages.

The duration of time between when the lights are turned off (lights out) as the subject attempts to sleep, until the time the subject actually falls asleep, as evidenced by electroencephalographic (EEG) and behavioral parameters changes consistent with sleep, is reported as sleep latency. Sleep latency is the time in minutes from ‘lights out’ that marks the starting of total recording time to the first epoch scored as sleep. See e.g., Shrivastava et al., J Community Hosp Intern MedPerspect., 4(5):24983 (2014), which is incorporated herein by reference in its entirety.

An ideal sleep latency lays the foundation for a solid night's sleep, which occurs in two basic states throughout the night. The two stages of sleep that occur throughout the night are rapid eye movement sleep (REM) and non-rapid eye movement sleep (NREM). REM sleep is a deeper form of sleep than NREM sleep; however, both states of sleep are directly impacted by sleep latency. If an individual has good sleep latency and is able to fall asleep within a reasonable amount of time, they will have a better chance of progressing through the stages of sleep comfortably and in turn, enjoy a deep sleep. See e.g., Shrivastava et al., J Community Hosp Intern Med Perspect., 4(5):24983 (2014).

Sleep latency is related directly to sleep efficiency. See e.g., Reed D L and Sacco W P., J Clin Sleep Med., 12(2):263-266 (2016)), which is incorporated herein by reference in its entirety. If an individual spends the majority of the time that they are in bed in an evening actually asleep, then they are considered to be sleep efficient. However, if an individual spends a lot of the time that they are in bed not actually asleep, but trying to sleep, then that is not considered to be sleep efficient. A sleep efficiency of 85% is considered to be normal, while a sleep efficiency anywhere above 90% is considered to be very good. A sleep efficiency lower than 85% is considered to be poor. See e.g., Reed D L and Sacco W P., J Clin Sleep Med., 12(2):263-266 (2016).

In some aspects, the sleep efficiency is about 85%, about 86%, about 87%, about 88%, about 89%, about 90%, about 91%, abut 92%, about 93%, about 94%, about 96%, about 97%, about 98%, about 99%, or about 100%.

Sleep latency relates directly to sleep efficiency because if a person is able to fall asleep quickly, they are more likely to have an efficient sleep. If a person has ideal sleep latency, they are likely to have good sleep efficiency as well, as the two go hand in hand.

Sleep latency also relates directly to sleep debt, which is the overall effect of not getting enough sleep. Sleep debt can accumulate over time and, in turn, lead to mental and physical fatigue. Sleep debt has a direct impact on sleep latency as those who are extremely tired or have a lot of sleep debt will likely fall asleep quicker than someone who is better rested and does not have any sleep debt. See e.g., Goel N. et al., Semin Neurol., 29(4):320-339 (2009), which is incorporated herein by reference in its entirety.

In some aspects, the improved sleep quality is sleep duration (total sleep time). The sleep duration is the total amount of sleep time from sleep onset to sleep offset and is distributed throughout the sleep time as minutes of Stage N1 sleep, Stage N2 sleep, Stage N3, and rapid eye movement (REM) sleep. See e.g., Shrivastava et al., J Community Hosp Intern Med Perspect., 4(5):24983 (2014). In certain aspects of the present disclosure, the inadequate sleep duration is short sleep (defined as less than 7 hours a night or long sleep (defined as 9 or more hours a night). See e.g., St-Onge et al., Circulation, 134(18):e367-e386 (2016); Cappuccio F. P. et al., Sleep., 33(5):585-592 (2010); Chaput J P, 1 et al., Nat Sci Sleep., 10:421-430 (2018), which are incorporated herein by reference in their entirety. In certain aspects, short sleep duration is less than 7 hours, less than 6 hours, less than 5 hours, less than 4 hours, less than 3 hours, less than 2 hours, less than 1 hour. In certain aspects, the sleep duration is extended.

One aspect of the present disclosure is directed to the methods for relieving pain in a subject, comprising administering one or more compositions of the present disclosure.

As described herein, the term “relieving pain” or “pain relief” means reducing the level of pain experienced by a subject. Pain reduction can be evaluated as described in e.g., Younger et al., Curr Pain Headache Rep., 13(1):39-43 (2009), which is incorporated herein by reference in its entirety.

In some aspects, pain comprises musculoskeletal pain, pain induced by metabolic syndrome, autoimmune pain, chronic pain, diabetic neuropathy pain, polycystic ovary syndrome pain, endometriosis pain, pain induced by cancer, pain induced by inflammation, general pain, pain induced by migraines, pain induced by anxiety, pelvic pain, fibromyalgia pain, pain induced by hypothyroidism, or any combination thereof.

One aspect of the present disclosure is directed to the methods of improving anxiety symptoms comprising administering one or more of the compositions of the present disclosure.

Anxiety is a psychiatric disorder of significant clinical and socioeconomic significance. It is an unpleasant state that involves a complex combination of emotions that include fear, apprehension, and worry. It is often accompanied by physical sensations such as heart palpitations, nausea, chest pain, shortness of breath, or tension headache. Anxiety disorder is a blanket term covering several different forms of abnormal, pathological anxiety, fears, phobias and nervous conditions that can come on suddenly (acute anxiety) and/or gradually over a period of several years (chronic), and can impair or prevent the pursuing of normal daily routines. Anxiety disorders are often debilitating chronic conditions, which can be present from an early age or begin suddenly after a triggering event. They are prone to flare up at times of high stress.

Anxiety is often described as having cognitive, somatic, emotional, and behavioral components. The cognitive component entails expectation of a diffuse and uncertain danger. Somatically the body prepares the organism to deal with threat (known as an emergency reaction): blood pressure and heart rate are increased, sweating is increased, blood flow to the major muscle groups is increased, and immune and digestive system functions are inhibited. Externally, somatic signs of anxiety may include pale skin, sweating, trembling, and pupillary dilation. Emotionally, anxiety causes a sense of dread or panic and physically causes nausea, and chills. Behaviorally, both voluntary and involuntary behaviors can arise directed at escaping or avoiding the source of anxiety. These behaviors are frequent and often maladaptive, being most extreme in anxiety disorders. However, anxiety is not always pathological or maladaptive. It is a common emotion along with fear, anger, sadness, and happiness, and it has a very important function in relation to survival. See e.g., U.S. Pat. No. 8,008,253, which is incorporated herein by reference in its entirety.

In certain aspects, anxiety comprises social anxiety disorder, anxiety due to caffeine excess, generalized anxiety disorder, anxiety in alcoholism, post-traumatic stress disorder, anxiety in the elderly, anxiety induced depression, a specific phobia, obsessive-compulsive disorder, or any combination thereof.

In certain aspects, anxiety symptoms comprise excessive worrying, feeling agitated, restlessness, difficulty concentrating, irritability, tense muscles, trouble falling or staying asleep, panic attacks, irrational fears, avoiding social situations, or any combination thereof.

Any suitable route of administration can be used. For example, any of oral, intraoral, topical, epicutaneous, subcutaneous, transdermal, intramuscular, parenteral, ocular, rectal, vaginal, inhalation, buccal, sublingual and intranasal delivery routes can be suitable.

In certain aspects, the compositions of the present disclosure are formulated for oral delivery. Oral forms of the compositions described herein can be solid or liquid. Suitable dosage forms can be tablets, capsules, pills, granules, suspensions, emulsions, syrups, or solutions.

In certain aspects, the compositions of the present disclosure are packaged in a form for oral administration as discrete units (i.e., solid dosage forms), such as capsules, tablets, sachets, or the like. Preparation of the solid compositions in forms intended for oral administration is within the ability of one skilled in the art. Such compositions can be prepared by methods known in the pharmaceutical formulation art, for example, see Remington's Pharmaceutical Sciences, 18th Ed., (Mack Publishing Company, Easton, Pa., 1990).

In some aspects, the solid dosage form is a capsular dosage form. In certain aspects, the capsular dosage form comprises gelatin material (e.g., softgel). Softgels can be produced in a process as described in e.g., U.S. Pat. No. 8,974,820, which is incorporated herein by reference in its entirety.

In certain aspects, the composition is administered at predetermined time intervals over an extended period of time. In certain aspects, the composition is administered once a day. In certain aspects, the composition is administered once every day. In certain aspects, the composition is administered every other day. In certain aspects, the composition is administered over 1 week, 2 weeks, 1 month, 2 months, 3 months, 6 moths, 1 year, 2 years, 3 years, 4 years, 5 years, 6 years, 7 years, 8 years, 9 years, 10 years, 11 years, or 12-15 years. In certain aspects, the composition is administered only once.

As used herein, combined administration (co-administration) includes simultaneous administration of the compositions in the same or different dosage form, or separate administration of the compositions (e.g., sequential administration). In certain aspects, the compositions of the present disclosure are simultaneously administered (e.g., sleep formulation #2 is administered with sleep formulation #6). In certain aspects, the compositions of the present disclosure are sequentially administered (e.g., sleep formulation #2 can be administered first followed by (e.g., immediately followed by) the administration of sleep formulation #6).

The following examples are offered by way of illustration and not by way of limitation.

### Examples

### Example 1—Cannabinoid Therapy Formulations for Sleep, Pain, and Anxiety Conditions

Exemplary sleep formulations are described in Table 1.

TABLE 1

| | | 1 cap | 2 caps | | | CBD:CBN |
|---|---|---|---|---|---|---|
| SOFTGEL - Sleep formulation S10 | Ingredient | (mg) | (mg) | % | Specifications | Ratio |
| Key parameters: older, women, fall or | CBN | 2 | 4 | 0.81 | Distillate | 7.5 |
| stay asleep, menopause, chronic | CBD | 15 | 30 | 4.83 | Distillate | |
| disease | Passiflora incarnata | 50 | 100 | 16.10 | Liquid Extract | |
| Formulated for those unable to sleep | Ziziphus spinosa | 75 | 150 | 24.15 | pwd* | |
| well due to restlessness, low levels of | (Z. jujuba semen) | | | | | |
| anxiety, acute or chronic diseases. This | Lycopus spp. flos + herba | 100 | 200 | 32.21 | Liquid Extract | |
| formula also promotes improved sleep | Pueraria lobata | 25 | 50 | 8.05 | pwd | |
| for older women suffering symptoms | Actaea racemosa rhizoma | 15 | 30 | 4.83 | Liquid Extract | |
| of menopause such as hot flashes and | Humulus | 25 | 50 | 8.05 | Liquid Extract | |
| night sweats. | Piper nigrum | 3 | 6 | 0.97 | CO2 Ext | |
| | Totals | 310 | 620 | 100.00 | | |
| | | 1 cap | 2 caps | | | CBD:CBN |
| SOFTGEL - Sleep formulation S12 | Ingredient | (mg) | (mg) | % | Specifications | Ratio |
| Key parameters: falling asleep, | CBG | 5 | 10 | 0.83 | Distillate | 3 |
| anxiety, stress | CBD | 15 | 30 | 4.99 | Distillate | |
| Formulated for people who have | Passiflora incarnata herba | 90 | 180 | 29.95 | Liquid Extract | |
| difficulty falling asleep due to stress, | Scutellaria lateriflora herba | 75 | 150 | 24.96 | Liquid Extract | |
| restlessness, or low levels of anxiety. | Lavendula officinalis EO | 40 | 80 | 13.31 | Liquid Extract | |
| | Lycopus spp. herba | 50 | 100 | 16.64 | Liquid Extract | |
| | Piper methysticum | 40 | 80 | 8.32 | Liquid Extract | |
| | Piper nigrum | 3 | 6 | 1.00 | CO2 Ext | |
| | TOTAL | 318 | 636 | 100.00 | | |
| | | 1 cap | 2 caps | | | CBD:CBG |
| SOFTGEL - Sleep formulation S14 | Ingredient | (mg) | (mg) | % | Specifications | Ratio |
| Key parameters: staying asleep, | CBG | 2.5 | 5 | 0.79 | Distillate | 4 |
| depression, anxiety, particularly | CBD | 10 | 20 | 3.16 | Distillate | |
| formulated for women, | Pueraria lobata | 50 | 100 | 15.82 | pwd | |
| but can be used for men; | Matricaria recutita | 100 | 200 | 31.65 | Liquid Extract | |
| Formulated for people who have | Ziziphus spinosa | 75 | 150 | 23.73 | pwd | |
| difficulty staying asleep due to anxiety | (Z. jujuba semen) | | | | | |
| or mild depression. This formula | Magnolia officinalis | 75 | 150 | 23.73 | Liquid Extract | |
| incorporates herbs and supplements to | Melatonin | 0.5 | 1 | 0.16 | Liquid Extract | |
| extend sleep duration. | Piper nigrum | 3 | 6 | 0.95 | CO2 Ext | |
| | TOTAL | 316 | 632 | 100.00 | | |
| | | 1 cap | 2 caps | | | CBD:CBG |
| SOFTGEL - Sleep formulation S16 | Ingredient | (mg) | (mg) | % | Specifications | Ratio |
| Key parameters: staying asleep, | CBG | 1 | 2 | 1.68 | Distillate | 10 |
| depression, anxiety | CBD | 10 | 20 | 3.35 | Distillate | |
| Formulated for those who have | Withania somnifera | 75 | 150 | 25.13 | Liquid Extract | |
| difficulty staying asleep due to anxiety | Matricaria recutita | 50 | 100 | 16.75 | Liquid Extract | |
| or mild depression. This formula | Ziziphus spinosa | 50 | 100 | 16.75 | pwd | |
| incorporates herbs and supplements to | (Z. jujuba semen) | | | | | |
| extend sleep duration. | Polygala tenuifolia | 75 | 150 | 25.13 | Liquid Extract | |
| | Melatonin | 0.25 | 0.5 | 0.17 | pwd | |
| | Citrus reticulata Blanco | 30 | 60 | 10.05 | Citrus reticulata | |
| | | | | | essential oil | |
| | Piper nigrum | 3 | 6 | 1.01 | CO2 Ext | |
| | TOTAL | 294.25 | 586.5 | 100.00 | | |
| | | 1 cap | 2 caps | | | CBD:CBN |
| SOFTGEL - Sleep formulation S22 | Ingredient | (mg) | (mg) | % | Specifications | Ratio |
| Key parameters: sleep apnea, acid | CBN | 5 | 10 | 1.59 | Distillate | 3 |
| reflux, internal body clock problem, | CBD | 15 | 30 | 4.74 | Distillate | |
| obesity, metabolic syndrome (high | EPA/DHA | 50 | 100 | 15.80 | Liquid Extract | |
| blood pressure, high blood sugar, and | Withania sominfera radix | 75 | 150 | 23.70 | Liquid Extract | |
| abnormal cholesterol or triglyceride | Humulus lupulus | 50 | 100 | 15.80 | Liquid Extract | |
| levels) | Cymbopogon flexuosus | 15 | 30 | 4.74 | EO rich in geranila | |
| Formulated for people who need help | essential oil | | | | and neral (citral) | |
| conquering significant sleep disruptors | Citrus reticulata Blanco | 25 | 50 | 7.90 | Liquid Extract | |
| like sleep apnea, digestive problems | Melatonin | 0.5 | 1 | 0.16 | pwd | |
| (e.g., acid reflux), and metabolic | Grindelia | 75 | 150 | 23.70 | Liquid Extract | |
| syndrome challenges. This formula | Mixed tocopherols | 3 | 6 | 0.95 | tocobiol | |
| concentrates on helping with sleep, but | Piper nigrum | 3 | 6 | 0.95 | CO2 Ext | |
| also supports improvement in blood | Totals | 316.5 | 633 | 100.00 | | |
| pressure, high cholesterol, heart | | | | | | |
| disease, digestive needs, and reducing | | | | | | |
| inflammation of the nose and throat | | | | | | |
| area. | | | | | | |
| | | 1 cap | 2 caps | | | CBD:CBN |
| SOFTGEL - Sleep formulation S18 | Ingredient | (mg) | (mg) | % | Specifications | Ratio |
| Key parameters: inverted sleep/wake | CBN | 2 | 4 | 0.64 | Distillate | 7.5 |
| cycles, poor concentration, | CBD | 15 | 30 | 4.80 | Distillate | |
| forgetfulness, malaise, confusion, | Schisandra chinensis | 125 | 250 | 40.03 | Liquid Extract | |
| obesity | Silymarin | 60 | 120 | 19.22 | Liquid Extract | |
| Formulated for those who's sleep/wake | Phosphatidylserine | 50 | 100 | 16.01 | Liquid Extract | |
| cycles are off resulting in brain fog, | R-Alpha lipoic acid | 25 | 50 | 8.01 | Liquid Extract | |
| poor concentration, forgetfulness, and | Anemarrhena asphodeloides | 25 | 50 | 8.01 | Liquid Extract | |
| lack of energy. | rhizoma | | | | | |
| | Melatonin | 1.25 | 2.5 | 0.40 | pwd | |
| | Piper nigrum | 3 | 6 | 0.96 | CO2 Ext | |
| | Totals | 306.25 | 612.5 | 100.00 | | |
| | | 1 cap | 2 caps | | | CBD:CBN |
| SOFTGEL - Sleep formulation S24 | Ingredient | (mg) | (mg) | % | Specifications | Ratio |
| Key parameters: PTSD, high levels of | CBN | 5 | 10 | 1.64 | Distillate | 6 |
| anxiety | CBD | 30 | 60 | 9.82 | Distillate | |
| Formulated for those with, or | 5-HTP | 12.5 | 25 | 4.09 | Liquid Extract | |
| symptoms of PTSD. | Ziziphus spinosa | 100 | 200 | 32.73 | pwd | |
| | (Z. jujuba semen) | | | | | |
| | Poria cocos sclerotium | 75 | 150 | 24.55 | Liquid Extract | |
| | Ligusticum wallichii radix | 55 | 110 | 18.00 | Liquid Extract | |
| | (Ligusticum Chuanxiong) | | | | | |
| | Sceletium | 8 | 16 | 8.18 | standardized ext | |
| | Piper nigrum | 3 | 6 | 0.98 | CO2 Ext | |
| | Totals | 305.5 | 611 | 100.00 | | |
| | | 1 cap | 2 caps | | | CBD:CBN |
| SOFTGEL - Sleep formulation S20 | Ingredient | (mg) | (mg) | % | Specifications | Ratio |
| Key parameters: Older consumer | CBN | 2.5 | 5 | 0.86 | Distillate | 6 |
| Formulated for the older consumer | CBD | 15 | 30 | 5.16 | Distillate | |
| who has trouble sleeping. Targets | Polygala tenuifolia | 75 | 150 | 25.82 | Liquid Extract | |
| general wellness including physical | Melatonin | 1.5 | 3 | 0.52 | pwd | |
| strength, pain and inflammation, sleep | Poria cocos sclerotium | 75 | 150 | 25.82 | Liquid Extract | |
| regulation, and neuroprotection for | Rehmannia glutinosa | 37.5 | 75 | 12.91 | cured wine | |
| improved cognitive function. | | | | | processed pwd | |
| | Panax ginseng | 50 | 100 | 17.21 | Liquid Extract | |
| | Paeonol | 25 | 50 | 8.61 | Liquid Extract | |
| | Piper nigrum | 3 | 6 | 1.03 | CO2 Ext | |
| | TOTAL | 284.5 | 569 | 100.00 | | |

*cap = capsule
*pwd = powder

Table 2 shows an example of how the compositions of the present disclosure (e.g., sleep formulation S10) combine eastern and western philosophies/remedies and botanicals/herbs to target multiple targets on the ECS and other enzymes and receptors that help control sleep, pain, and/or anxiety (e.g., poor sleep due to stress and pre-menopausal symptoms).

TABLE 2

| Primary Receptors | Cannabinoid | Role |
|---|---|---|
| CB1, CB2 | CBD | Homeostasis, Anxiolytic |
| | CBN | Sleep, Inflammation |
| Melatonin, | Hops flower | Restlessness, anxiety |
| α-2 adrenoreceptors + | (Humulus) | |
| MAPK signal pathway | Bugleweed | Menstrual pain |
| | (Lycopus spp.) | Thyroid |
| COX2 | Passionflower | Inflammation |
| | (Passiflora | |
| | incarnate) | |
| Mu opioid | Black Cohosh | Menstrual discomfort |
| | (Actaea racemosa) | |
| Osteoprotegerin + | Pueraria lobata | Bone strength |
| | | Menopause symptoms |
| Adenosine, Dopamine, | Ziziphus spinosa | Sedative |
| Serotonin, GABA | (Z. jujuba semen) | Muscle Strength |
| Broad targets | Black Pepper | Absorption |
| | (Piper nigrum) | Gastrointestinal Issues |
| | | Inflammation |

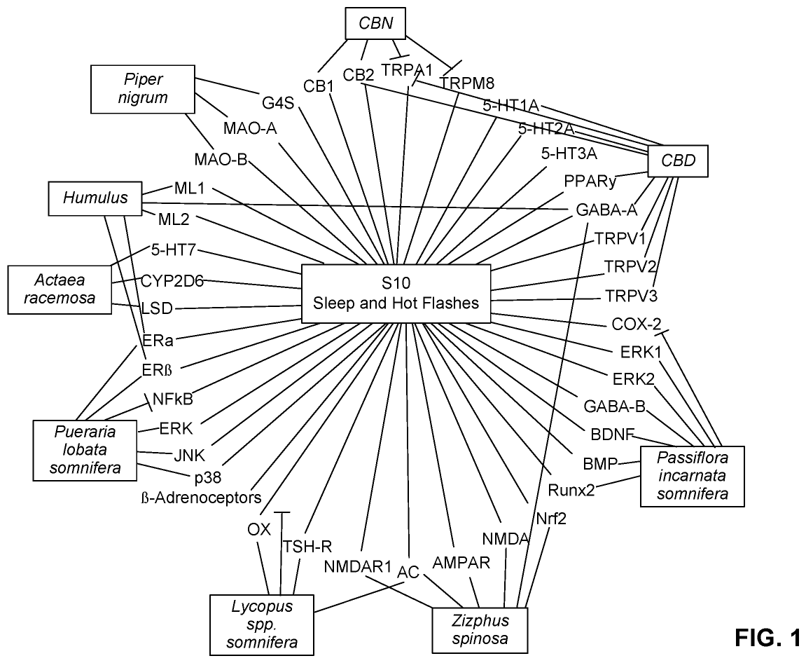

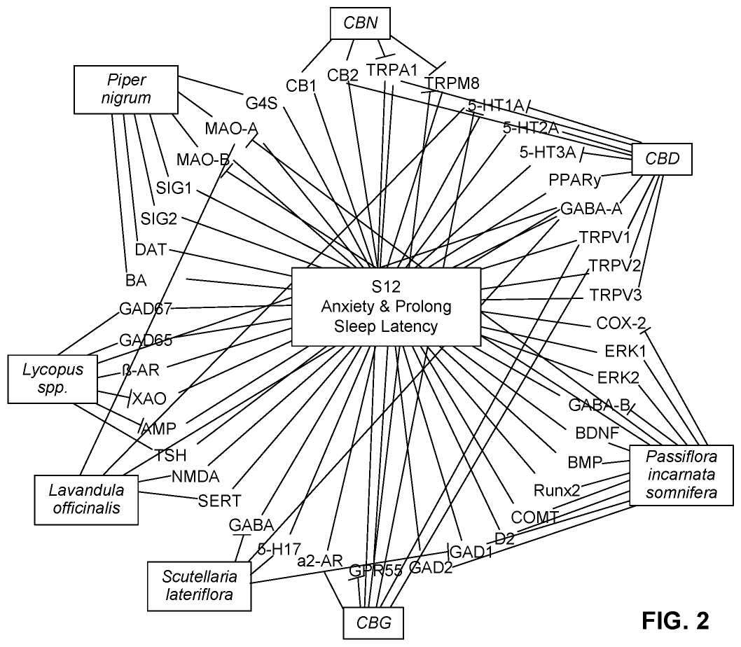

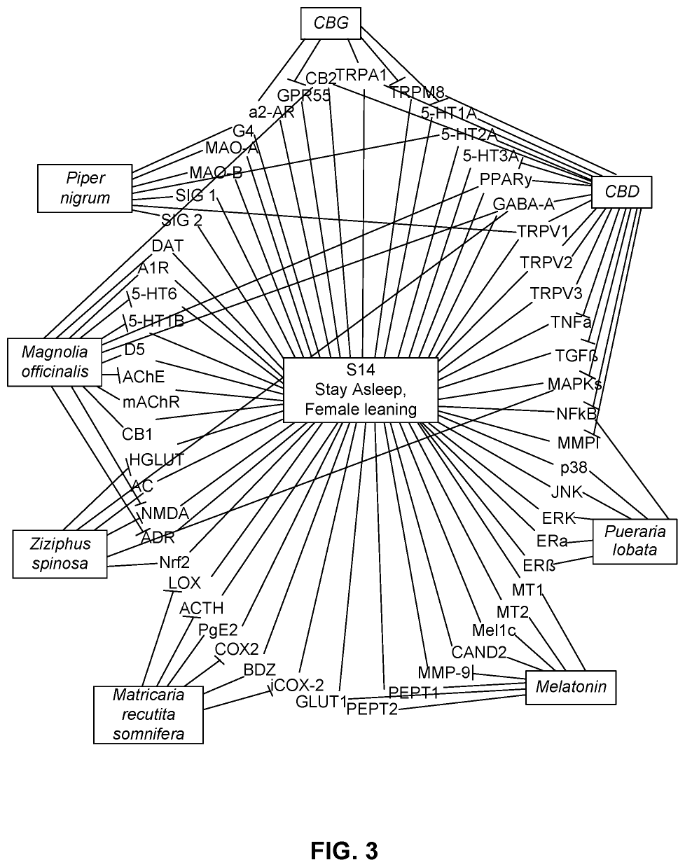

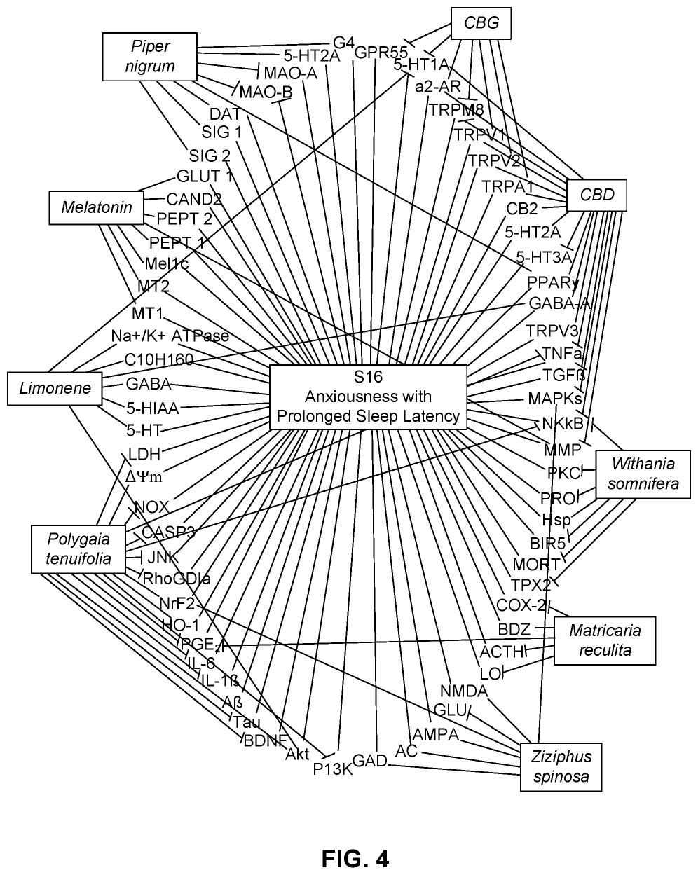

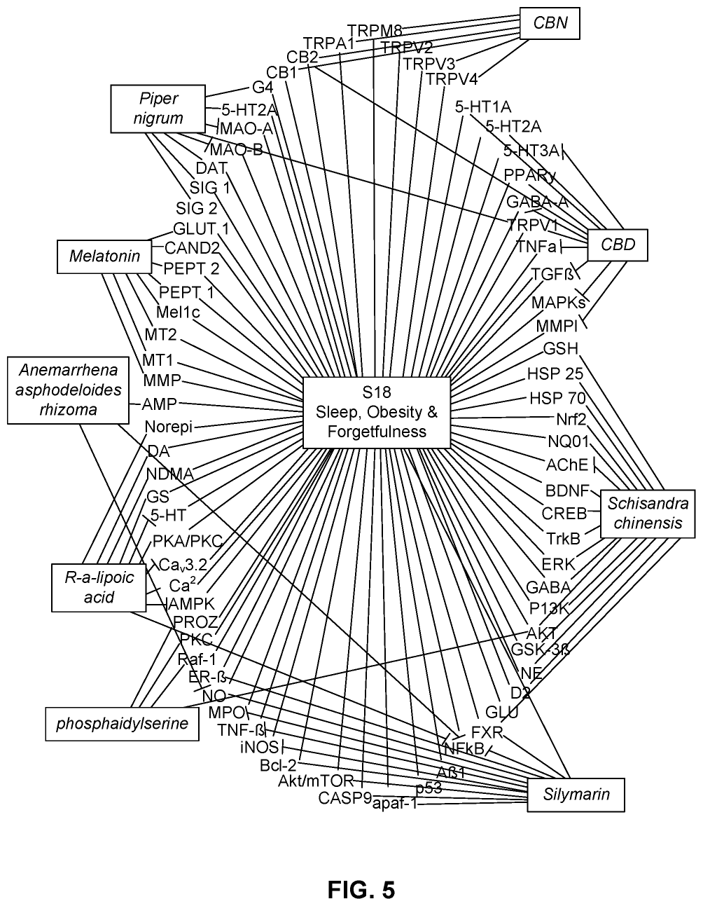

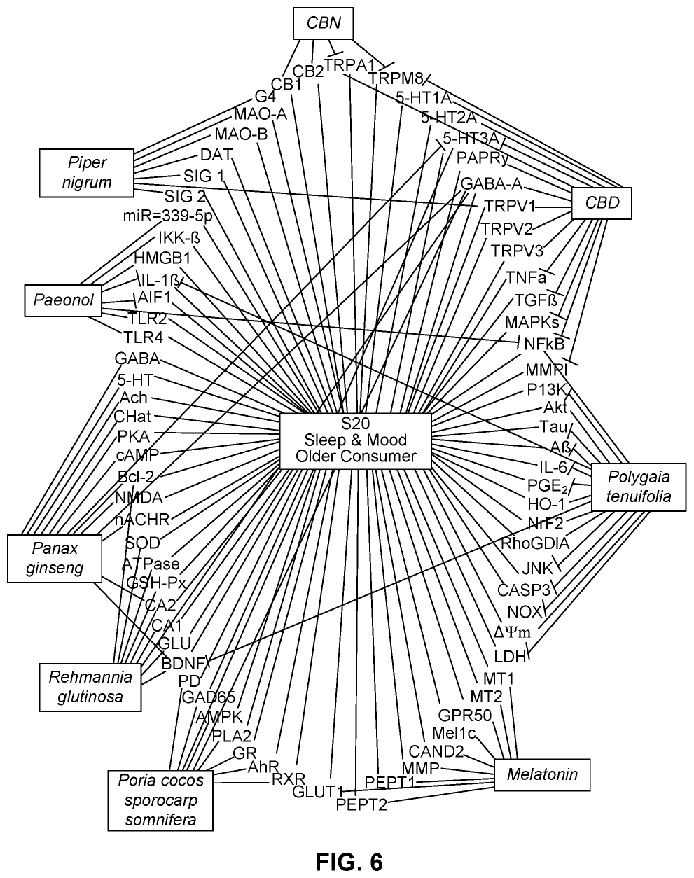

FIGS. 1-6 show examples of how the compositions of the present disclosure (e.g., sleep formulation S10 (FIG. 1), sleep formulation S12 (FIG. 2), sleep formulation S14 (FIG. 3), sleep formulation S16 (FIG. 4), sleep formulation S18 (FIG. 5), and sleep formulation S20 (FIG. 6)) target multiple targets on the ECS and other enzymes and receptors that help control sleep, pain, and/or anxiety.

Exemplary pain formulations are described in Table 3.

TABLE 3

| | | 1 cap | 2 caps | | Delivery | |
|---|---|---|---|---|---|---|
| | Specifications | (mg) | (mg) | % | Method | Actions |
| SOFTGEL formulation P50 | | | | | | |
| Male musculoskeletal pain, metabolic | | | | | | |
| syndrome (MetS) autoimmune pain, | | | | | | |
| chronic pain, diabetic neuropathy | | | | | | |
| CBN | Hemp Distillate | 10.0 | 20 | 3.1 | | Analgesic, anti-inflammatory, anti- |
| | | | | | | nociceptive |
| CBD | Hemp Distillate | 20.0 | 40 | 6.3 | | Anti-inflammatory, anxiolytic, analgesic |
| Fish Oil | EPA:DHA 3:1 must | 65.0 | 130 | 20.3 | softgel | Anti-neuropathic, anti-inflammatory, |
| | be purified and | | | | | antinociceptive, anti-depressant |
| | concentrated | | | | | |
| Curcuma longa | 95% curcumin | 120.0 | 240 | 37.6 | | Anti-inflammatory |
| Nigella oil (fixed) | Standardized to | 65.0 | 130 | 20.3 | | Anti-inflammatory, analgesic, anti- |
| | thymoquinone | | | | | nociceptive, anti-arthritic, |
| | | | | | | hypoglycemic, hypolipidemic |
| Zanthoxylum spp | Made from 95% | 10.0 | 20 | 3.1 | | CB2 agonist, anti-inflammatory, |
| | EtOH extraction | | | | | circulatory stimulant, Bioavailability |
| | | | | | | enhancer for formula, anti-inflammatory |
| Piper nigrum ext | CO2 extract | 3.0 | 6 | 0.9 | | Anesthetic, CB2 agonist, |
| | | | | | | antinociceptive, AEA/2-AG reuptake |
| | | | | | | inhibitor |
| Citrus reticulata (e.g., | minimum 90% | 22.5 | 45 | 7.0 | | Anti-nociceptive, antidepressant, |
| Citrus reticulata Blanco) | limonene | | | | | immune modulating, anti-inflammatory |
| Tocobiol | mixed tocopherols | 4.0 | 8 | 1.3 | | Anti-oxidant, hepatoprotective in |
| | | | | | | NAFLD, decreased Hb1AC |
| Total | | 319.5 | 639 | 100.0 | | |
| SOFTGEL formulation P52 | | | | | | |
| Female polycystic ovary syndrome | | | | | | |
| (PCOS), MetS, endometriosis, | | | | | | |
| musculoskeletal pain, autoimmune | | | | | | |
| pain, chronic pain, diabetic neuropathy | | | | | | |
| CBN | Hemp Distillate | 2.5 | 5 | 0.77 | | Anti-nociceptive, anti-inflammatory |
| CBD | Hemp Distillate | 10.0 | 20 | 3.08 | | Antispasmodic, anxiolytic, analgesic |
| Fish Oil | EPA:DHA 3:1 must | 65.0 | 130 | 20.0 | softgel | Anti-neuropathic, anti-inflammatory, |
| | be purified and | | | | | antinociceptive, anti-depressant, |
| | concentrated | | | | | estrogen attenuating |
| Curcuma longa | 95% curcumin | 120.0 | 240 | 37.0 | | Anti-inflammatory, insulin sensitizing, |
| | | | | | | anti-nociceptive, reduced CX3CR1 |
| Nigella oil (fixed) | Standardized to | 60.0 | 120 | 18.5 | | Anti-inflammatory, analgesic, anti- |
| | thymoquinone | | | | | nociceptive, anti-arthritic, hypoglycemic |
| Zanthoxylum spp | made from 95% | 10.0 | 20 | 3.1 | | CB2 agonist, anti-inflammatory, |
| | EtOH extraction | | | | | circulatory stimulant |
| Citrus reticulata (e.g., | EO minimum 90% | 25.0 | 50 | 7.7 | | Anti-nociceptive, antidepressant, |
| Citrus reticulata Blanco) | limonene | | | | | immune modulating, anti-inflammatory |
| Luteolin | from plant source | 25.0 | 50 | 7.7 | | Protects against diabetic cardiomyopathy, |
| | | | | | | anticancer, anti-inflammatory, reduces |
| | | | | | | neuroinflammation, anti-nociceptive, |
| | | | | | | NFkB inhibition |
| Vitamin D | cholecalciferol | 0.025 | 0.05 | 0.0077 | equivalent | Immunomodulating, beneficial for |
| | 2000 IU | | | | of 2000 IU | metabolic, endocrine and fertility in |
| | | | | | | PCOS |
| Tocobiol | mixed tocopherols | 4.0 | 8 | 1.2 | | Anti-oxidant, hepatoprotective in |
| | | | | | | NAFLD, decreased Hb1AC |
| Piper nigrum | CO2 extract | 3.0 | 6 | 0.9 | | Anesthetic, CB2 agonist, |
| | | | | | | antinociceptive, AEA/2-AG reuptake |
| | | | | | | inhibitor |
| Total | | 324.5 | 649.05 | 100.0 | | |
| SOFTGEL formulation P54 | | | | | | |
| pain induced by cancer, inflammation, | | | | | | |
| general pain relief | | | | | | |
| CBN | Hemp Distillate | 5.0 | 10 | 1.57 | softgel | Anti-nociceptive, anti-inflammatory, |
| | | | | | | anxiolytic |
| CBD | Hemp Distillate | 25.0 | 50 | 7.86 | | Ant-iinflammatory, enhances opiod |
| | | | | | | antinociception, anticancer, anxiolytic |
| Withania somnifera | lyophilized EtOH | 200.0 | 400 | 62.9 | | Stress reduction, adaptogenic, analgesic, |
| | extract | | | | | anti-cancer, antinociceptive, opioidergic |
| Corydalis yanhusuo | 50% THP | 75.0 | 150 | 23.6 | | Antinociceptive, opioidergic |
| Glycyrrhiza glabra | 25% glycyrrhizic | 10.0 | 20 | 3.1 | | Anti-inflammatory, anticancer |
| | acid | | | | | |
| Piper nigrum | CO2 extract | 3.0 | 6 | 0.9 | | Anesthetic, CB2 agonist, |
| | | | | | | antinociceptive, AEA/2-AG reuptake |
| | | | | | | inhibitor |
| Total | | 318.0 | 636 | 100.0 | | |
| SOFTGEL formulation P56 | | | | | | |
| migraines, anxiety | | | | | | |
| CBN | Hemp Distillate | 5.0 | 10 | 1.50 | softgel | Anti-nociceptive, anti-inflammatory, |
| | | | | | | anxiolytic |
| CBD | Hemp Distillate | 15.0 | 30 | 4.50 | | Anti-inflammatory, anxiolytic |
| Lavandula angustifolia | EO: linalool rich CT | 10.0 | 20 | 3.0 | | Anxiolytic, sleep enhancement |
| Zingiber officinalis | Liquid Extract | 75.0 | 150 | 22.5 | | Anti-inflammatory, antinociceptive, |
| | | | | | | anxiolytic, antiflammatory |
| Scutellaria lateriflora | Liquid Extract | 175.0 | 350 | 52.6 | | Anxiolytic, sleep enhancement, anti- |
| | | | | | | migraine |
| CoQ10 | Liquid Form | 50.0 | 100 | 15.0 | | Anti-migraine, antioxidant, pain |
| | | | | | | reduction |
| Piper nigrum | CO2 Extract | 3.0 | 6 | 0.9 | | Anesthetic, CB2 agonist, |
| | | | | | | antinociceptive, AEA/2-AG reuptake |
| | | | | | | inhibitor |
| Total | | 333.0 | 666 | 100.0 | | |
| SOFTGEL formulation P58 | | | | | | |
| endometriosis, pelvic pain, fibromyalgia | | | | | | |
| CBN | Hemp Distillate | 5.0 | 10 | 1.6 | softgel | Anti-nociceptive, anti-inflammatory, |
| | | | | | | anxiolytic, estrogen attenuating |
| CBD | Hemp Distillate | 15.0 | 30 | 4.7 | | Anti-inflammatory, anxiolytic |
| Angelica sinensis | Ferulic Acid, Liquid | 100.0 | 200 | 31.4 | | Estrogen attenuation, antifibrotic, |
| | Extract | | | | | antiproliferative |
| Ligusticum chuanxiong | Ligustrazine, Liquid | 80.0 | 160 | 25.2 | | Anti-inflammatory, anti-neuropathic, |
| | Extract | | | | | neurological support |
| Corydalis yanhusuo | 50% THP, Liquid | 90.0 | 180 | 28.3 | | Antinociceptive, opioidergic |
| | Extract | | | | | |
| Luteolin | Isolate | 25.0 | 50 | 7.9 | | Improves brain fog, neuropathic pain |
| Piper nigrum | CO2 Extract | 3.0 | 6 | 0.9 | | Anesthetic, CB2 agonist, |
| | | | | | | antinociceptive, AEA/2-AG reuptake |
| | | | | | | inhibitor |
| Total | | 318.0 | 636 | 100.0 | | |
| SOFTGEL formulation P60 | | | | | | |
| Hypothyroid Male | | | | | | |
| CBN | Hemp Distillate | 5.0 | 10 | 1.6 | softgel | Anti-nociceptive, anti-inflammatory, |
| | | | | | | anxiolytic |
| CBD | Hemp Distillate | 15.0 | 30 | 4.9 | | Anti-inflammatory, anxiolytic |
| Salix alba | 25% Salicin | 125.0 | 250 | 41.2 | | COX inhibitor, antiflammatory |
| | derivatives | | | | | |
| Zinc | Zinc Citrate | 5.0 | 10 | 1.6 | | Nutrient |
| Cholecalciferol | 400 IU | 0.01 | 0.02 | 0.0033 | | Immune support, improved thyroid |
| | | | | | | function in autoimmune |
| Commiphora mukul | Standardized to | 150.0 | 300 | 49.5 | | Thyroid stimulation, antiinflammatory |
| | guggulsterones | | | | | |
| | (KSM) | | | | | |
| Selenium | L-selenomethione | 0.1 | 0.2 | 0.033 | | Nutrient |
| Iodine | Potassium Iodide | 0.075 | 0.15 | 0.025 | | Nutrient |
| Piper nigrum | CO2 Extract | 3.0 | 6.0 | 1.0 | | Anesthetic, CB2 agonist, |
| | | | | | | antinociceptive, AEA/2-AG reuptake |
| | | | | | | inhibitor |
| Total | | 303.2 | 606.4 | 100.00 | | |
| SOFTGEL formulation P62 | | | | | | |
| Hypothyroid Female | | | | | | |
| CBN | Hemp Distillate | 2.5 | 5 | 0.8 | softgel | Anti-nociceptive, anti-inflammatory, |
| | | | | | | anxiolytic, estrogen attenuating |
| CBD | Hemp Distillate | 15.0 | 30 | 4.6 | | Anti-inflammatory, anxiolytic |
| Salix alba | 25% salicin | 125.0 | 250 | 41.229 | | COX inhibitor, antiflammatory |
| | derivatives | | | | | |
| Zinc | Zinc citrate | 5.0 | 10 | | | Nutrient |
| Cholecalciferol | 400 IU | 0.01 | 0.02 | 0.003 | | Nutrient, immunomodulation, |
| | | | | | | antinociceptive, anti-inflammatory |
| Commiphora mukul | Standardized to | 150.0 | 300 | 46.1 | | Thyroid stimulation, anti-inflammatory |
| | guggulsterones | | | | | |
| Pueraria lobata | 50% puerarin | 25.0 | 50 | 7.7 | from plant | Analgesic, estrogenic |
| | | | | | source | |
| Selenium | L-Selenomethione | 0.1 | 0.2 | 0.033 | | Nutrient |
| Iodine | Potassium Iodide | 0.075 | 0.15 | 0.025 | | Nutrient |
| Piper nigrum | CO2 Extract | 3.0 | 6 | 0.9 | | Anesthetic, CB2 agonist, |
| | | | | | | antinociceptive, AEA/2-AG reuptake |
| | | | | | | inhibitor |
| Total | | 325.7 | 651.4 | 100.0 | | |
| SOFTGEL formulation P64 | | | | | | |
| menses, antispasmodic musculoskeletal | | | | | | |
| spasm | | | | | | |
| CBN | Hemp Distillate | 5.0 | 10 | 1.6 | softgel | Anti-nociceptive, anti-spasmodic, anti- |
| | | | | | | inflammatory, anxiolytic |
| CBD | Hemp Distillate | 15.0 | 30 | 4.8 | | Antispasmodic, anxiolytic |
| Viburnum prunifolium | Liquid Extract | 150.0 | 300 | 47.9 | | Antispasmodic, analgesic, uterine |
| | | | | | | relaxant, antinociceptive |
| Matricaria recutita | Liquid Extract | 75.00 | 150 | 24.0 | | Antispasmodic, analgesic, anxiolytic |
| Paeonia lactiflora | standardized to | 30 | 60 | 1.6 | | Antispasmodic, antinociceptive |
| | paeoniflorin | | | | | |
| Melissa officinalis | Essential oil | 25.0 | 50 | 8.0 | | Analgesic, antispasmodic, |
| | | | | | | antiinflammatory, anxiolytic |
| Dioscorea villosa | Liquid Extract | 25.0 | 50 | 8.0 | | Antispasmodic, cytokine inhibition, |
| | | | | | | anti-inflammatory, analgesic |
| Zingiber officinalis | Liquid Extract | 10.0 | 20 | 3.2 | | Warming, antispasmodic, antinausea, |
| | | | | | | antiinflammatory |
| Piper nigrum | CO2 Extract | 3.0 | 6 | 1.0 | | Anesthetic, CB2 agonist, |
| | | | | | | antinociceptive, AEA/2-AG reuptake |
| | | | | | | inhibitor |
| Total | | 338.0 | 676.0 | 100.0 | | |

*cap = capsule

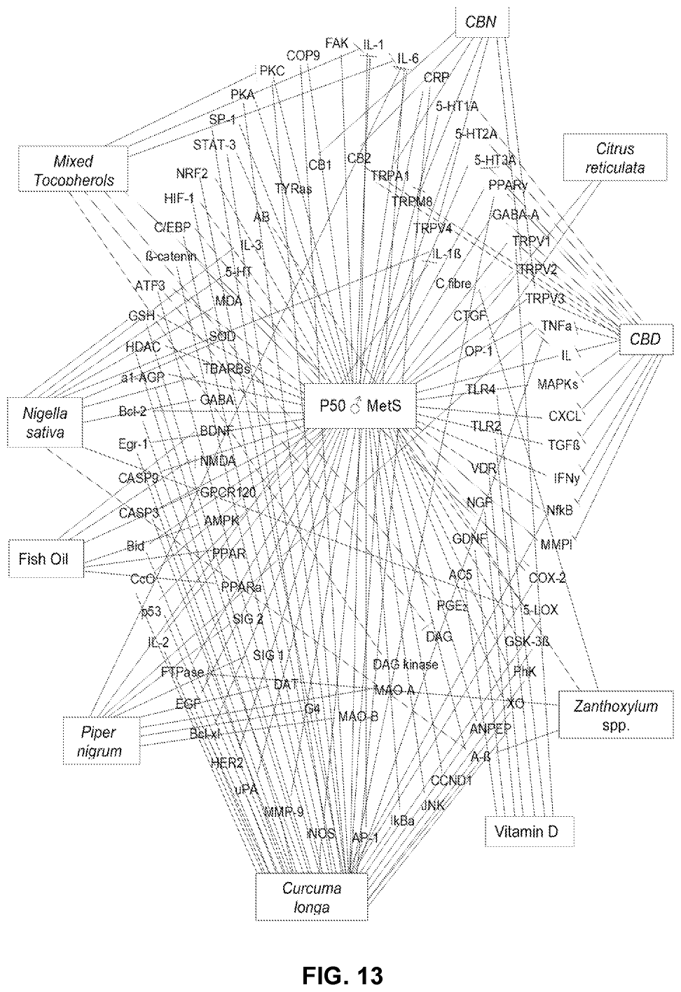

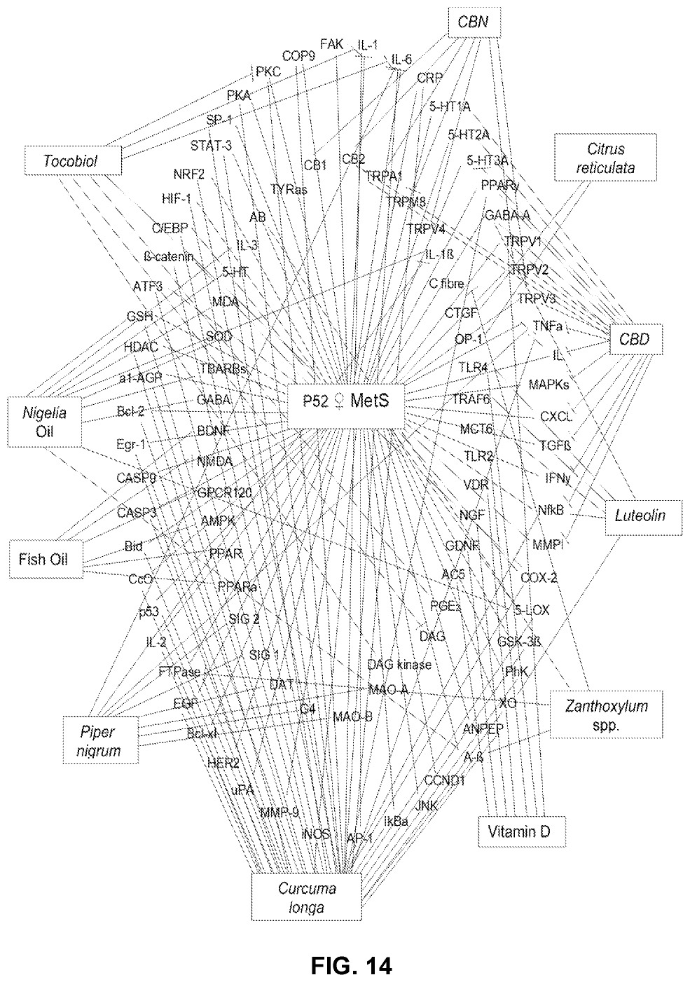

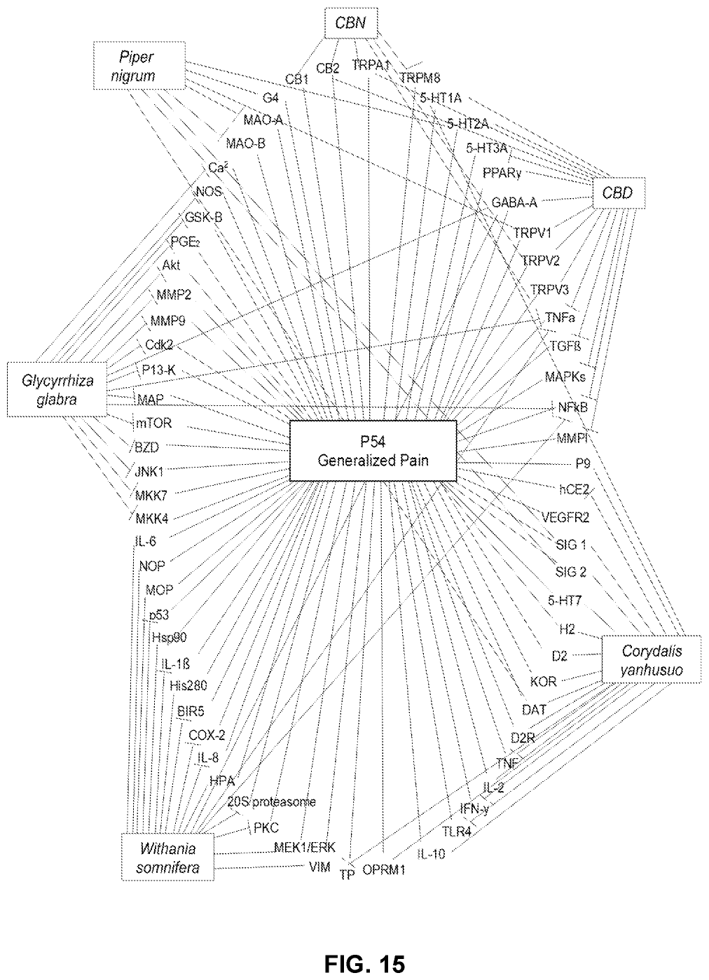

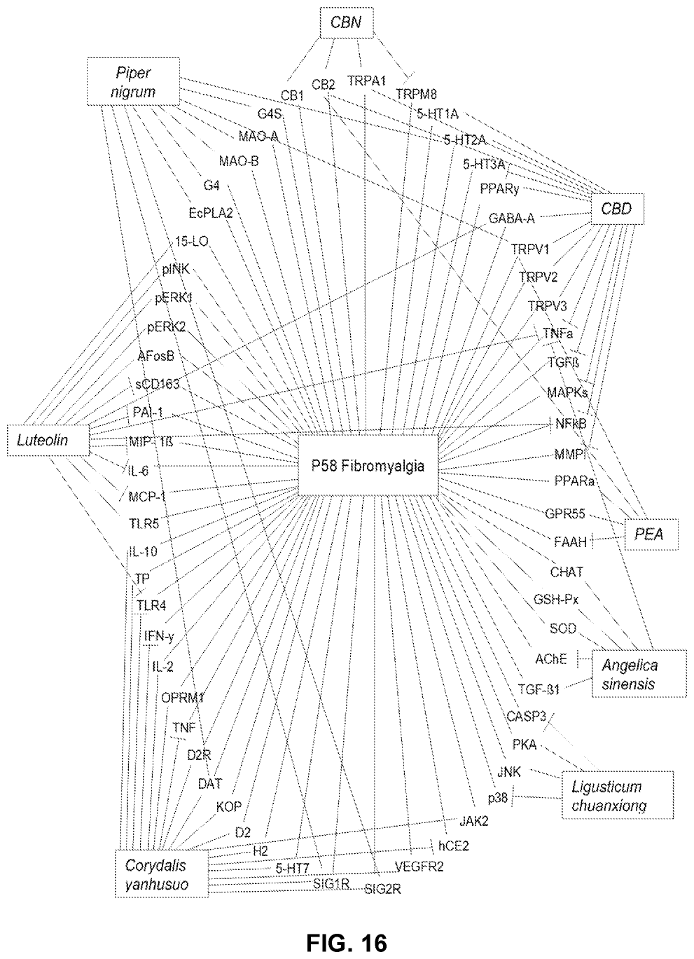

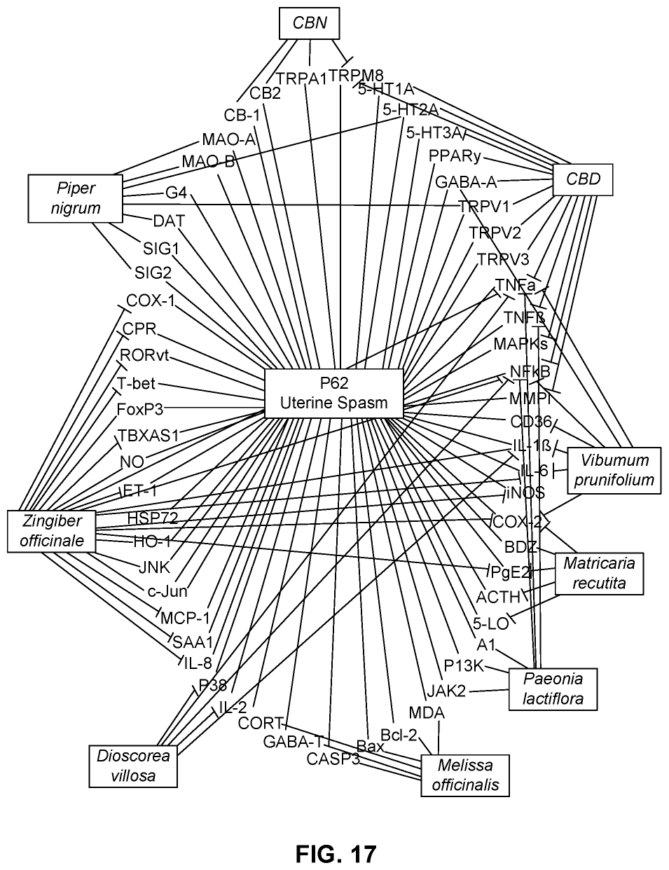

FIGS. 13-17 show examples of how the compositions of the present disclosure (e.g., pain formulation P50 (FIG. 13), pain formulation P52 (FIG. 14), pain formulation P54 (FIG. 15), pain formulation P58 (FIG. 16), and pain formulation P62 (FIG. 17)), target multiple targets on the ECS and other enzymes and receptors that help control sleep, pain, and/or anxiety.

Exemplary anxiety formulations are described in Table 4.

TABLE 4

| | Ingredients | 1 cap | 2 caps | % | | RATIO |
|---|---|---|---|---|---|---|
| SOFTGEL | CBN | 1 | 2 | 0.48% | distillate or isolate | 20 |
| formulation | CBD | 20 | 40 | 9.57% | distillate or isolate | |
| C30 | Lavandula officinalis EO | 30 | 60 | 14.35% | Lavela WS 1265 | |
| Social | Piper methysticum | 30 | 60 | 14.35% | viscous material | |
| Anxiety | Withania somnifera | 125 | 250 | 59.81% | Liquid ext | |
| Disorder | Piper nigrum extract | 3 | 6 | 1.44% | CO2 extract | |
| | | 209 | 418 | 100.00% | | |
| | Ingredients | 1 cap | 2 caps | % | | RATIO |
| SOFTGEL | CBN | 2.5 | 5 | 0.79% | distillate or isolate | 10 |
| formulation | CBD | 25 | 50 | 7.92% | distillate or isolate | |
| C32 | Rutaecarpine | 50 | 100 | 15.85% | 98% | |
| Anxiety due | Passiflora incarnata | 40 | 80 | 12.68% | Liquid ext | |
| to caffeine | Ganoderma lucidum | 175 | 350 | 55.47% | Liquid ext | |
| excess | Magnolia officinalis | 20 | 40 | 6.34% | Liquid ext, 50% | |
| | | | | | honokiol/magnolol | |
| | Piper nigrum extract | 3 | 6 | 0.95% | CO2 extract | |
| | TOTAL | 315.5 | 631 | 100.00% | | |
| | Ingredients | 1 cap | 2 caps | % | | RATIO |
| SOFTGEL | CBN | 2 | 4 | 0.75% | distillate or isolate | 10 |
| formulation | CBD | 20 | 40 | 7.55% | distillate or isolate | 4 |
| C34 | CBG | 5 | 10 | 1.89% | distillate or isolate | |
| Generalized | Matricaria recutita | 100 | 200 | 37.74% | Liquid ext | |
| Anxiety | Pueraria lobata | 30 | 60 | 11.32% | pwd | |
| Disorder | Zizyphus spinosa | 75 | 150 | 28.30% | pwd | |
| Female | Sceletium tortuosum | 8 | 16 | 3.02% | lipophilic ext | |
| | Citrus reticulata (e.g., | 25 | 50 | 9.43% | Essential oil | |
| | Citrus reticulata Blanco) | | | | | |
| | TOTAL | 265 | 530 | 100.00% | | |
| | | 1 cap | 2 caps | % | | RATIO |
| SOFTGEL | CBG | 4 | 8 | 1.38% | distillate or isolate | 5 |
| formulation | CBD | 20 | 40 | 6.92% | distillate or isolate | |
| C36 | Withania somnifera | 200 | 400 | 69.20% | Liquid ext | |
| Anxiety | Crocus sativus | 15 | 30 | 5.19% | pwd | |
| induced | Polygala tenuifolia | 25 | 50 | 8.65% | pwd | |
| depression | Citrus reticulata (e.g., | 25 | 50 | 8.65% | Essential oil | |
| | Citrus reticulata Blanco) | | | | | |
| | TOTAL | 289 | 578 | 100.00% | | |
| | | 1 cap | 2 caps | % | | RATIO |
| SOFTGEL | CBD | 25 | 50 | 8.12% | distillate or isolate | 5 |
| formulation | CBG | 5 | 10 | 1.62% | distillate or isolate | |
| C38 | Schisandra chinensis | 150 | 300 | 48.70% | Liquid ext | |
| Anxiety in | Dihydromyricetin | 125 | 250 | 40.58% | isolate | |
| Alcoholism | Piper nigrum | 3 | 6 | 0.97% | CO2 extract | |
| | Totals | 308 | 616 | 100.00% | | |
| | | 1 cap | 2 caps | % | | RATIO |
| SOFTGEL | CBN | 5 | 10 | 1.62% | distillate or isolate | 5 |
| formulation | CBD | 25 | 50 | 8.12% | distillate or isolate | |
| C42 | 5-HTP | 25 | 50 | 8.12% | isolate | |
| PTSD | Ziziphus jujuba semen | 125 | 250 | 40.58% | pwd | |
| | (Z. spinosa) | | | | | |
| | Poria cocos sclerotium | 125 | 250 | 40.58% | Liquid ext | |
| | Piper nigrum | 3 | 6 | 0.97% | CO2 extract | |
| | Totals | 308 | 616 | 100.00% | | |
| | | 1 cap | 2 caps | % | | RATIO |
| SOFTGEL | CBN | 2.5 | 5 | 0.83% | distillate or isolate | 10 |
| formulation | CBD | 25 | 50 | 8.32% | distillate or isolate | 5 |
| C44 | CBG | 5 | 10 | 1.66% | distillate or isolate | |
| Anxiety in | Panax ginseng radix | 50 | 100 | 16.64% | Liquid ext | |
| older females | Pueraria lobata | 50 | 100 | 16.64% | pwd | |
| | Centella asiatica | 165 | 330 | 54.91% | Liquid ext | |
| | Piper nigrum | 3 | 6 | 1.00% | CO2 extract | |
| | TOTAL | 300.5 | 601 | 100.00% | | |
| | | 1 cap | 2 caps | % | | RATIO |
| SOFTGEL | CBN | 2.5 | 5 | 0.81% | distillate or isolate | 10 |
| formulation | CBD | 25 | 50 | 8.05% | distillate or isolate | 5 |
| C40 | CBG | 5 | 10 | 1.61% | distillate or isolate | |
| Anxiety in | Panax ginseng radix | 75 | 150 | 24.15% | Liquid ext | |
| older males | Ganoderma lucidum | 150 | 300 | 48.31% | Liquid ext | |
| | Paeonol | 50 | 100 | 16.10% | isolate | |
| | Piper nigrum | 3 | 6 | 0.97% | CO2 extract | |
| | TOTAL | 310.5 | 621 | 100.00% | | |
| | Specific Phobia | 1 cap | 2 caps | % | | RATIO |
| SOFTGEL | CBN | 2.5 | 5 | 0.71% | distillate or isolate | 8 |
| formulation | CBD | 20 | 40 | 5.71% | distillate or isolate | |
| C46 | Passiflora incarnata | 100 | 200 | 28.53% | Liquid ext | |
| Specific | Scutellaria lateriflora | 100 | 200 | 28.53% | Liquid ext | |
| Phobia | Lycopus spp. | 100 | 200 | 28.53% | Liquid ext | |
| | Sceletium tortuosum | 25 | 50 | 7.13% | standardized ext | |
| | Piper nigrum | 3 | 6 | 0.86% | CO2 extract | |
| | TOTAL | 350.5 | 701 | 100.00% | | |
| | | 1 cap | 2 caps | % | | RATIO |
| SOFTGEL | CBN | 2.5 | 5 | 1.07% | distillate or isolate | 8 |
| formulation | CBD | 20 | 40 | 8.58% | distillate or isolate | |
| C48 | CBG | 7.5 | 15 | 3.22% | | |
| Obsessive- | Rosmarinus officinalis | 100 | 200 | 42.92% | 45:1 concentrated | |
| Compulsive | | | | | in rosmarinic acid | |
| Disorder | Valeriana officinalis | 50 | 100 | 21.46% | valerenic acid 0.8% | |
| | Theacrine | 50 | 100 | 21.46% | isolate | |
| | Piper nigrum | 3 | 6 | 1.29% | CO2 extract | |
| | Totals | 233 | 466 | 100.00% | | |
| | | 1 cap | 2 caps | % | | RATIO |
| SOFTGEL | CBG | 7.5 | 15 | 2.91% | distillate or isolate | 1.3 |
| formulation | CBD | 10 | 20 | 3.88% | distillate or isolate | |
| C49 | Matricaria | 75 | 150 | 29.13% | standardized to | |
| Generalized | | | | | apigenin, bisabolol | |
| Anxiety | Zizyphus spinosa | 75 | 150 | 29.13% | 2% triterpenes | |
| Disorder in | Piper methysticum | 40 | 80 | 15.53% | extract 50% | |
| Male | | | | | kavalactones | |
| | Citrus reticulata (e.g., | 50 | 100 | 19.42% | Essential oil | |
| | Citrus reticulata Blanco) | | | | | |
| | TOTAL | 257.5 | 515 | 100.00% | | |

*cap = capsule

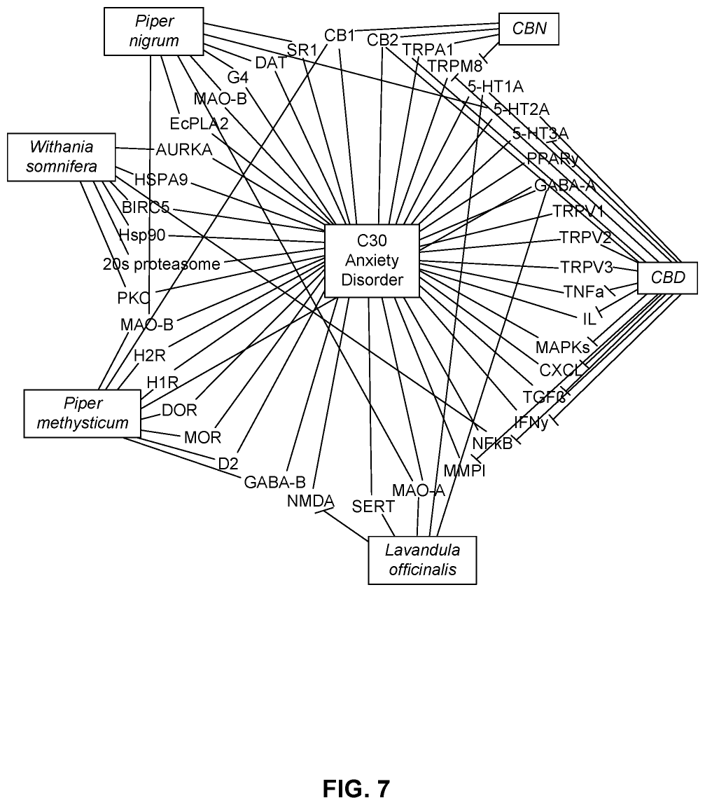

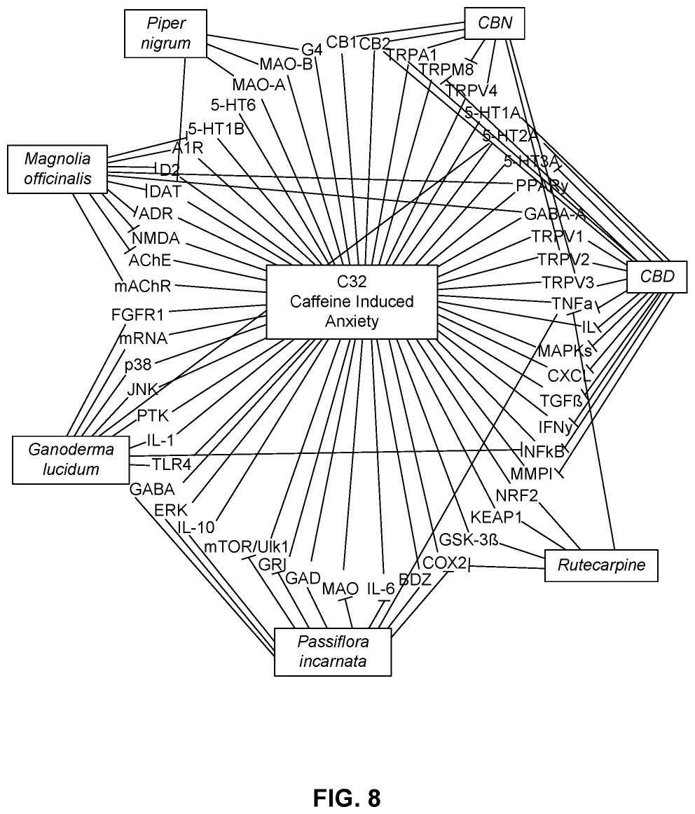

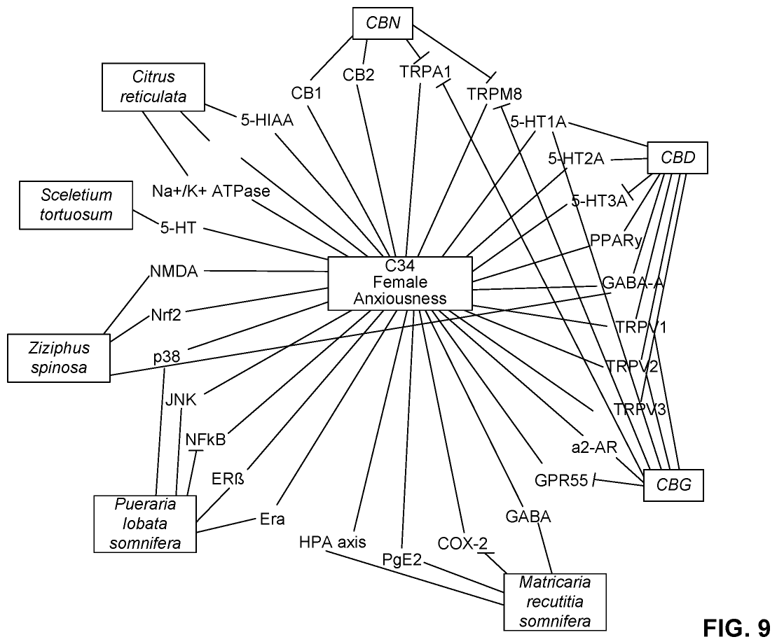

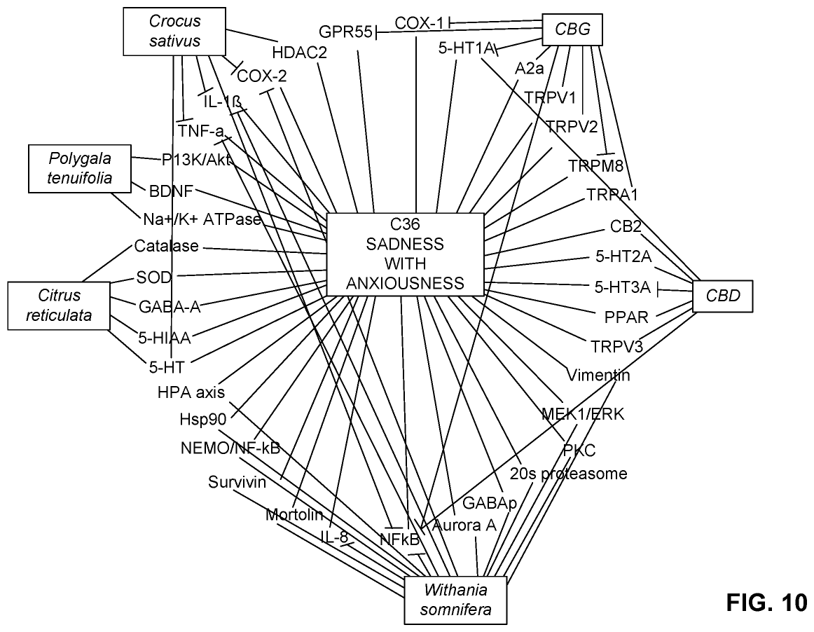

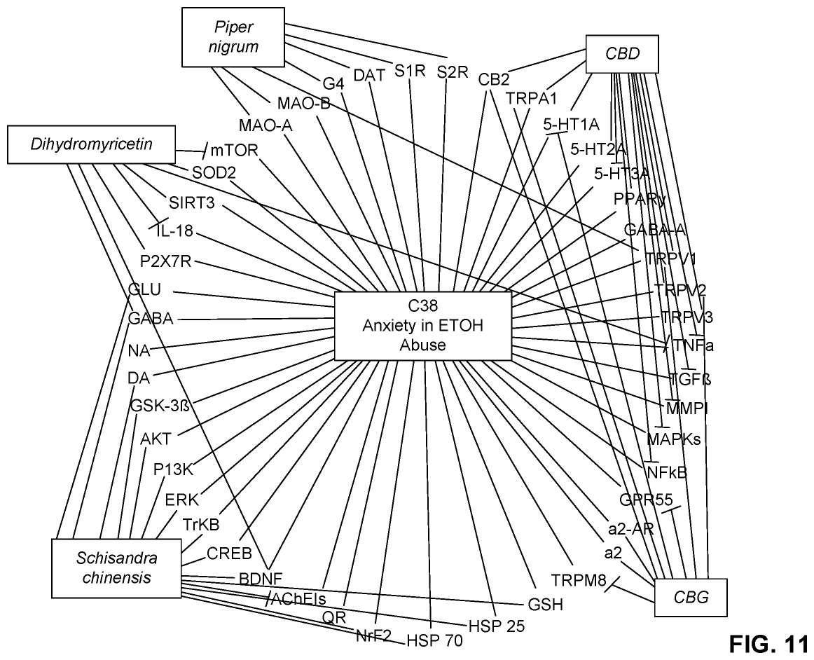

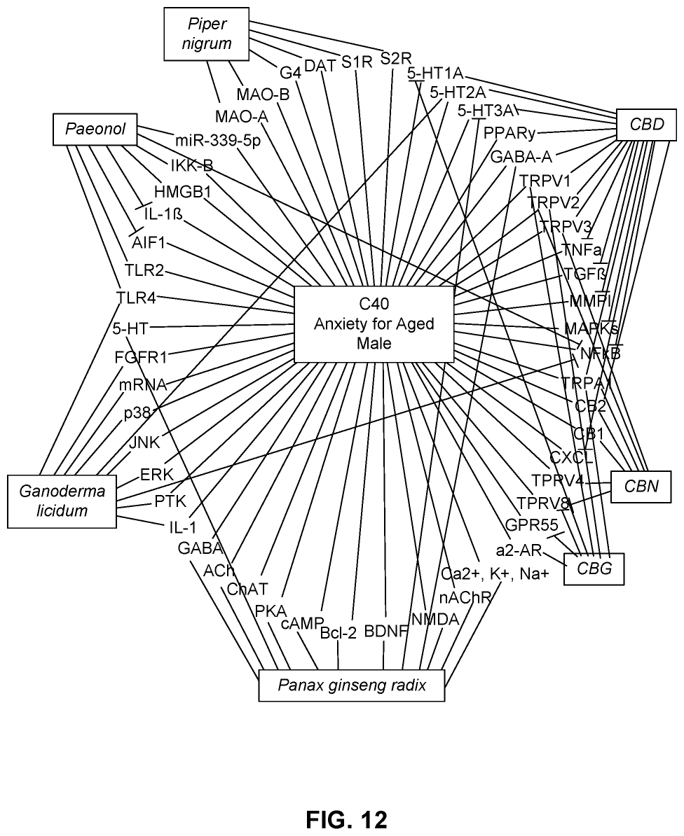

FIGS. 7-12 show examples of how the compositions of the present disclosure (e.g., anxiety formulation C30 (FIG. 7), anxiety formulation C32 (FIG. 8), anxiety formulation C34 (FIG. 9), anxiety formulation C36 (FIG. 10), anxiety formulation C38 (FIG. 11), anxiety formulation C40 (FIG. 12)), target multiple targets on the ECS and other enzymes and receptors that help control sleep, pain, and/or anxiety.

In some aspects, the compositions of the present disclosure (e.g., as shown in Tables 1, 3, and 4) further comprise about 5 mg to about 175 mg of medium-chain triglyceride (MCT) oil, about 5 mg to about 100 mg of beewax (e.g., yellow USP), about 1 mg to about 30 mg sunflower lecithin, about 0 mg to about 30 mg glycerin (e.g., 99.6%, USP, vegetable-based), or any combination thereof.

### Example 2—Personalization of Cannabinoid Therapy Formulations

After completing a short online assessment, consumers received a free personalized wellness guide that included insights and education on the specific ingredients and compositions matched to its individual needs. The compositions of the present disclosure were customized taking into consideration health condition, severity of condition, age, gender, and lifestyle of an individual. For each consumer, two formulations were selected that best match their needs. The consumer tried a 5 day sample of each formulation (e.g., five days of sleep formulation #2 and five days of sleep formulation #6), kept track of their daily results, and then had an option to order a full size of the formulation that worked best for the consumer.

Out of 104 consumers that participated in the efficacy study, 13 received sleep formulations only (Panel 1), 22 received anxiety formulations only (Panel 2), and 69 received both sleep and anxiety formulations (Panel 3).

Panel 1 efficacy study included 12 out of 13 consumers. One panelist was excluded from the sleep quality and improvement analysis because he/she indicated having an extremely good sleep quality pre-trial. The consumers completed the single choice type questions.

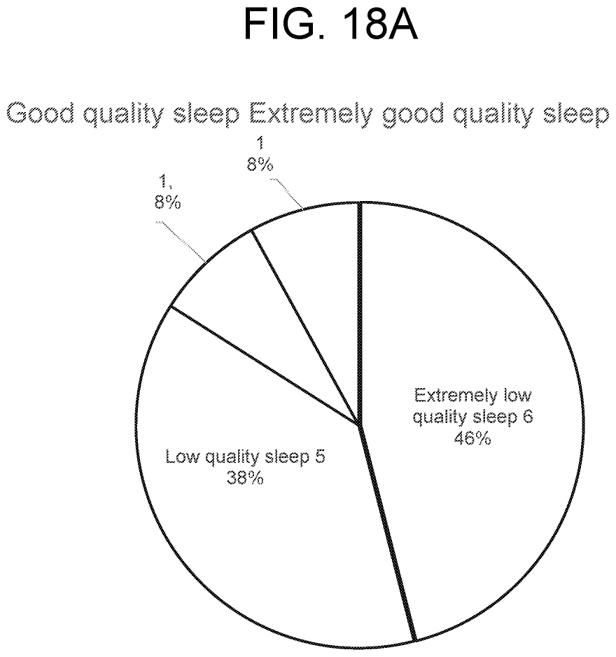

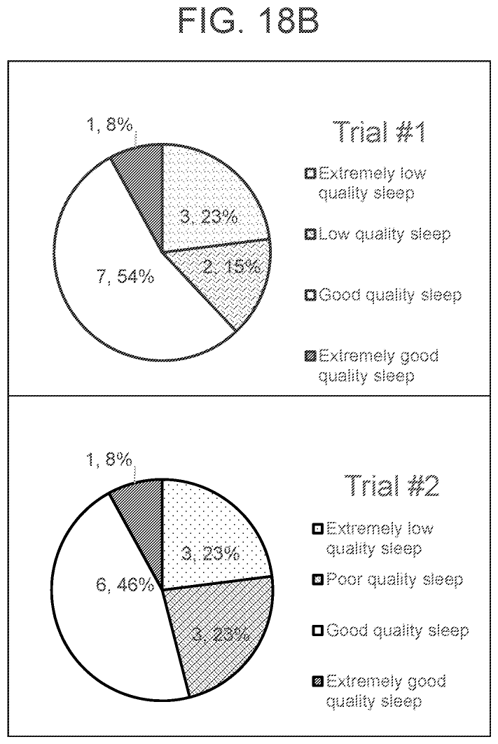

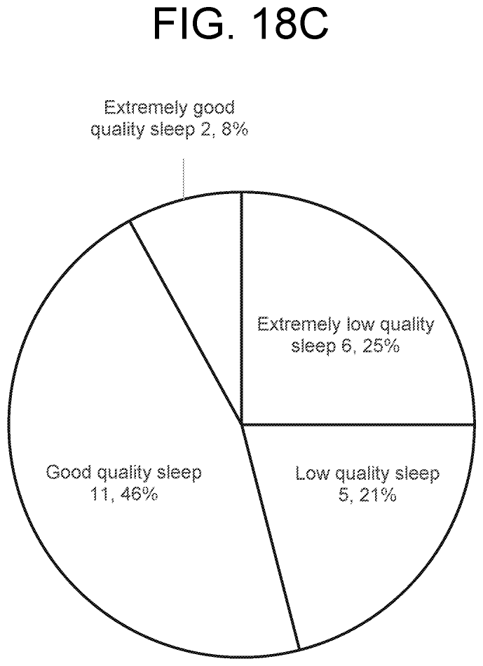

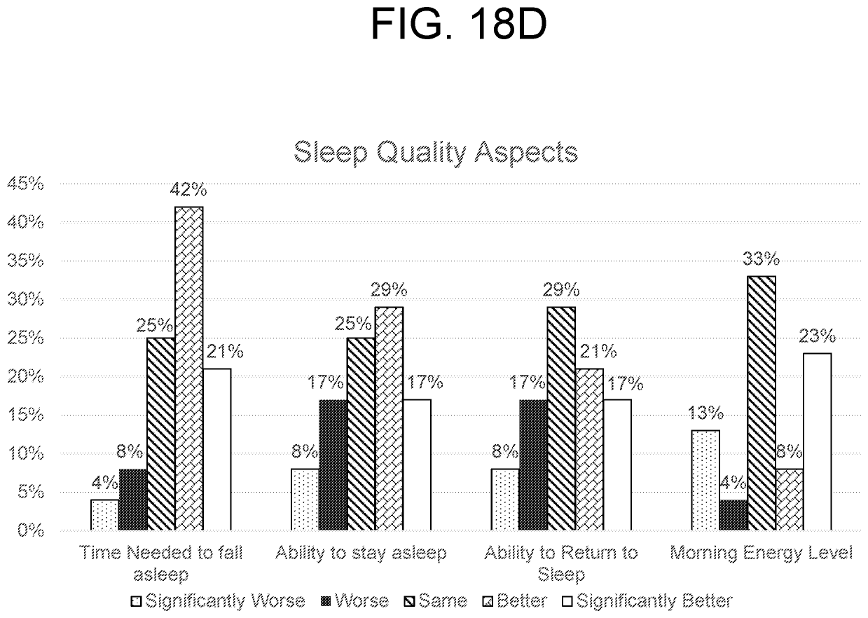

FIG. 18A shows that pre-trial, 84% of panelists that received sleep formulations indicated that they experienced low or extremely low-quality sleep and 8% of panelists indicated they experienced good sleep quality pre-trial. After trying both Trial #1 and Trial #2 sleep formulations, 54% of participants indicated they experienced good or extremely good quality sleep, while only 46% indicated their sleep was low quality or extremely low quality. (see FIG. 18C). As shown in Table 5 below, sleep quality improvement over pre-trial baseline for this segment was 61.5% (improvement measured from extremely low quality to low quality, low quality to high quality, and high quality to extremely high quality).

TABLE 5

| | Sleep quality aspects | | | | | | | | |
|---|---|---|---|---|---|---|---|---|---|
| | Non-Cumulative | | | Cumulative | | | Cumlative % | | |
| Best | 1st | 2nd | Best | 1st | 2nd | Best | 1st | 2nd | Best |
| 3-level Improvement | 0 | 0 | 0 | 0 | 0 | 0 | 0.0% | 0.0% | 0.0% |
| 2-level Improvement | 3 | 2 | 3 | 3 | 2 | 3 | 23.1% | 15.4% | 23.1% |
| 1-level Improvement | 5 | 4 | 5 | 8 | 6 | 8 | 61.5% | 46.2% | 61.5% |
| No Improvement | 5 | 7 | 5 | 13 | 13 | 13 | | | |
| TOTAL | 13 | 13 | 13 | | | | | | |

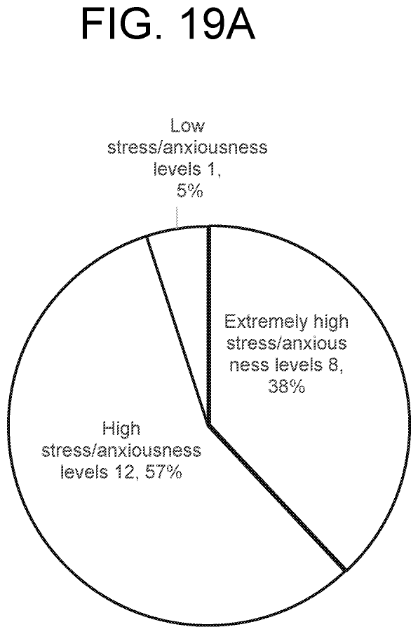

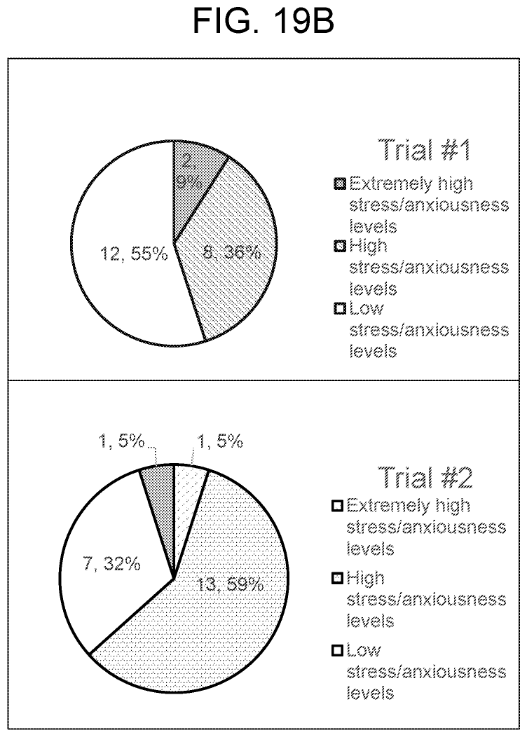

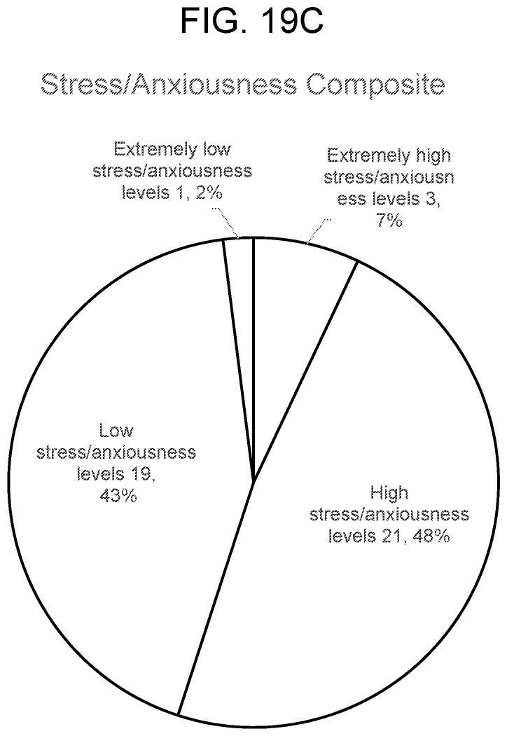

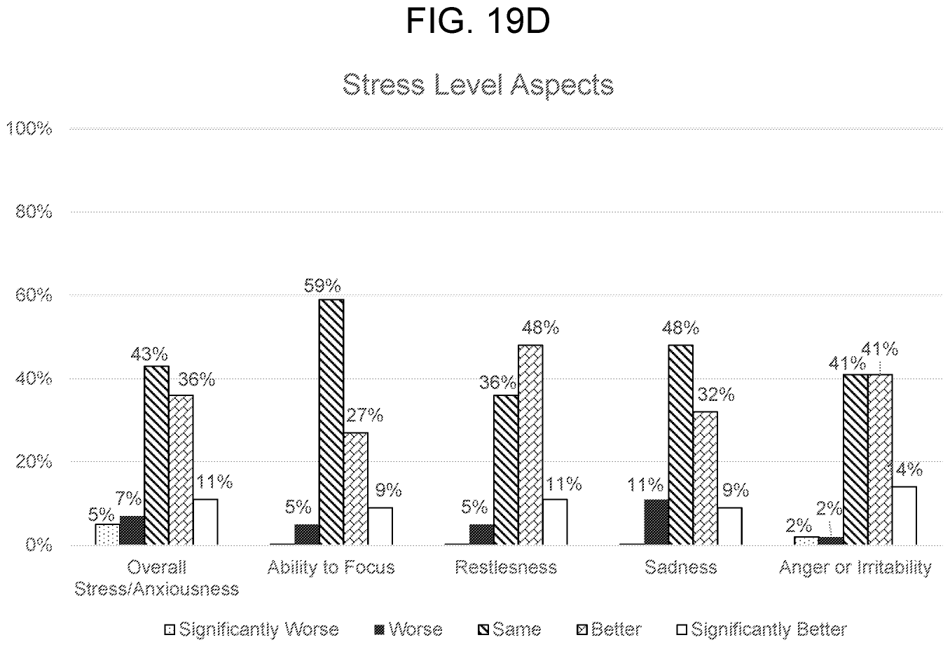

Panelists reported the following aspects of their sleep experience: 31% reported increased morning energy level; 38% reported increased ability to return to sleep if awakened; 46% reported increased ability to stay asleep; and 63% reported time needed to fall asleep decreased (see FIG. 18D). Panel 2 efficacy study included 22 consumers. The consumers completed the single choice type questions. FIG. 19A shows that pre-trial, 95% of panelists that received anxiety formulations indicated that they experienced high or extremely high levels of stress/anxiousness and 5% of panelists indicated they experienced low stress pre-trial. No panelists indicated they had extremely low stress/anxiousness pre-trial (0). After trying both Trial #1 and Trial #2 anxiety formulations, 45% of participants indicated they experienced low or extremely low stress/anxiousness levels, while 55% indicated their stress level was high or extremely high. (see FIG. 19C). As shown in Table 6 below, stress level improvement over the pre-trial baseline for this segment was 72.7% (measured from extremely high levels to high levels, high levels to low levels, and low levels to extremely low levels).

TABLE 6

| | Stress level aspects | | | | | | | | |
|---|---|---|---|---|---|---|---|---|---|
| | Non-Cumulative | | | Cumulative | | | Cumlative % | | |
| Best | 1st | 2nd | Best | 1st | 2nd | Best | 1st | 2nd | Best |
| 3-level Improvement | 0 | 1 | 1 | 0 | 1 | 1 | 0.0% | 4.5% | 4.5% |
| 2-level Improvement | 4 | 1 | 3 | 4 | 2 | 4 | 18.2% | 9.1% | 18.2% |
| 1-level Improvement | 9 | 10 | 12 | 13 | 12 | 16 | 59.1% | 54.5% | 72.7% |
| No Improvement | 9 | 10 | 6 | 22 | 22 | 22 | | | |
| TOTAL | 22 | 22 | 22 | | | | | | |

Panelists reported the following aspects of their stress levels: 47% reported decreased levels of overall stress/anxiousness; 36% reported increased ability focus; 59% reported decrease in restlessness; 41% reported decrease in sadness; and 55% reported decrease in anger or irritability (see FIG. 19D).

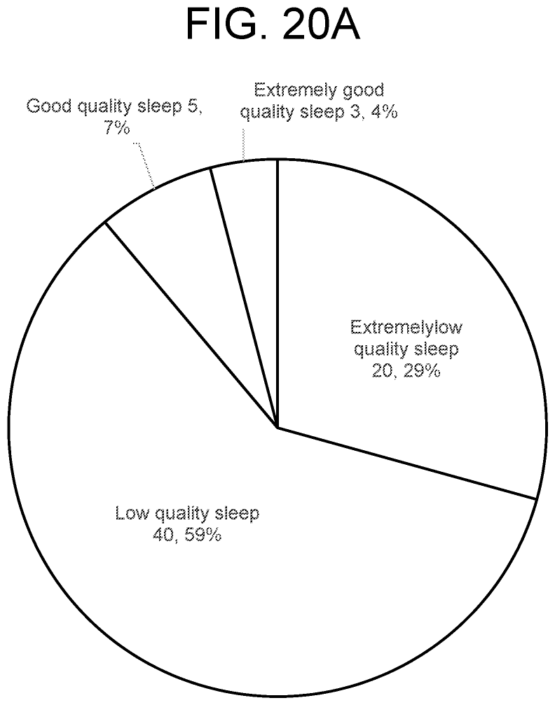

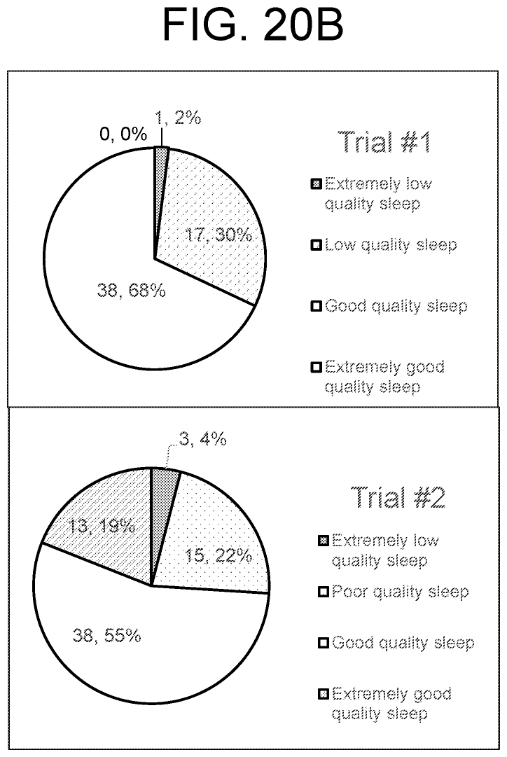

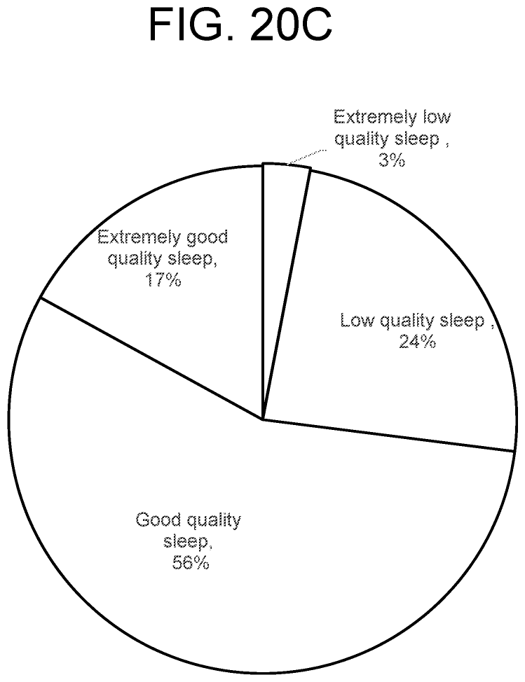

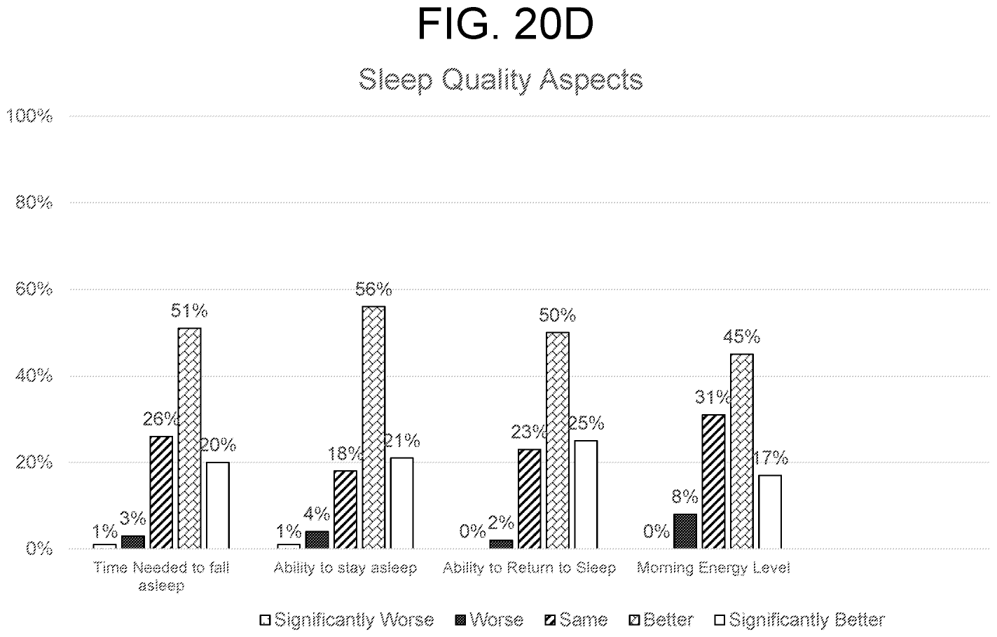

Panel 3, part I efficacy study included 66 out of 69 consumers. Panelists that indicated they had extremely good quality sleep pre-trial were excluded from sleep quality and improvement metrics (3 total). The consumers completed the single choice type questions. FIG. 20A shows that pre-trial, 88% of panelists that received both sleep and anxiety formulations indicated that they experienced low or extremely low-quality sleep and 7% of panelists indicated they experienced good sleep quality pre-trial. After trying both sleep trial formulations in conjunction with the anxiety formulations, 73% of participants indicated that they experienced good or extremely good quality sleep, while only 27% indicated their sleep was low quality or extremely low quality (see FIG. 20C). As shown in Table 7, sleep quality improvement overall for this segment was 86.4% (measured from extremely low quality to low quality, low quality to high quality, and high quality to extremely high quality).

TABLE 7

| | Sleep quality aspects | | | | | | | | |
|---|---|---|---|---|---|---|---|---|---|
| | Non-Cumulative | | | Cumulative | | | Cumlative % | | |
| Best | 1st | 2nd | Best | 1st | 2nd | Best | 1st | 2nd | Best |
| 3-level Improvement | 3 | 6 | 6 | 3 | 6 | 6 | 4.5% | 9.2% | 9.1% |
| 2-level Improvement | 14 | 11 | 17 | 17 | 17 | 23 | 25.8% | 26.2% | 34.8% |
| 1-level Improvement | 34 | 32 | 34 | 51 | 49 | 57 | 77.3% | 75.4% | 86.4% |
| No Improvement | 15 | 16 | 9 | 66 | 65 | 66 | | | |
| TOTAL | 66 | 65 | 66 | | | | | | |

Panelists reported the following aspects of their sleep experience: 62% reported increased morning energy level; 75% reported increased ability to return to sleep if awakened; 77% reported increased ability to stay asleep; and 71% reported time needed to fall asleep decreased (see FIG. 20D).

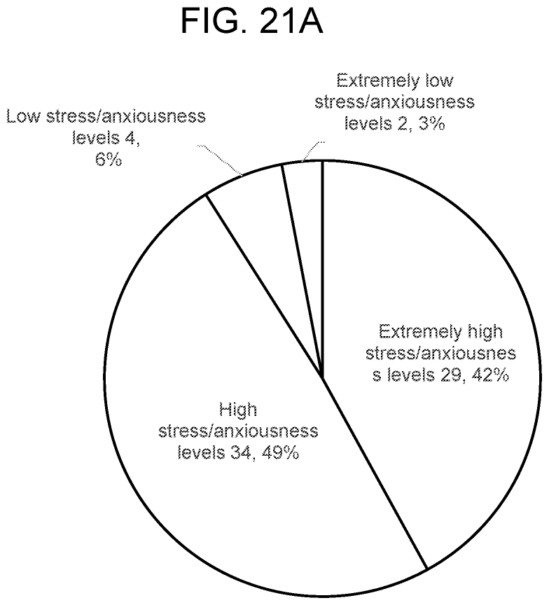

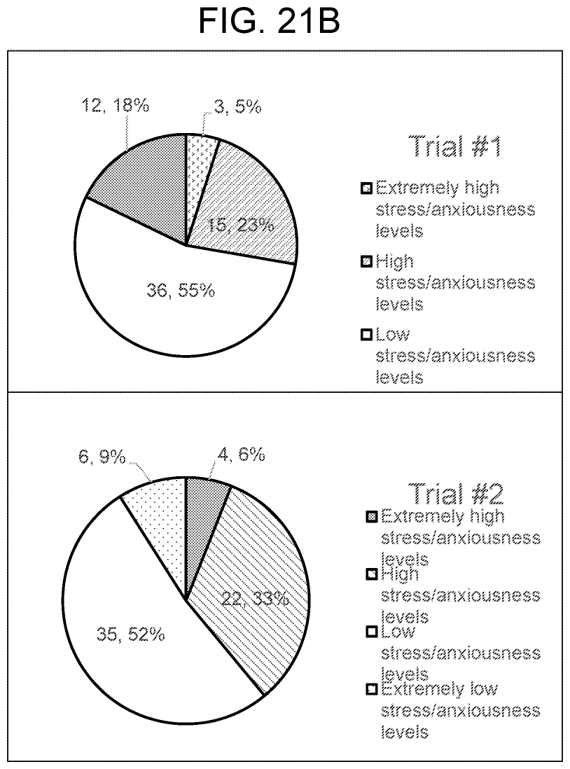

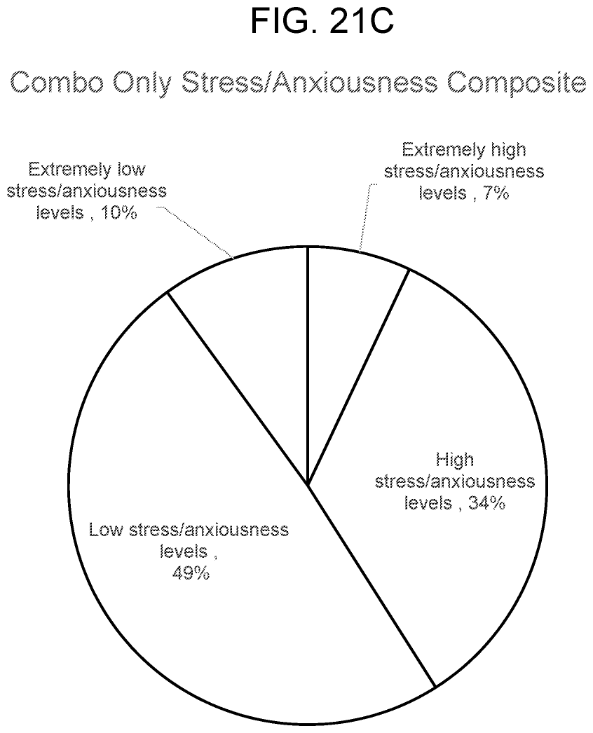

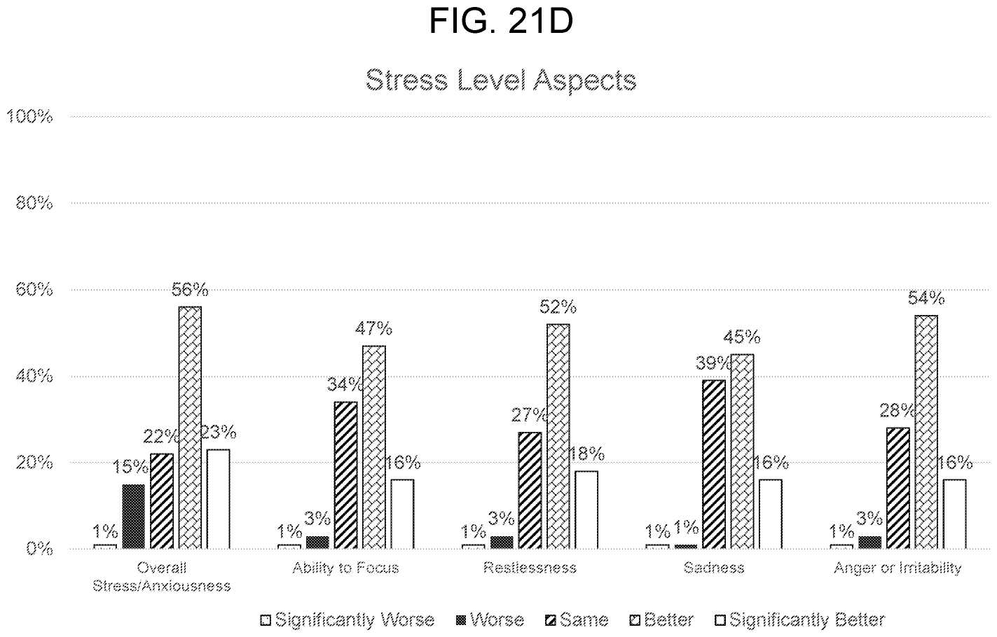

Panel 3, part I efficacy study included 67 out of 69 consumers. Two panelists indicated they had extremely low stress/anxiousness pre-trial and were not included in efficacy or improvement metrics as a decrease in stress/anxiousness levels could not be measured. The consumers completed the single choice type questions. FIG. 21A shows that pre-trial, 91% of panelists that received both sleep and anxiety formulations indicated that they experienced high or extremely high levels of stress/anxiousness and 9% of panelists indicated they experienced low stress pre-trial. After trying both sleep trial formulations in conjunction with the anxiety formulations, 59% of participants indicated they experienced low or extremely low stress/anxiousness levels, while 41% indicated their stress level was high or extremely high. (see FIG. 21C). As shown in Table 8, stress level improvement overall for this segment was 73.1% (measured from extremely high levels to high levels, high levels to low levels, and low levels to extremely low levels).

TABLE 8

| | Stress level aspects | | | | | | | | |
|---|---|---|---|---|---|---|---|---|---|
| | Non-Cumulative | | | Cumulative | | | Cumlative % | | |
| Best | 1st | 2nd | Best | 1st | 2nd | Best | 1st | 2nd | Best |
| 3-level Improvement | 1 | 2 | 3 | 1 | 2 | 3 | 1.5% | 3.0% | 4.5% |
| 2-level Improvement | 21 | 17 | 25 | 22 | 19 | 28 | 32.8% | 28.4% | 41.8% |
| 1-level Improvement | 23 | 24 | 21 | 45 | 43 | 49 | 67.2% | 64.2% | 73.1% |
| No Improvement | 22 | 24 | 18 | 67 | 67 | 67 | | | |
| TOTAL | 67 | 67 | 67 | | | | | | |

Panelists reported the following aspects of their stress levels: 79% reported decreased levels of overall stress/anxiousness; 63% reported increased ability focus; 70% reported decrease in restlessness; 61% reported decrease in sadness; and 70% reported a decrease in anger or irritability (see FIG. 21D).

1.-3. (canceled)

4. A composition comprising:
(i) about 5 mg to about 80 mg of cannabidiol (CBD), about 1 mg to about 15 mg of cannabinol (CBN), about 1 mg to about 30 mg of cannabigerol (CBG), or any combinations thereof;
(ii) one or more of the following: about 40 mg to 260 mg of Ziziphus spinosa, about 0.1 mg to 4 mg of melatonin, and about 30 mg to about 200 mg of Passiflora incarnata; and
(iii) three or more of the following: about 30 mg to about 150 mg of eicosapentaenoic acid (EPA)/docosahexaenoic acid (DHA), about 1 mg to about 80 mg of Citrus reticulata Blanco, about 50 mg to about 200 mg of Withania somnifera, about 30 mg to about 250 mg of Matricaria recutita, about 30 mg to about 220 mg of Lycopus spp., about 50 mg to about 200 mg of Poria cocos, about 5 mg to about 40 mg of Actaea racemosa, about 10 mg to about 150 mg of Pueraria lobata, about 15 mg to about 70 mg of Anemarrhena asphodeloides, about 30 mg to about 120 mg of phosphatidylserine, about 15 mg to about 70 mg of alpha lipoic acid, about 40 mg to about 150 mg of silymarin, about 100 mg to about 280 mg of Schisandra chinensis, about 5 mg to about 50 mg of Cymbopogon flexuosus essential oil, about 10 mg to about 120 mg of Humulus lupulus, about 50 mg to about 200 mg of Grindelia spp., about 30 mg to about 150 mg of Ligusticum wallichii, about 5 mg to about 50 mg of 5-hydroxytryptophan (5-HTP), about 15 mg to about 70 mg of paeonol, about 15 mg to about 150 mg of Panax ginseng, about 20 mg to about 100 mg of Rehmannia glutinosa, about 50 mg to about 200 mg of Polygala tenuifolia, about 20 mg to about 100 mg of Lavendula officinalis, about 50 mg to about 200 mg of Scutellaria lateriflora, about 10 mg to about 150 mg of Pueraria lobata, about 5 mg to about 70 mg of Sceletium tortuosum, about 1 mg to about 15 mg of mixed Tocopherols, about 10 mg to about 200 mg of Magnolia officinalis, about 20 mg to about 100 mg of Piper methysticum, and about 1 mg to about 10 mg of Piper nigrum.

5. The composition of claim 4, comprising:
(i) about 5 mg to about 80 mg of cannabidiol (CBD), about 1 mg to about 15 mg of cannabinol (CBN), about 1 mg to about 30 mg of cannabigerol (CBG), or any combinations thereof, and about 40 mg to 260 mg of Ziziphus spinosa; and
(ii) four or more of the following: about 0.1 mg to 4 mg of melatonin, about 30 mg to about 200 mg of Passiflora incarnata, about 30 mg to about 150 mg of eicosapentaenoic acid (EPA)/docosahexaenoic acid (DHA), about 1 mg to about 80 mg of Citrus reticulata Blanco, about 50 mg to about 200 mg of Withania somnifera, about 30 mg to about 250 mg of Matricaria recutita, about 30 mg to about 220 mg of Lycopus spp., about 50 mg to about 200 mg of Poria cocos, about 5 mg to about 40 mg of Actaea racemosa, about 10 mg to about 150 mg of Pueraria lobata, about 15 mg to about 70 mg of Anemarrhena asphodeloides, about 30 mg to about 120 mg of phosphatidylserine, about 15 mg to about 70 mg of alpha lipoic acid, about 40 mg to about 150 mg of silymarin, about 100 mg to about 280 mg of Schisandra chinensis, about 5 mg to about 50 mg of Cymbopogon flexuosus essential oil, about 10 mg to about 120 mg of Humulus lupulus, about 50 mg to about 200 mg of Grindelia spp., about 30 mg to about 150 mg of Ligusticum wallichii, about 5 mg to about 50 mg of 5-hydroxytryptophan (5-HTP), about 15 mg to about 70 mg of paeonol, about 15 mg to about 150 mg of Panax ginseng, about 20 mg to about 100 mg of Rehmannia glutinosa, about 50 mg to about 200 mg of Polygala tenuifolia, about 20 mg to about 100 mg of Lavendula officinalis, about 50 mg to about 200 mg of Scutellaria lateriflora, about 10 mg to about 150 mg of Pueraria lobata, about 5 mg to about 70 mg of Sceletium tortuosum, about 1 mg to about 15 mg of mixed Tocopherols, about 10 mg to about 200 mg of Magnolia officinalis, about 20 mg to about 100 mg of Piper methysticum, and about 1 mg to about 10 mg of Piper nigrum.

6. The composition of claim 4, comprising:
(i) about 5 mg to about 80 mg of cannabidiol (CBD), about 1 mg to about 15 mg of cannabinol (CBN), about 1 mg to about 30 mg of cannabigerol (CBG), or any combinations thereof, and about 0.1 mg to 4 mg of melatonin; and
(ii) four or more of the following: about 40 mg to 260 mg of Ziziphus spinosa, about 30 mg to about 200 mg of Passiflora incarnata; about 30 mg to about 150 mg of eicosapentaenoic acid (EPA)/docosahexaenoic acid (DHA), about 1 mg to about 80 mg of Citrus reticulata Blanco, about 50 mg to about 200 mg of Withania somnifera, about 30 mg to about 250 mg of Matricaria recutita, about 30 mg to about 220 mg of Lycopus spp., about 50 mg to about 200 mg of Poria cocos, about 5 mg to about 40 mg of Actaea racemosa, about 10 mg to about 150 mg of Pueraria lobata, about 15 mg to about 70 mg of Anemarrhena asphodeloides, about 30 mg to about 120 mg of phosphatidylserine, about 15 mg to about 70 mg of alpha lipoic acid, about 40 mg to about 150 mg of silymarin, about 100 mg to about 280 mg of Schisandra chinensis, about 5 mg to about 50 mg of Cymbopogon flexuosus essential oil, about 10 mg to about 120 mg of Humulus lupulus, about 50 mg to about 200 mg of Grindelia spp., about 30 mg to about 150 mg of Ligusticum wallichii, about 5 mg to about 50 mg of 5-hydroxytryptophan (5-HTP), about 15 mg to about 70 mg of paeonol, about 15 mg to about 150 mg of Panax ginseng, about 20 mg to about 100 mg of Rehmannia glutinosa, about 50 mg to about 200 mg of Polygala tenuifolia, about 20 mg to about 100 mg of Lavendula officinalis, about 50 mg to about 200 mg of Scutellaria lateriflora, about 10 mg to about 150 mg of Pueraria lobata, about 5 mg to about 70 mg of Sceletium tortuosum, about 1 mg to about 15 mg of mixed Tocopherols, about 10 mg to about 200 mg of Magnolia officinalis, about 20 mg to about 100 mg of Piper methysticum, and about 1 mg to about 10 mg of Piper nigrum.

7. The composition of claim 4, comprising:
(i) about 5 mg to about 80 mg of cannabidiol (CBD), about 1 mg to about 15 mg of cannabinol (CBN), about 1 mg to about 30 mg of cannabigerol (CBG), or any combinations thereof, and about 30 mg to about 200 mg of Passiflora incarnata; and
(ii) four or more of the following: about 0.1 mg to 4 mg of melatonin; about 40 mg to 260 mg of Ziziphus spinosa, about 30 mg to about 150 mg of eicosapentaenoic acid (EPA)/docosahexaenoic acid (DHA), about 1 mg to about 80 mg of Citrus reticulata Blanco, about 50 mg to about 200 mg of Withania somnifera, about 30 mg to about 250 mg of Matricaria recutita, about 30 mg to about 220 mg of Lycopus spp., about 50 mg to about 200 mg of Poria cocos, about 5 mg to about 40 mg of Actaea racemosa, about 10 mg to about 150 mg of Pueraria lobata, about 15 mg to about 70 mg of Anemarrhena asphodeloides, about 30 mg to about 120 mg of phosphatidylserine, about 15 mg to about 70 mg of alpha lipoic acid, about 40 mg to about 150 mg of silymarin, about 100 mg to about 280 mg of Schisandra chinensis, about 5 mg to about 50 mg of Cymbopogon flexuosus essential oil, about 10 mg to about 120 mg of Humulus lupulus, about 50 mg to about 200 mg of Grindelia spp., about 30 mg to about 150 mg of Ligusticum wallichii, about 5 mg to about 50 mg of 5-hydroxytryptophan (5-HTP), about 15 mg to about 70 mg of paeonol, about 15 mg to about 150 mg of Panax ginseng, about 20 mg to about 100 mg of Rehmannia glutinosa, about 50 mg to about 200 mg of Polygala tenuifolia, about 20 mg to about 100 mg of Lavendula officinalis, about 50 mg to about 200 mg of Scutellaria lateriflora, about 50 mg to about 200 mg of Polygala tenuifolia, about 10 mg to about 150 mg of Pueraria lobata, about 5 mg to about 70 mg of Sceletium tortuosum, about 1 mg to about 15 mg of mixed Tocopherols, about 10 mg to about 200 mg of Magnolia officinalis, about 20 mg to about 100 mg of Piper methysticum, and about 1 mg to about 10 mg of Piper nigrum.

8.-15. (canceled)

16. A method for improving sleep quality in a subject, comprising administering to the subject the composition of claim 4.

17. The method of claim 16, wherein the improved sleep quality is sleep onset latency.

18. The method of claim 16, wherein the improved sleep quality is sleep duration.

19-20. (canceled)

21. A composition comprising:
(i) about 5 mg to about 80 mg of cannabidiol (CBD), about 1 mg to about 30 mg of cannabinol (CBN), and about 1 mg to about 10 mg of Piper nigrum; and
(ii) three or more of the following: about 30 mg to about 180 mg of eicosapentaenoic acid (EPA)/docosahexaenoic acid (DHA), about 80 mg to about 300 mg of Curcuma longa, about 30 mg to about 180 mg of Nigella sativa oil, about 1 mg to about 30 mg of Zanthoxylum spp., about 10 mg to about 80 mg of Citrus reticulata Blanco, about 1 mg to about 15 mg of mixed Tocopherols, about 10 mg to about 80 mg of luteolin, about 0.001 mg to about 1 mg of vitamin D, about 150 mg to about 450 mg of Withania somnifera, about 50 mg to about 220 mg of Corydalis yanhusuo, about 1 mg to about 30 mg of Glycyrrhiza glabra, about 1 mg to about 30 mg of Lavandula angustifolia, about 150 mg to about 400 mg of Scutellaria lateriflora, about 30 mg to about 120 mg of coenzyme Q10, about 50 mg to about 250 mg of Angelica sinensis, about 50 mg to about 200 mg of Ligusticum chuanxiong, about 100 mg to about 300 mg of Salix alba, about 1 mg to about 15 mg of zinc, about 100 mg to about 350 mg of Commiphora mukul, about 0.01 mg to about 1 mg of selenium, about 0.001 mg to about 1 mg of iodine, about 10 mg to about 100 mg of Pueraria lobata, about 100 mg to about 350 mg of Viburnum prunfolium, about 50 mg to about 200 mg of Matricaria recutita, about 1 mg to about 15 mg of Lobelia inflata, about 10 mg to about 80 mg of Melissa officinalis, about 10 mg to about 80 mg of Dioscorea villosa, about 10 mg to about 80 mg of Paeonia lactiflora, and about 1 mg to about 200 mg of Zingiber officinalis.

22-29. (canceled)

30. A method for relieving pain in a subject, comprising administering to the subject the composition of claim 21.

31. The method of claim 30, wherein pain comprises musculoskeletal pain, pain induced by metabolic syndrome, autoimmune pain, chronic pain, diabetic neuropathy pain, polycystic ovary syndrome pain, endometriosis pain, pain induced by cancer, pain induced by inflammation, general pain, pain induced by migraines, pain induced by anxiety, pelvic pain, fibromyalgia pain, pain induced by hypothyroidism, or any combination thereof.

32. A composition comprising:
(i) about 5 mg to about 80 mg of cannabidiol (CBD), about 1 mg to about 30 mg of cannabinol (CBN), about 1 mg to about 30 mg of cannabigerol (CBG), or any combinations thereof;
(ii) one or more of the following: about 1 mg to about 10 mg of Piper nigrum or and about 10 mg to about 150 mg of Citrus reticulata Blanco; and
(iii) two or more of the following: about 10 mg to about 200 mg Lavandula officinalis, about 10 mg to about 250 mg of Polygala tenuifolia, about 100 mg to about 450 mg of Withania somnifera, about 30 mg to about 150 mg of rutaecarpine, about 30 mg to about 150 mg of Passiflora incarnata, about 100 mg to about 400 mg of Ganoderma lucidum, about 50 mg to about 250 mg of Matricaria recutita, about 10 mg to about 150 mg of Pueraria lobata, about 40 mg to 300 mg of Ziziphus spinosa, about 5 mg to about 100 mg of Sceletium tortuosum, about 5 mg to about 50 mg of Crocus sativus, about 100 mg to about 350 mg of Schisandra chinensis, about 80 mg to about 300 mg of dihydromyricetin, about 5 mg to about 80 mg of 5-hydroxytryptophan (5-HTP), about 50 mg to about 300 mg of Poria cocos, about 20 mg to about 200 mg of Panax ginseng, about 100 mg to about 400 mg of Centella asiatica, about 10 mg to about 200 mg of Magnolia officinalis, about 20 mg to about 100 mg of Piper methysticum, and about 30 mg to about 150 mg of paeonol, about 100 to 200 mg of Rosmarinus officinalis, about 50 to 100 mg of Valeriana officinalis, and about 50 to 100 mg of Theacrine.

33-41. (canceled)

42. A method for improving anxiety symptoms in a subject, comprising administering to the subject the composition of claim 32.

43. The method of claim 42, wherein anxiety comprises social anxiety disorder, anxiety due to caffeine excess, generalized anxiety disorder, anxiety in alcoholism, post-traumatic stress disorder, anxiety in the elderly, anxiety induced depression, a specific phobia, obsessive-compulsive disorder, or any combination thereof.

44. (canceled)

45. The composition of claim 4, wherein the composition is formulated for oral delivery.

46-48. (canceled)

49. The composition of claim 4, wherein the composition further comprises about 5 mg to about 175 mg of medium-chain triglyceride (MCT) oil, about 5 mg to about 100 mg of beeswax, about 1 mg to about 30 mg sunflower lecithin, about 0 mg to about 30 mg glycerin, or any combination thereof.

50-57. (canceled)

58. The composition of claim 21, wherein the composition is formulated for oral delivery.

59. The composition of claim 21, wherein the composition further comprises about 5 mg to about 175 mg of medium-chain triglyceride (MCT) oil, about 5 mg to about 100 mg of beeswax, about 1 mg to about 30 mg sunflower lecithin, about 0 mg to about 30 mg glycerin, or any combination thereof.

60. The composition of claim 32, wherein the composition is formulated for oral delivery.

61. The composition of claim 32, wherein the composition further comprises about 5 mg to about 175 mg of medium-chain triglyceride (MCT) oil, about 5 mg to about 100 mg of beeswax, about 1 mg to about 30 mg sunflower lecithin, about 0 mg to about 30 mg glycerin, or any combination thereof.
